(19) United States
(12) Patent Application Publication
(10) Pub. No.: US 2018/0118721 A1
(43) Pub. Date: May 3, 2018

(54) PYRIMIDINE DERIVATIVES

(71) Applicant: Merck Patent GmbH, Darmstadt (DE)

(72) Inventors: Timo HEINRICH, Gross-Umstadt (DE); Ulrich PEHL, Mittelbiberach (DE)

(73) Assignee: Merck Patent GmbH, Darmstadt (DE)

(21) Appl. No.: 15/550,383

(22) PCT Filed: Feb. 12, 2016
(86) PCT No.: PCT/EP2016/000239

§ 371 Date: Aug. 11, 2017

(30) Foreign Application Priority Data
Feb. 13, 2015 (EP) 15000443.0

(51) Int. Cl. C07D 403/04; A61P 35/00; C12Q 1/34; C07D 413/04; C07D 401/04; C07D 491/107; C07D 498/08; C07D 471/04; C07D 405/14; C07D 409/14

(52) U.S. Cl. CPC C07D 403/04; A61P 35/00; C12Q 1/34; C07D 413/04; C07D 401/04; C07D 491/107; C07C 2601/16; C07D 471/04; C07D 405/14; C07D 409/14; C12Q 2521/525; C12Q 2545/114; C07D 498/08

(57) ABSTRACT

Compounds of Formula I or II

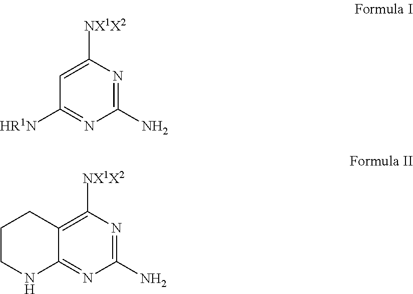

in which R1, X1 and X2 have the meanings indicated in claim 1, are MTH1 inhibitors and can be employed, inter alia, in the treatment of cancer.

### BACKGROUND OF THE INVENTION

The present invention concerns pyrimidine derivatives that are useful as inhibitors of the MTH1 (human mutT homologue 1) protein and their use as medicaments, particularly in the treatment of cancer.

Derivatives of pyrimidine as such have been known as therapeutic or potentially therapeutic agents for a very long time. Fields of medical use cover a large scope, and include medical indications as varied as dermatitis, respiratory diseases, pain, autoimmune diseases, cardiovascular conditions, neurological diseases and overactive bladder. The broad range of medical conditions for which pyrimidine based compounds may be beneficial is associated with various physiological processes where pyrimidine based compounds may come into play.

It is only relatively recently that a certain group of pyrimidine based compounds have been described as providing MTH1 inhibition and eradicating cancer by preventing sanitation of the dNTP (deoxynucleotide triphosphate) pool (Gad, Helleday et al., Nature Vol. 508, 10 Apr. 2014, p. 215, which is incorporated herein in its entirety). Cancers are known as typically having dysfunctional redox regulation resulting in reactive species production that damage both DNA and free dNTPs. The MTH1 protein is described as sanitizing oxidized dNTP pools to prevent incorporation of damaged bases during DNA replication. It was shown that cancer cells require MTH1 activity to avoid incorporation of oxidized dNTPs, which would lead to DNA damage and cell death. Conversely, MTH1 is not essential in normal cells. Hence, the underlying idea is that of targeting MTH1 as a normally non-essential enzyme, which becomes only essential in cancer cells, and thus selectively targeting cancer cells. This is associated with the expectation that dose-limiting side effects could be avoided. Certain featured MTH1 inhibiting compounds were shown to effectively kill cancer cell lines and reduce tumour growth.

The group of pyrimidine compounds identified as providing promising inhibition by the Helleday group share a 2-amino-(N-alkylamino)-6-heteroaryl pyrimidine structure wherein a heteroaryl moiety is connected to the pyrimidine ring via carbon-carbon bonds (WO 2014/084778 A1, which is incorporated herein in its entirety).

Despite constant progress in the development of pharmaceutically active compounds and in the treatment of conditions such as cancer, there remains a need for further and/or improved MTH1 inhibitors. More generally, there remains a need for alternative and/or improved cancer treatments.

Formation of reactive oxygen species (ROS) is also involved in many human pathologic conditions other than cancer, which conditions still require improved, additional or alternative therapies.

### SUMMARY OF THE INVENTION

The present invention provides compounds for use in the treatment of cancer, and in particular a compound of Formula I or Formula II

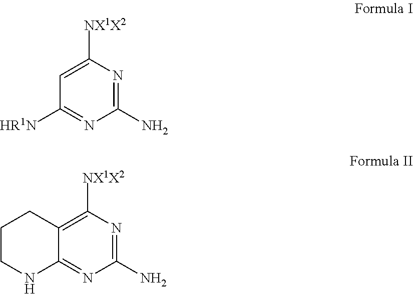

or a pharmaceutically acceptable salt, stereoisomer, tautomer or solvate thereof,
for use in the treatment of cancer wherein
R¹ represents ALK1 optionally substituted by one or more substituents E¹, ALK2 optionally substituted by one or more substituents E³, or ALK3 optionally substituted by one or more substituents E⁴;
   E¹, E³, E⁴ each being independently selected from halogen, hydroxy, oxo (═O), nitro, —CN, —C(O)R^{E1}, —C(O)OR^{E2}, —C(O)NR^{E3}R^{E4}, —OR^{E5}, —OC(O)R^{E6}, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21}, and aryl optionally substituted by one or more substituents E¹¹;
   E¹¹ being independently selected from ALK1 optionally substituted by one or more substituents E²¹, halogen, hydroxy, oxo (═O), nitro, —CN, —C(O)R^{E1}, —C(O)OR^{E2}, —C(O)NR^{E3}R^{E4}, —OR^{E5}, —OC(O)R^{E6}, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21};
   E²¹ being independently selected from halogen, hydroxy, oxo (═O), nitro, —CN, —C(O)R^{E1}, —C(O)OR^{E2}, —C(O)NR^{E3}R^{E4}, —OR^{E5}, —OC(O)R^{E6}, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21}; R^{E1}, R^{E2}, R^{E3}, R^{E4}, R^{E5}, R^{E6}, R^{E7}, R^{E8}, R^{E9}, R^{E10}, R^{E11}, R^{E12}, R^{E13}, R^{E16}, R^{E17}, R^{E18}, R^{E19}, R^{E20} and R^{E21} each being independently selected from H, ALK1, ALK2, ALK3, and aryl, each of which may be optionally substituted by one or more of halogen, hydroxy, oxo (═O), nitro, —CN, and C₁-C₁₂ alkoxy;
   wherein R^{E19} may also be selected from F,
X¹ and X² together with the N to which they are attached form a heterocycle which is selected from:

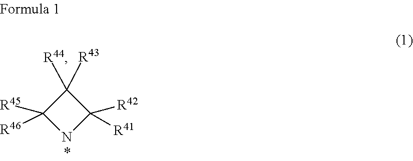

wherein R⁴¹, R⁴², R⁴³, R⁴⁴, R⁴⁵, and R⁴⁶ are independently selected from H, hydroxy, nitro, —CN, halogen, ALK1 optionally substituted by one or more substituents M⁴¹, aryl optionally substituted by one or more substituents M⁴², heterocyclyl optionally substituted by one or more substituents M⁴³, ALK2 optionally substituted by one or more substituents M⁴⁴, ALK3 optionally substituted by one or more substituents M⁴⁵, —C(O)R⁴⁰¹, —C(O)OR⁴⁰², —C(O)NR⁴⁰³R⁴⁰⁴, —OR⁴⁰⁵, —OC(O) R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹ and —S(O)₂NR⁴²⁰R⁴²¹,
or R⁴¹ with R⁴², R⁴³ with R⁴⁴ or R⁴⁵ with R⁴⁶ together form ═O or ═S,
or a combination of R⁴³ and R⁴⁴, R⁴¹ and R⁴², or R⁴⁵ and R⁴⁶ together with the C atom to which they are attached form a 4- to 10-membered carbocyclic or heterocyclic ring system, which ring system is optionally substituted by one or more substituents M⁴⁶,
or a combination of R⁴¹ with R⁴³ or R⁴³ with R⁴⁵ together with the C atoms to which they are attached form a 3- or 4- to 10-membered carbocyclic or heterocyclic ring system, which ring system is optionally substituted by one or more substituents M⁴⁷,
R⁴⁰¹, R⁴⁰², R⁴⁰³, R⁴⁰⁴, R⁴⁰⁵, R⁴⁰⁶, R⁴⁰⁷, R⁴⁰⁸, R⁴⁰⁹, R⁴¹⁰, R⁴¹¹, R⁴¹², R⁴¹³, R⁴¹⁶, R⁴¹⁷, R⁴¹⁸, R⁴¹⁹, R⁴²⁰, R⁴²¹, each being independently selected from H, ALK1 optionally substituted by one or more substituents M⁴⁸, aryl optionally substituted by one or more substituents M⁴⁹,
wherein R⁴¹⁹ in —S(O)₂R⁴¹⁹ may also be F or vinyl,
wherein R⁴⁰¹, R⁴⁰⁵, R⁴⁰⁸ may each independently also be vinyl,
M⁴¹, M⁴⁴, M⁴⁵ and M⁴⁸ each being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and aryl optionally substituted by one or more substituents M^{49a}
M⁴² being independently selected from, halogen, nitro, hydroxy, —C(O)R⁴⁰¹, —C(O)OR⁴⁰², —OR⁴⁰⁵, —OC(O)R⁴⁰⁶, —NR⁴⁰⁷C(O) R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹, ALK1 optionally substituted by one or more substituents M^{48a} and aryl optionally substituted by one or more substituents M^{79a};
M⁴³, M⁴⁹ each being independently selected from, halogen, nitro, hydroxy, —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰9C(O)OR⁴¹⁰, —NR⁴¹1C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and ALK1 optionally substituted by one or more substituents M^{48a};
M⁴⁶ and M⁴⁷ each being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹, ALK1 optionally substituted by one or more substituents M^{48a} and aryl optionally substituted by one or more substituents M^{49a};
M^{48a} being independently selected from halogen, —CN, nitro, hydroxy, oxo (═O), —C(O) R⁴⁰¹, —C(O)OR⁴⁰², —C(O)NR⁴⁰³R⁴⁰⁴, —OR⁴⁰⁵, —OC(O) R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, and —S(O)₂NR⁴²⁰R⁴²¹;
M^{49a} being independently selected from halogen, nitro, hydroxy, oxo (═O), —C(O)R⁴⁰¹, —C(O)OR⁴⁰², —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and ALK1, which is optionally substituted by one or more of halogen, —CN, nitro, hydroxy or C₁₋₁₂ alkoxy;
with the proviso that any N-atom, if present, in addition to the N-atom depicted in above Formula 1 is comprised in the form of a substituent selected from nitro, —CN, —C(O)NR⁴⁰³R⁴⁰⁴, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷ and —S(O)₂NR⁴²⁰R⁴²¹;

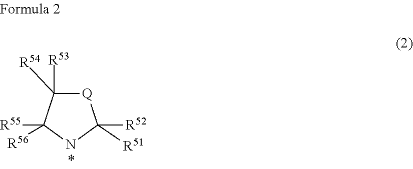

wherein Q is selected from O, S, and CR⁵⁷R⁵⁸,
wherein R⁵¹, R⁵², R⁵³, R⁵⁴, R⁵⁵, R⁵⁶, R⁵⁷, and R⁵⁸ are independently selected from H, hydroxy, nitro, —CN, halogen, ALK1 optionally substituted by one or more substituents M⁵¹, aryl optionally substituted by one or more substituents M⁵², heterocyclyl optionally substituted by one or more substituents M⁵³, ALK2 optionally substituted by one or more substituents M⁵⁴, ALK3 optionally substituted by one or more substituents M⁵⁵, —C(O)R⁵⁰¹, —C(O)OR⁵⁰², —C(O)NR⁵⁰³R⁵⁰⁴, —OR⁵⁰⁵, —OC(O) R⁵⁰⁶, —NR⁵⁰7C(O)R⁵⁰⁸, —NR⁵⁰9C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹
or R⁵¹ with R⁵², R⁵³ with R⁵⁴, R⁵⁵ with R⁵⁶ or R⁵⁷ with R⁵⁸ together form ═O or ═S,
or a combination of R⁵¹ and R⁵², R⁵³ and R⁵⁴, R⁵⁵ and R⁵⁶ or R⁵⁷ and R⁵⁸ together with the C atom to which they are attached form a 4- to 10-membered carbocyclic or heterocyclic ring system, which ring system is optionally substituted by one or more substituents M⁵⁶,
or a combination of R⁵¹ with R⁵⁷, R⁵³ with R⁵⁷, or R⁵³ with R⁵⁵ together with the C atoms to which they are attached form a 3-, 4-, 5-, 6-, 7-, 8-, 9-, or 10-membered carbocyclic or heterocyclic ring system, which ring system is optionally substituted by one or more substituents M⁵⁷,
R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁶, R⁵¹⁷, R⁵¹⁸, R⁵¹⁹, R⁵²⁰, and R⁵²¹ each being independently selected from H, ALK1 optionally substituted by one or more substituents M^{58a} and aryl optionally substituted by one or more substituents M⁵⁹;
wherein R⁵¹⁹ in —S(O)₂R⁴¹⁹ may also be F or vinyl,
wherein R⁵⁰¹, R⁵⁰⁵ and R⁵⁰⁸ may each independently also be vinyl,
M⁵¹, M⁵⁴, M⁵⁵ and M^{58a} each being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O) R⁵⁰⁶, —NR⁵⁰⁷C(O) R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹ and aryl optionally substituted by one or more substituents M^{59a};
M⁵² being independently selected from halogen, nitro, hydroxy, —C(O)R⁵⁰¹, —C(O)OR⁵⁰², —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, ALK1 optionally substituted by one or more substituents M^{58b}, and aryl optionally substituted by one or more substituents M^{59a};
M⁵³ and M⁵⁹ each being independently selected from halogen, nitro, hydroxy, —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, and ALK1 optionally substituted by one or more substituents M^{58b};
M⁵⁶ and M⁵⁷ each being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, ALK1 optionally substituted by one or more substituents M^{58b} and aryl optionally substituted by one or more substituents M^{59a};
M^{58b} being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹;
M^{59a} being independently selected from halogen, nitro, hydroxy, oxo (═O), —C(O)R⁵⁰¹, —C(O)OR⁵⁰², —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹ and ALK1, which is optionally substituted by one or more of halogen, —CN, nitro, hydroxy or C₁₋₁₂ alkoxy;
with the proviso that any N-atom, if present, in addition to the N-atom depicted in above formula 2 is comprised in the form of a substituent selected from nitro, —CN, —C(O)NR⁵⁰³R⁵⁰⁴, —NR⁵⁰⁷C(O) R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, and —S(O)₂NR⁵²⁰R⁵²¹;

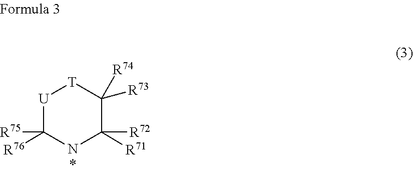

wherein
U is selected from CR⁷⁷R⁷⁸, O and S;
T is selected from CR⁸⁰R⁸¹, O, and S, with the proviso that only one of U and T may be selected from O and S; and
R⁷¹, R⁷², R⁷³, R⁷⁴, R⁷⁵, R⁷⁶, R⁷⁷, R⁷⁸, R⁸⁰ and R⁸¹ are independently selected from H, hydroxy, nitro, —CN, halogen, ALK1 optionally substituted by one or more substituents M⁷¹, aryl optionally substituted by one or more substituents M⁷², heterocyclyl optionally substituted by one or more substituents M⁷³, ALK2 optionally substituted by one or more substituents M⁷⁴, ALK3 optionally substituted by one or more substituents M⁷⁵, —C(O)R⁷⁰¹, —C(O)OR⁷⁰², —C(O)NR⁷⁰³R⁷⁰⁴, —OR⁷⁰⁵, —OC(O) R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, and —S(O)₂NR⁷²⁰R⁷²¹;
or a combination of R⁷¹ and R⁷², R⁷³ and R⁷⁴, R⁷⁵ and R⁷⁶, R⁷⁷ and R⁷⁸, or R⁸⁰ and R⁸¹ together form ═O or ═S,
or a combination of R⁷¹ and R⁷², R⁷³ and R⁷⁴, R⁷⁵ and R⁷⁶, R⁷⁷ and R⁷⁸, or R⁸⁰ and R⁸¹ together with the C atom to which they are attached form a 4- to 10-membered carbocyclic or heterocyclic ring system, which ring system is optionally substituted by one or more substituents M⁷⁶,
or a combination of R⁷² and R⁷⁴, R⁷⁴ and R⁸⁰, R⁸⁰ and R⁷⁸, or R⁷⁸ and R⁷⁶ together with the C atoms to which they are attached form a 3- or 4- to 10-membered carbocyclic or heterocyclic ring system, which ring system is optionally substituted by one or more substituents M⁷⁷,
R⁷⁰¹, R⁷⁰², R⁷⁰³, R⁷⁰⁴, R⁷⁰⁵, R⁷⁰⁶, R⁷⁰⁷, R⁷⁰⁸, R⁷⁰⁹, R⁷¹⁰, R⁷¹¹, R⁷¹², R⁷¹³, R⁷¹⁶, R⁷¹⁷, R⁷¹⁸, R⁷¹⁹, R⁷²⁰ and R⁷²¹ are independently selected from H, ALK1 optionally substituted by one or more substituents M^{78a} and aryl optionally substituted by one or more substituents M⁷⁹;
wherein R⁷¹⁹ in —S(O)₂R⁷¹⁹ may also be F or vinyl,
wherein R⁷⁰¹, R⁷⁰⁵ and R⁷⁰⁸ may each independently also be vinyl,
M⁷¹, M⁷⁴, M⁷⁵ and M^{78a} are each independently selected from hydroxy, oxo (═O), nitro, —CN, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and aryl optionally substituted by one or more substituents M^{79a};
M⁷² each independently selected from hydroxy, nitro, halogen, —C(O)R⁷⁰¹, —C(O)OR⁷⁰², —OR⁷⁰⁵, —OC(O)R⁷⁰⁶, —NR⁷⁰⁷C(O) R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹, ALK1 optionally substituted by one or more substituents M^{78b} and aryl optionally substituted by one or more substituents M^{79a};
M⁷³ and M⁷⁹ each independently selected from hydroxy, nitro, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and ALK1 optionally substituted by one or more substituents M^{78b};
M⁷⁶ and M⁷⁷ each independently selected from hydroxy, oxo (═O), nitro, —CN, halogen, —C(O) R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹, ALK1 optionally substituted by one or more substituents M^{78b} and aryl optionally substituted by one or more substituents M^{79a};
M^{78b} each independently selected from hydroxy, oxo (═O), nitro, —CN, halogen, —C(O) R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R¹⁷, —OS(O)₂R⁷¹⁸, —S(O)ₓR⁷¹⁹, and —S(O)₂NR⁷²⁰R⁷²¹;
M^{79a} each independently selected from hydroxy, oxo (═O), nitro, halogen, —C(O)R⁷⁰¹, —C(O)OR⁷⁰², —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and ALK1, which is optionally substituted by one or more of halogen, —CN, nitro, hydroxy or C₁₋₁₂ alkoxy;
with the proviso that any N-atom, if present, in addition to the N-atom depicted in above Formula 3 is comprised in the form of a substituent selected from nitro, —CN, —C(O)NR⁷⁰³R⁷⁰⁴, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷ and —S(O)₂NR⁷²⁰R⁷²¹
and

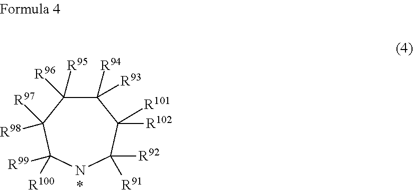

wherein
R⁹¹, R⁹², R⁹³, R⁹⁴, R⁹⁵, R⁹⁶, R⁹⁷, R⁹⁸, R⁹⁹, R¹⁰⁰, R¹⁰¹ and R¹⁰² are independently selected from H, hydroxy, nitro, —CN, halogen, ALK1 optionally substituted by one or more substituents M⁹¹, aryl optionally substituted by one or more substituents M⁹², heterocyclyl optionally substituted by one or more substituents M⁹³, ALK2 optionally substituted by one or more substituents M⁹⁴, ALK3 optionally substituted by one or more substituents M⁹⁵, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹;
or a combination of R⁹¹ and R⁹², R⁹³ and R⁹⁴, R⁹⁵ and R⁹⁶, R⁹⁷ and R⁹⁸, R⁹⁹ and R¹⁰⁰, or R¹⁰¹ and R¹⁰² together forms ═O or ═S,
or R¹⁰¹ and R⁹⁷ together form an oxygen bridge member (—O—),
or a combination of R⁹¹ and R⁹², R⁹³ and R⁹⁴, R⁹⁵ and R⁹⁶, R⁹⁷ and R⁹⁸, or R⁹⁹ and R¹⁰⁰ together with the C atom to which they are attached form a 4- to 10-membered carbocyclic or heterocyclic ring system, which ring system is optionally substituted by one or more substituents M⁹⁶,
or a combination of R⁹¹ and R¹⁰¹, R⁹³ and R¹⁰¹, R⁹³ and R⁹⁵, R⁹⁵ and R⁹⁷, R⁹⁷ and R⁹⁹ together with the C atoms to which they are attached form a 3- or 4- to 10-membered carbocyclic or heterocyclic ring system, which ring system is optionally substituted by one or more substituents M⁹⁷,
R⁹⁰¹, R⁹⁰², R⁹⁰³, R⁹⁰⁴, R⁹⁰⁵, R⁹⁰⁶, R⁹⁰⁷, R⁹⁰⁸, R⁹⁰⁹, R⁹¹⁰, R⁹¹¹, R⁹¹², R⁹¹³, R⁹¹⁶, R⁹¹⁷, R⁹¹⁸, R⁹¹⁹, R⁹²⁰ and R⁹²¹ are each independently selected from H, ALK1 optionally substituted by one or more substituents M^{98a} and aryl optionally substituted by one or more substituents M⁹⁹;
wherein R⁹¹⁹ in —S(O)₂R⁹¹⁹ may also be F or vinyl,
wherein R⁹⁰¹, R⁹⁰⁵ and R⁹⁰⁸ may each independently also be vinyl,
M⁹¹, M⁹⁴, M⁹⁵ and M^{98a} are each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and aryl optionally substituted by one or more substituents M^{99a};
M⁹² is each independently selected from hydroxy, nitro, halogen, —C(O)R⁹⁰¹, —C(O)OR⁹⁰², —OR⁹⁰⁵, —OC(O) R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1 optionally substituted by one or more substituents M^{98b};
M⁹³ and M⁹⁹ are each independently selected from hydroxy, nitro, halogen, —C(O)R⁹⁰¹, —C(O)OR⁹⁰², —C(O)NR⁹⁰³R⁹⁰⁴, —OR⁹⁰⁵, —OC(O) R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1 optionally substituted by one or more substituents M^{98b};
M⁹⁶ and M⁹⁷ are each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹, ALK1 optionally substituted by one or more substituents M^{98b} and aryl optionally substituted by one or more substituents M^{99a};
M^{98b} each independently selected from hydroxy, oxo (═O), nitro, —CN, halogen, —C(O) R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰9C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹, M^{99a} each independently selected from hydroxy, oxo (═O), nitro, halogen, —C(O)R⁹⁰¹, —C(O)OR⁹⁰², —OR⁹⁰⁵, —OC(O)R⁹⁰⁶, —NR⁹⁰⁷C(O) R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1, which is optionally substituted by one or more of halogen, —CN, nitro, hydroxy or C₁₋₁₂ alkoxy,
with the proviso that any N-atom, if present, in addition to the N-atom depicted in above Formula 4 is comprised in the form of a substituent selected from nitro, —CN, —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷ and —S(O)₂NR⁹²⁰R⁹²¹;
and wherein
ALK1 denotes branched or unbranched alkyl having 1, 2, 3, 4, 5, 6, 7, 8, 9, 10, 11 or 12 carbon atoms, cycloalkyl having 3, 4, 5, 6, 7, 8, 9, 10, 11 or 12 carbon atoms, or cycloalkyl substituted alkyl groups having 4, 5, 6, 7, 8, 9, 10, 11 or 12 carbon atoms in total,
ALK2 denotes olefinic groups having 2, 3, 4, 5, 6, 7, 8, 9, 10, 11 or 12 carbon atoms and having one or more double bonds, and includes acyclic branched and unbranched C₂-C₁₂ carbon chains with one or more double bonds, carbocycles having 5, 6, 7, 8, 9 or 10 carbon atoms and one or more double bonds with or without side chains, cycloalkyl substituted acyclic branched and unbranched carbon chains having 5, 6, 7, 8, 9, 10, 11 or 12 carbon atoms in total and cycloalkenyl sustitued alkyl moieties having 6, 7, 8, 9, 10, 11 or 12 carbon atoms in total,
ALK3 denotes branched or unbranched alkynyl having 2, 3, 4, 5, 6, 7, 8, 9, 10, 11 or 12 carbon atoms or cycloalkyl substituted alkynyl having 5, 6, 7, 8, 9, 10, 11 or 12 carbon atoms in total, and
x is 0, 1 or 2.

The star at the N-atom shall emphasize that it is through that N-atom that the various depicted ring systems are attached to the pyrimidine molecule.

As evident from the above Formulas and designation of substituents, the present invention provides pyrimidine compounds which have a saturated N-atom comprising heterocycloalkyl moiety in position 6 of the pyrimidine molecule, which moiety is attached to the pyrimidine molecule via its N-atom. The pyrimidine molecule further comprises an amino group in position 4 as well as position 2 of the pyrimidine molecule, which amino groups, however, are not part of a heterocycloalkyl moiety.

As will be described in more detail further below, compounds of the present invention do not only have shown to have advantageous inhibitory properties for the MTH1 protein, but also have surprisingly good solubilities, which may be associated with improved bioavailability and ease of formulation, and microsomal stability (in vitro clearance) properties. Some of the IC50 values measured are unprecedented (sub-nanomolar range).

In addition, the extent to which compounds of the present invention are able to bind to the target (residence time, calculated on the basis of the KD value) must be regarded as surprising, as will be further shown below.

As used herein, the term “ALK1” encompasses the following:
i) Alkyl groups comprising 1, 2, 3, 4, 5, 6, 7, 8, 9, 10, 11 or 12 carbon atoms. C₁-C₁₂ alkyl groups may be branched, starting from C₃ alkyl, i.e. starting from a chain with 3 carbon atoms, or may be unbranched (straight chain). C₁-C₁₂ alkyl thus encompasses, for example, methyl, ethyl, propyl, iso-propyl, n-propyl, n-butyl, iso-butyl, sec. butyl, tert. butyl, n-propyl, iso-propyl, 1,1-dimethyl-propyl, 1,2-dimethyl-propyl, 2,2-dimethyl-propyl, 1-ethyl-propyl, n-hexyl, iso-hexyl, n-heptyl, iso-heptyl, n-octyl, iso-octyl, n-nonyl, iso-nonyl, neopentyl, n-decyl, iso-decyl, n-undecyl, iso-undecyl-n-dodecyl, iso-dodecyl.
ii) Apart from acyclic branched or unbranched alkyl groups (i), ALK1 also encompasses saturated cycloalkyl groups, i.e. carbocyclic groups having at least 3 and up to 12 carbon atoms, i.e. 3, 4, 5, 6, 7, 8, 9, 10, 11 or 12 carbon atoms. C₃-C₁₂ cycloalkyl groups shall encompass monocyclic, polycyclic and spiro ring system as well as partly cyclic systems. The cycloalkyl group may have alkyl substituents, as long as the total number of carbon atoms does not exceed 12 or may have no alkyl substituents. The carbocyclic C₃- to C₁₂ alkyl groups thus encompass, for example, cyclopentyl, cyclobutyl, cyclopropyl, cyclohexyl, cocyloheptyl, cyclooctyl, spiropentyl, spirohexyl, spiro[4.4]nonyl, spiro[2.6]nonyl, spiro[3.5]nonyl, bicyclohexyl, bicyclo[4.2.0]octyl, bicylo[5.3.0]decyl, tetracyclo[5.2.2.0.0.]undecyl, tricyclo[3.3.1.1]decyl.
iii) The term ALK1 also includes cycloalkyl substituted C₃-C₈ cycloalkyl-C₁-C₈ alkyl groups, i.e. linear or branched alkyl groups substituted with a cycloalkyl group, wherein the total number of 12 carbon atoms is not exceeded. Cycloalkyl substituted alkyl groups comprise 4, 5, 6, 7, 8, 9, 10 or 12 carbonatoms and are connected as a substituent via a carbon atom of the alkyl group. Examples include cyclopropyl-C₁-C₈ alkyl-, such as cyclopropyl-methyl-, cyclobutyl-C₁₋C₈ alkyl, such as cyclobutylethyl, cyclopentyl-C₁-C₇ alkyl, cyclohexyl-C₁-C₆ alkyl, cycloheptyl-C₁-C₅ alkyl and cyclooctyl-C₁-C₄ alkyl groups.

Generally, throughout the description of this invention, among acyclic alkyl groups, C₁-C₆ alkyl groups and in particular those explicitly mentioned above are preferred. Among cyclic structures, monocyclic C₃, C₄, C₅ and C₆ cycloalkyl groups are generally preferred herein.

As used herein, the term ALK2 denotes olefins having 2, 3, 4, 5, 6, 7, 8, 9, 10 or 12 carbon atoms and comprise at least one double bond. Olefins may be acyclic or cyclic. Acyclic and/or cyclic olefins may comprise one double bond only, for instance. Olefinic substituents are preferably connected via a single bond to the moiety to which they are attached. Acyclic olefinic groups may be branched (starting from C₃ alkenyl) or may be unbranched. Acyclic olefins encompass, for instance, vinyl (H₂C═CH—), allyl (prop-2-en-1-yl; H₂C═CH—CH₂—), isopropenyl (H₂C═C(—CH₃)—), but-2-en-1-yl, and but-3-en-1-yl. ALK2 also encompasses carbocycles with 5 to 10 carbon atoms, which comprise at least one double bond, and may optionally have side chains as long as the total carbon number does not exceed 12. ALK2 further includes cycloalkyl substituted acyclic branched or unbranched olefins having 5, 6, 7, 8, 9, 10, 11 or 12 carbon atoms in total, i.e. C₃-C₈ cycloalkyl-C₁-C₈ olefinic groups, such as C₃-C₈ cycloalkyl-C₁-C₈ olefinic groups, such as 1-cyclopropyl-1-propen-2-yl or cycloalkenyl substituted alkyl moieties having from 6 to 12 carbon atoms in total. Acyclic C₂-C₁₂ olefinic groups are generally preferred herein, such as C₂-C₁₂ alkenyl which has one double bond only. In exemplary embodiments, acyclic C₂-C₆ olefinic groups are generally preferred herein, such as C₂-C₆ alkenyl, and in particular those explicitly mentioned above.

As used herein, the abbreviation ALK3 represents a branched or unbranched C₂-C₁₂ alkynyl group, i.e. having 2, 3, 4, 5, 6, 7, 8, 9, 10, 11 or 12 carbon atoms. C₂-C₁₂ alkynyl substituents are connected via a single bond to the moiety to which they are attached. C₂-C₁₂ alkynyl groups may be branched (starting from C₄ alkynyl) or may be unbranched. ALK3 further encompassed cycloalkyl substituted alkynyl having 5, 6, 7, 8, 9, 10, 11 or 12 carbon atoms altogether, such as cyclopropyl-ethinyl, for instance. However, unbranched or branched C₂-C₁₂ alkynyl groups, more particularly, unbranched or branched C₂, C₃, C₄, C₅ orC₆ alkynyl groups are generally preferred herein.

Generally, out of the above moieties ALK1, ALK2, ALK3, typically ALK1 is used.

As used herein, the term “aryl” represents C₆-C₁₄ aromatic groups, including a monocyclic aromatic ring, or a 9 to 14 membered bicyclic or tricyclic ring system wherein at least one ring is aromatic. They may comprise 6, 7, 8, 9, 10, 11, 12, 13 or 14 C atoms. “Aryl” shall comprise only carbocyclic aromatic ring systems, i.e. ring systems wherein the ring members are all carbon atoms, such as phenyl, naphth-1-yl, naphth-2-yl. Phenyl is particularly preferred.

As used herein, the term “heterocyclyl” shall represent both aromatic and non-aromatic mono- or polycyclic ring systems comprising carbon and at is least one heteroatom as ring members, i.e. both hetaryl (heteroaryl) as well as saturated heterocyclyl, i.e. heterocycloalkyl, and unsaturated but non-aromatic heterocyclyl ring systems.

In harmony with the proviso, heterocyclyl groups do not contain nitrogen atoms.

“Hetaryl” or “heteroaryl” as used herein, shall encompass monocyclic aromatic ring systems and bicyclic or tricyclic ring systems wherein at least one ring is aromatic, and which comprise from 1, 2, 3 or 4 heteroatoms selected from oxygen and sulfur. Hetaryl typically include 5- to 10-membered ring systems. Examples of heteroaryl include:
5-membered monocyclic ring systems, such as those based on:

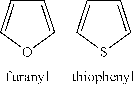

bicyclic ring systems, such as:

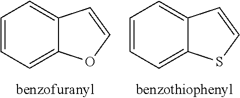

Saturated heterocyclyl groups, i.e. heterocycloalkyl, and unsaturated but non-aromatic heterocyclyl groups are abbreviated “HETALK” rin the following. The encompass non-aromatic monocyclic or polycyclic, e.g. bicyclic, ring systems comprising carbon atoms and at least one heteroatom, for instance, 1 or 2 heteroatoms selected from oxygen (O), and sulfur (S). A HETALK group can be a heterocycloalkyl group and as such be saturated, or alternatively, can be an unsaturated non-aromatic heterocycle have one or more carbon-carbon double bonds or carbon-heteroatoms double bonds in the ring as long as the ring is not rendered aromatic by their presence. For instance, HETALK includes monocyclic ring systems having 3, 4, 5, 6 or 7 ring members, of which 1 or 2 may be heteroatoms, for instance monocyclic ring systems with 1 or 2 heteroatoms. HETALK groups also include bicyclic ring systems, including spiro ring systems with up to 10 ring members and 1 or 2 heteroatoms. Saturated heterocycloalkyl groups are generally preferred in all embodiments herein.

Examples of heterocycloalkyl and unsaturated heterocyclyl groups include, without limitation:
3-membered heterocycloalkyl moieties, such as:

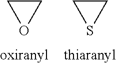

4-membered heterocycloalkyl moieties, such as:

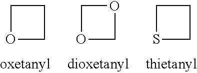

5-membered heterocycloalkyl and unsaturated heterocyclyl moieties, such as:

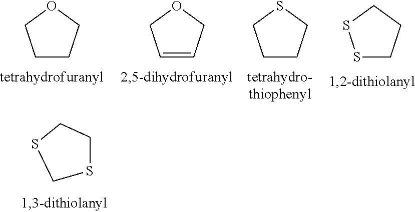

6-membered heterocycloalkyl and unsaturated heterocyclyl moieties, such as:

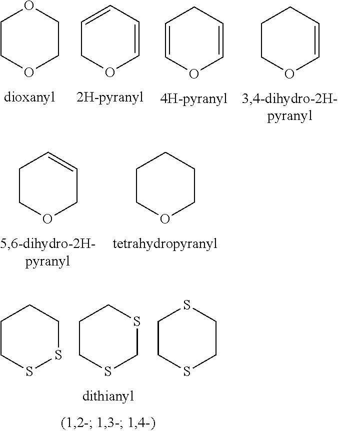

7-membered heterocycloalkyl moieties, such as

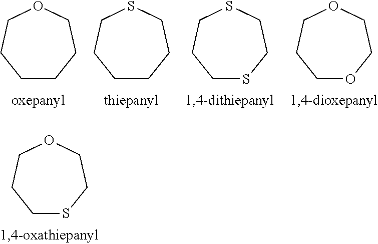

bicyclic heterocycloalkyl moieties, such as: 7-oxabicyclo[2.2.1]heptanyl, 6-oxabicyclo[3.2.1]octanyl

The term “C₁₋₁₂ alkoxy” shall designate a monovalent substituent composed of a C₁-C₁₂ alkyl group (1, 2, 3, 4, 5, 6, 7, 8, 9, 10, 11 or 12 carbon atoms) bonded via a single bond to oxygen: —O—C₁-C₁₂alkyl. The term “alkoxy” shall include halogen substituted alkoxy groups, i.e. C₁₋₁₂ haloalkoxy. Examples of C₁₋₁₂ alkoxy substituents according to the present invention are methoxy, ethoxy, propoxy, butoxy, pentoxy, trifluoromethoxy and trifluoroethoxy.

The term “—CN” designates a nitrile group connected via the carbon atom.

The term “—OH” is used interchangeably with hydroxy or hydroxyl group.

The term halogen, as used herein, typically encompasses fluoro (F), chloro (Cl), bromo (Br) and iodo (I) substituents, with F and Cl generally being preferred. F is most preferred.

The symbol “═O” is used herein to designate an oxo group, “═S” is used herein to designate a thioxo group. Generally, “═O” is preferred over “═S”.

The following substituents can be illustrated by the formulas below:

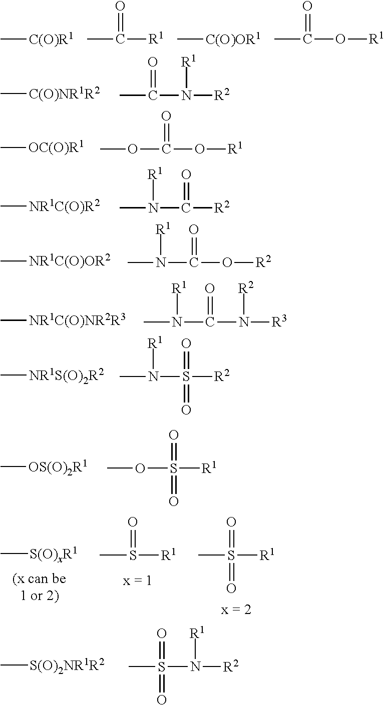

A —S(O)ₓR¹ group wherein x is 0 represents a sulfanyl respectively thiol group.

x can be 0, 1 or 2, and is preferably 2.

“Optionally substituted”, as used herein, means that substitution is optional. The designated moiety can therefore be unsubstituted or substituted. If substituted, any number of hydrogens on the moiety can be replaced with a selection from the indicated possible substituents, provided that the normal valency of the atoms is not exceeded and that a stable compound results.

It is to be understood that, as customary, the various substituents are selected independently of another. In other words, for instance, if more than one substituent of R⁹¹ to R¹⁰² is —C(O)R⁹⁰¹, R⁹⁰¹ can be different for each and any of the substituents. The reference to a particular moiety “R⁹⁰¹” (or any other) is merely a simplified way of referring to a group of substituents and implies an independent selection from the group of moieties encompassed by the variable, irrespective of the particular designation. In other words, there is no interdepence of the substituents.

Typically, the 4, 5, 6, or 7 membered heterocycles formed by NX¹X² in accordance with the present invention are substituted by one, two, three or four substituents, meaning that out of the available R substituents, all but one, two, three or four shall be H. Reference to “at least one” of a certain group of R moieties therefore typically means one, two, three or four of said R moieties are as indicated and the remainder is H. “At least one” R moiety different from H preferably means one, two or three.

In certain embodiments, the present invention provides a compound of Formula I_{[FB1]}

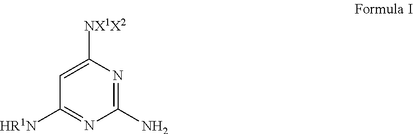

or a pharmaceutically acceptable salt, steroisomer, tautomer or solvate thereof for use in the treatment of cancer,
wherein
R¹ represents ALK1 optionally substituted by one or more substituents E¹, ALK2 optionally substituted by one or more substituents E³, or ALK3 optionally substituted by one or more substituents E⁴;
   E¹, E³, E⁴ each being independently selected from halogen, hydroxy, oxo (═O), nitro, —CN, —C(O)R^{E1}, —C(O)OR^{E2}, —C(O)NR^{E3}R^{E4}, —OR^{E5}, —OC(O)R^{E6}, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21}, and aryl optionally substituted by one or more substituents E¹¹;
   E¹¹ being independently selected from ALK1 optionally substituted by one or more substituents E²¹, halogen, hydroxy, oxo (═O), nitro, —CN, —C(O)R^{E1}, —C(O)OR^{E2}, —C(O)NR^{E3}R^{E4}, —OR^{E5}, —OC(O)R^{E6}, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21};
   E²¹ being independently selected from halogen, hydroxy, oxo (═O), nitro, —CN, —C(O)R^{E1}, —C(O)OR^{E2}, —C(O)NR^{E3}R^{E4}, —OR^{E5}, —OC(O)R^{E6}, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21};
   R^{E1}, R^{E2}, R^{E3}, R^{E4}, R^{E5}, R^{E6}, R^{E7}, R^{E8}, R^{E9}, R^{E10}, R^{E11}, R^{E12}, R^{E13}, R^{E16}, R^{E17}, R^{E18}, R^{E19}, R^{E20} and R^{E21} each being independently selected from H, ALK1, ALK2, ALK3, and aryl, each of which may be optionally substituted by one or more of halogen, hydroxy, oxo (═O), nitro, —CN, and C₁-C₁₂ alkoxy;
      wherein R¹ preferably represents unbranched C₁-C₁₂ alkyl, branched C₁-C₁₂ alkyl, C₃-C₈ cycloalkyl or C₃-C₈ cycloalkyl-C—C₈ alkyl-,
X¹ and X² together with the N to which they are attached form a heterocycle which is selected from:

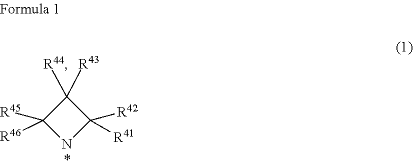

wherein R⁴¹, R⁴², R⁴³, R⁴⁴, R⁴⁵, and R⁴⁶ are independently selected from H, hydroxy, nitro, —CN, halogen, ALK1 optionally substituted by one or more substituents M⁴¹, aryl optionally substituted by one or more substituents M⁴², heterocyclyl optionally substituted by one or more substituents M⁴³, ALK2 optionally substituted by one or more substituents M⁴⁴, ALK3 optionally substituted by one or more substituents M⁴⁵, —C(O)R⁴⁰¹, —C(O)OR⁴⁰², —C(O)NR⁴⁰³R⁴⁰⁴, —OR⁴⁰⁵, —OC(O) R⁴⁰⁶, —NR⁴⁰⁷C(O) R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, and —S(O)₂NR⁴²⁰R⁴²¹
or R⁴¹ with R⁴², R⁴³ with R⁴⁴ or R⁴⁵ with R⁴⁶ together form ═O or ═S,
or a combination of R⁴³ and R⁴⁴, R⁴¹ and R⁴², or R⁴⁵ and R⁴⁶ together with the C atom to which they are attached form a 4- to 10-membered carbocyclic or heterocyclic ring system, which ring system is optionally substituted by one or more substituents M⁴⁶,
or a combination of R⁴¹ with R⁴³ or R⁴³ with R⁴⁵ together with the C atoms to which they are attached form a 4- to 10-membered carbocyclic or heterocyclic ring system, which ring system is optionally substituted by one or more substituents M⁴⁷,
R⁴⁰¹, R⁴⁰², R⁴⁰³, R⁴⁰⁴, R⁴⁰⁵, R⁴⁰⁶, R⁴⁰⁷, R⁴⁰⁸, R⁴⁰⁹, R⁴¹⁰, R⁴¹¹, R⁴¹², R⁴¹³, R⁴¹⁶, R⁴¹⁷, R⁴¹⁸, R⁴¹⁹, R⁴²⁰ and R⁴²¹ each being independently selected from H, ALK1 optionally substituted by one or more substituents M⁴⁸ and aryl optionally substituted by one or more substituents M⁴⁹,
M⁴¹, M⁴⁴, M⁴⁵ and M⁴⁸ each being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and aryl optionally substituted by one or more substituents M^{49a}
M⁴² being independently selected from, halogen, nitro, hydroxy, —C(O)R⁴⁰¹, —C(O)OR⁴⁰², —OR⁴⁰⁵, —OC(O)R⁴⁰⁶, —NR⁴⁰⁷C(O) R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹7, —OS(O)₂R⁴¹⁸, —S(O)ₓR⁴¹⁹, —S(O)₂NR⁴²⁰R⁴²¹ and ALK1 optionally substituted by one or more substituents M^{48a};
M⁴³, M⁴⁹ each being independently selected from, halogen, nitro, hydroxy, —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and ALK1 optionally substituted by one or more substituents M^{48a};
M⁴⁶ and M⁴⁷ each being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹, ALK1 optionally substituted by one or more substituents M^{48a} and aryl optionally substituted by one or more substituents M^{49a};
M^{48a} being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹ 1C(O)NR⁴¹²R⁴¹³, —NR⁴¹⁶S(O)₂R⁴¹⁷, —OS(O)₂R⁴¹⁸, —S(O)ₓR⁴¹⁹, and —S(O)₂NR⁴²⁰R⁴²¹;
M^{49a} being independently selected from halogen, nitro, hydroxy, oxo (═O), —C(O)R⁴⁰¹, —C(O)OR⁴⁰², —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, and —S(O)₂NR⁴²⁰R⁴²¹;
with the proviso that any N-atom, if present, in addition to the N-atom depicted in above Formula 1 is comprised in the form of a substituent selected from nitro, —CN, —C(O)NR⁴⁰³R⁴⁰⁴, —NR⁴⁰⁷C(O) R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, and —S(O)₂NR⁴²⁰R⁴²¹;

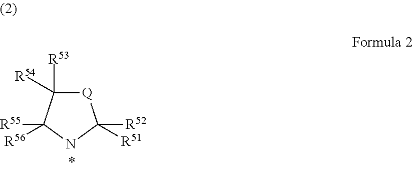

wherein Q is selected from O, S, and CR⁵⁷R⁵⁸,
wherein R⁵¹, R⁵², R⁵³, R⁵⁴, R⁵⁵, R⁵⁶, R⁵⁷, and R⁵⁸ are independently selected from H, hydroxy, nitro, —CN, halogen, ALK1 optionally substituted by one or more substituents M⁵¹, aryl optionally substituted by one or more substituents M⁵², heterocyclyl optionally substituted by one or more substituents M⁵³, ALK2 optionally substituted by one or more substituents M⁵⁴, ALK3 optionally substituted by one or more substituents M⁵⁵, —C(O)R⁵⁰¹, —C(O)OR⁵⁰², —C(O)NR⁵⁰³R⁵⁰⁴, —OR⁵⁰⁵, —OC(O) R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹
or R⁵¹ with R⁵², R⁵³ with R⁵⁴, R⁵⁵ with R⁵⁶ or R⁵⁷ with R⁵⁸ together form ═O or ═S,
or a combination of R⁵¹ and R⁵², R⁵³ and R⁵⁴, R⁵⁵ and R⁵⁶ or R⁵⁷ and R⁵⁸ together with the C atom to which they are attached form a 4- to 10-membered carbocyclic or heterocyclic ring system, which ring system is optionally substituted by one or more substituents M⁵⁶,
or a combination of R⁵¹ with R⁵⁷, R⁵³ with R⁵⁷, or R⁵³ with R⁵⁵ together with the C atoms to which they are attached form a 4- to 10-membered carbocyclic or heterocyclic ring system, which ring system is optionally substituted by one or more substituents M⁵⁷,
R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁶, R⁵¹⁷, R⁵¹⁸, R⁵¹⁹, R⁵²⁰, and R⁵²¹ each being independently selected from H, ALK1 optionally substituted by one or more substituents M^{58a} and aryl optionally substituted by one or more substituents M⁵⁹;
M⁵¹, M⁵⁴, M⁵⁵ and M^{58a} each being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O) R⁵⁰⁶, —NR⁵⁰⁷C(O) R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹ and aryl optionally substituted by one or more substituents M^{59a};
M⁵² being independently selected from halogen, nitro, hydroxy, —C(O)R⁵⁰¹, —C(O)OR⁵⁰², —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, and ALK1 optionally substituted by one or more substituents M^{58b};
M⁵³ and M⁵⁹ each being independently selected from halogen, nitro, hydroxy, —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, and ALK1 optionally substituted by one or more substituents M^{58b};
M⁵⁶ and M⁵⁷ each being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, ALK1 optionally substituted by one or more substituents M^{58b} and aryl optionally substituted by one or more substituents M^{59a};
M^{58b} being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹;
M^{59a} being independently selected from halogen, nitro, hydroxy, oxo (═O), —C(O)R⁵⁰¹, —C(O)OR⁵⁰², —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹;
with the proviso that any N-atom, if present, in addition to the N-atom depicted in above formula 2 is comprised in the form of a substituent selected from nitro, —CN, —C(O)NR⁵⁰³R⁵⁰⁴, —NR⁵⁰⁷C(O) R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, and —S(O)₂NR⁵²⁰R⁵²¹;

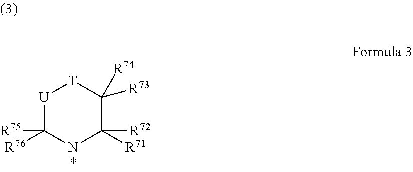

wherein
U is selected from CR⁷⁷R⁷⁸, O and S;
T is selected from CR⁸⁰R⁸¹, O, and S, with the proviso that only one of U and T may be selected from O and S; and
R⁷¹, R⁷², R⁷³, R⁷⁴, R⁷⁵, R⁷⁶, R⁷⁷, R⁷⁸, R⁸⁰ and R⁸¹ are independently selected from H, hydroxy, nitro, —CN, halogen, ALK1 optionally substituted by one or more substituents M⁷¹, aryl optionally substituted by one or more substituents M⁷², heterocyclyl optionally substituted by one or more substituents M⁷³, ALK2 optionally substituted by one or more substituents M⁷⁴, ALK3 optionally substituted by one or more substituents M⁷⁵, —C(O)R⁷⁰¹, —C(O)OR⁷⁰², —C(O)NR⁷⁰³R⁷⁰⁴, —OR⁷⁰⁵, —OC(O) R⁷⁰⁶, —NR⁷⁰7C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, and —S(O)₂NR⁷²⁰R⁷²¹;
or a combination of R⁷¹ and R⁷², R⁷³ and R⁷⁴, R⁷⁵ and R⁷⁶, R⁷⁷ and R⁷⁸, or R⁸⁰ and R⁸¹ together form ═O or ═S,
or a combination of R⁷¹ and R⁷², R⁷³ and R⁷⁴, R⁷⁵ and R⁷⁶, R⁷⁷ and R⁷⁸, or R⁸⁰ and R⁸¹ together with the C atom to which they are attached form a 4- to 10-membered carbocyclic or heterocyclic ring system, which ring system is optionally substituted by one or more substituents M⁷⁶,
or a combination of R⁷² and R⁷⁴, R⁷⁴ and R⁸⁰, R⁸⁰ and R⁷⁸, or R⁷⁸ and R⁷⁶ together with the C atoms to which they are attached form a 4- to 10-membered carbocyclic or heterocyclic ring system, which ring system is optionally substituted by one or more substituents M⁷⁷,
R⁷⁰¹, R⁷⁰², R⁷⁰³, R⁷⁰⁴, R⁷⁰⁵, R⁷⁰⁶, R⁷⁰⁷, R⁷⁰⁸, R⁷⁰⁹, R⁷¹⁰, R⁷¹¹, R⁷¹², R⁷¹³, R⁷¹⁶, R⁷¹⁷, R⁷¹⁸, R⁷¹⁹, R⁷²⁰ and R⁷²¹ are independently selected from H, ALK1 optionally substituted by one or more substituents M^{78a} and aryl optionally substituted by one or more substituents M⁷⁹;
M⁷¹, M⁷⁴, M⁷⁵ and M^{78a} are each independently selected from hydroxy, oxo (═O), nitro, —CN, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and aryl optionally substituted by one or more substituents M^{79a};
M⁷² each independently selected from hydroxy, nitro, halogen, —C(O)R⁷⁰¹, —C(O)OR⁷⁰², —OR⁷⁰⁵, —OC(O)R⁷⁰⁶, —NR⁷⁰⁷C(O) R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹7, —OS(O)₂R⁷¹⁸, —S(O)ₓR⁷¹⁹, —S(O)₂NR⁷²⁰R⁷²¹ and ALK1 optionally substituted by one or more substituents M^{78b};
M⁷³ and M⁷⁹ each independently selected from hydroxy, nitro, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and ALK1 optionally substituted by one or more substituents M^{78b};
M⁷⁶ and M⁷⁷ each independently selected from hydroxy, oxo (═O), nitro, —CN, halogen, —C(O) R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹, ALK1 optionally substituted by one or more substituents M^{78b} and aryl optionally substituted by one or more substituents M^{79a};
M^{78b} each independently selected from hydroxy, oxo (═O), nitro, —CN, halogen, —C(O) R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, and —S(O)₂NR⁷²⁰R⁷²¹;
M^{79a} each independently selected from hydroxy, oxo (═O), nitro, halogen, —C(O)R⁷⁰¹, —C(O)OR⁷⁰², —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, and —S(O)₂NR⁷²⁰R⁷²¹;
with the proviso that any N-atom, if present, in addition to the N-atom depicted in above Formula 3 is comprised in the form of a substituent selected from nitro, —CN, —C(O)NR⁷⁰³R⁷⁰⁴, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷ and —S(O)₂NR⁷²⁰R⁷²¹
and

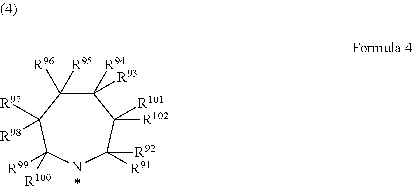

wherein
R⁹¹, R⁹², R⁹³, R⁹⁴, R⁹⁵, R⁹⁶, R⁹⁷, R⁹⁸, R⁹⁹, R¹⁰⁰, R¹⁰¹ and R¹⁰² are independently selected from H, hydroxy, nitro, —CN, halogen, ALK1 optionally substituted by one or more substituents M⁹¹, aryl optionally substituted by one or more substituents M⁹², heterocyclyl optionally substituted by one or more substituents M⁹³, ALK2 optionally substituted by one or more substituents M⁹⁴, ALK3 optionally substituted by one or more substituents M⁹⁵, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹;
or a combination of R⁹¹ and R⁹², R⁹³ and R⁹⁴, R⁹⁵ and R⁹⁶, R⁹⁷ and R⁹⁸, R⁹⁹ and R¹⁰⁰, or R¹⁰¹ and R¹⁰² together forms ═O or ═S,
or R¹⁰¹ and R⁹⁷ together form an oxygen bridge member (—O—),
or a combination of R⁹¹ and R⁹², R⁹³ and R⁹⁴, R⁹⁵ and R⁹⁶, R⁹⁷ and R⁹⁸, or R⁹⁹ and R¹⁰⁰ together with the C atom to which they are attached form a 4- to 10-membered carbocyclic or heterocyclic ring system, which ring system is optionally substituted by one or more substituents M⁹⁶,
or a combination of R⁹¹ and R¹⁰¹, R⁹³ and R¹⁰¹, R⁹³ and R⁹⁵, R⁹⁵ and R⁹⁷, R⁹⁷ and R⁹⁹ together with the C atoms to which they are attached form a 4- to 10-membered carbocyclic or heterocyclic ring system, which ring system is optionally substituted by one or more substituents M⁹⁷,
R⁹⁰¹, R⁹⁰², R⁹⁰³, R⁹⁰⁴, R⁹⁰⁵, R⁹⁰⁶, R⁹⁰⁷, R⁹⁰⁸, R⁹⁰⁹, R⁹¹⁰, R⁹¹¹, R⁹¹², R⁹¹³, R⁹¹⁶, R⁹¹⁷, R⁹¹⁸, R⁹¹⁹, R⁹²⁰ and R⁹²¹ are each independently selected from H, ALK1 optionally substituted by one or more substituents M^{98a} and aryl optionally substituted by one or more substituents M⁹⁹; and
M⁹¹, M⁹⁴, M⁹⁵ and M^{98a} are each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and aryl optionally substituted by one or more substituents M^{99a};
M⁹² is each independently selected from hydroxy, nitro, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O) R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1 optionally substituted by one or more substituents M^{98b};
M⁹³ and M⁹⁹ are each independently selected from hydroxy, nitro, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1 optionally substituted by one or more substituents M^{98b};
M⁹⁶ and M⁹⁷ are each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹, ALK1 optionally substituted by one or more substituents M^{98b} and aryl optionally substituted by one or more substituents M^{99a};
M^{98b} each independently selected from hydroxy, oxo (═O), nitro, —CN, halogen, —C(O) R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹,
M^{99a} each independently selected from hydroxy, oxo (═O), nitro, halogen, —C(O)R⁹⁰¹, —C(O)OR⁹⁰², —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹,
with the proviso that any N-atom, if present, in addition to the N-atom depicted in above Formula 4 is comprised in the form of a substituent selected from nitro, —CN, —C(O)NR⁹⁰³R⁹⁰⁴, —OR⁹⁰⁵, —OC(O)R⁹⁰⁶, —NR⁹⁰7C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷ and —S(O)₂NR⁹²⁰R⁹²¹;
and wherein
ALK1 denotes branched or unbranched alkyl having from 1 to 12 carbon atoms, cycloalkyl having from 3 to 12 carbon atoms, or cycloalkyl substituted alkyl groups having from 4 to 12 carbon atoms in total,
ALK2 denotes olefinic groups having from 2 to 12 carbon atoms and having one or more double bonds, and includes acyclic branched and unbranched C₂-C₁₂ carbon chains with one or more double bonds, carbocycles having from 5 to 10 carbon atoms and one or more double bonds with or without side chains, cycloalkyl substituted acyclic branched and unbranched carbon chains having from 5 to 12 carbon atoms in total and cycloalkenyl sustitued alkyl moieties having from 6 to 12 carbon atoms in total,
ALK3 denotes branched or unbranched alkynyl having from 2 to 12 carbon atoms or cycloalkyl substituted alkynyl having from 5 to 12 carbon atoms in total,
x is 1 or 2.

For a compound of Formula I with each and any group NX1X2, R¹ preferably represents ALK1, in particular unbranched C₁-C₁₂ alkyl, branched C₁-C₁₂ alkyl, C₃-C₈ cycloalkyl or C₃-C₈ cycloalkyl-C₁-C₈ alkyl-, or ALK1 substituted by aryl, preferably phenyl, which aryl may optionally be substituted as set out above, preferably by one or more substituents independently selected from branched or unbranched C₁-C₁₂ alkyl, C₁-C₁₂ alkoxy, halogen, hydroxy, nitro, and —CN.

For a compound of Formula I with any group NX1X2, R¹ more preferably represents unbranched C₁-C₁₂ alkyl, branched C₁-C₁₂ alkyl, C₃-C₈ cycloalkyl or C₃-C₈ cycloalkyl-C₁-C₈ alkyl-.

In certain preferred embodiments of compounds according to Formula I, NHR1 represents methylamino, as illustrated by the following Formula III

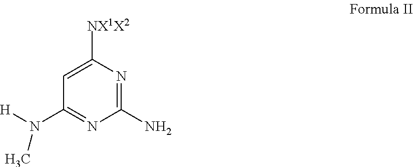

In certain other embodiments of compounds according to Formula I, NHR1 represents cyclopropylamino, as illustrated by Formula IV below:

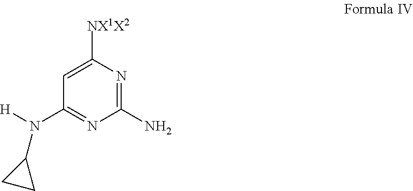

In further exemplary embodiments R¹ may be ethyl, isopropyl, cyclopropyl-methyl-, cyclopentyl-methyl-, or C1-C12 alkoxy-phenyl-ethyl-, such as (2-methoxy-phenyl)-ethyl-.

In certain embodiments, in each and any compound according to one of Formulas I, II, III, and IV, X¹ and X² together with the N to which they are attached may form a heterocycle according to Formula 1

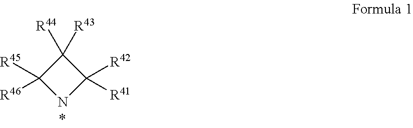

wherein R⁴¹, R⁴², R⁴³, R⁴⁴, R⁴⁵, and R⁴⁶ are independently selected from H, hydroxy, nitro, —CN, halogen, ALK1 optionally substituted by one or more substituents M⁴¹, aryl optionally substituted by one or more substituents M⁴², heterocyclyl optionally substituted by one or more substituents M⁴³, ALK2 optionally substituted by one or more substituents M⁴⁴, ALK3 optionally substituted by one or more substituents M⁴⁵, —C(O)R⁴⁰¹, —C(O)OR⁴⁰², —C(O)NR⁴⁰³R⁴⁰⁴, —OR⁴⁰⁵, —OC(O) R⁴⁰⁶, —NR⁴⁰⁷C(O) R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹ and —S(O)₂NR⁴²⁰R⁴²¹
or R⁴¹ with R⁴², R⁴³ with R⁴⁴ or R⁴⁵ with R⁴⁶ together form ═O or ═S,
or a combination of R⁴³ and R⁴⁴, R⁴¹ and R⁴², or R⁴⁵ and R⁴⁶ together with the C atom to which they are attached form a 4- to 10-membered carbocyclic or heterocyclic ring system, which ring system is optionally substituted by one or more substituents M⁴⁶,
or a combination of R⁴¹ with R⁴³ or R⁴³ with R⁴⁵ together with the C atoms to which they are attached form a 3- or 4- to 10-membered carbocyclic or heterocyclic ring system, which ring system is optionally substituted by one or more substituents M⁴⁷,
R⁴⁰¹, R⁴⁰², R⁴⁰³, R⁴⁰⁴, R⁴⁰⁵, R⁴⁰⁶, R⁴⁰⁷, R⁴⁰⁸, R⁴⁰⁹, R⁴¹⁰, R⁴¹¹, R⁴¹², R⁴¹³, R⁴¹⁶, R⁴¹⁷, R⁴¹⁸, R⁴¹⁹, R⁴²⁰, R⁴²¹, each being independently selected from H, ALK1 optionally substituted by one or more substituents M⁴⁸, aryl optionally substituted by one or more substituents M⁴⁹,
wherein R⁴¹⁹ in —S(O)₂R⁴¹⁹ may also be F or vinyl,
wherein R⁴⁰¹, R⁴⁰⁵, R⁴⁰⁸ may each independently also be vinyl,
M⁴¹, M⁴⁴, M⁴⁵ and M⁴⁸ each being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O) R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and aryl optionally substituted by one or more substituents M^{49a},
M⁴² being independently selected from, halogen, nitro, hydroxy, —C(O)R⁴⁰¹, —C(O)OR⁴⁰², —OR⁴⁰⁵, —OC(O)R⁴⁰⁶, —NR⁴⁰⁷C(O) R⁴⁰⁸, —NR⁴⁰9C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹, ALK1 optionally substituted by one or more substituents M^{48a} and aryl optionally substituted by one or more substituents M^{79a};
M⁴³, M⁴⁹ each being independently selected from, halogen, nitro, hydroxy, —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and ALK1 optionally substituted by one or more substituents M^{48a};
M⁴⁶ and M⁴⁷ each being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O) R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹, ALK1 optionally substituted by one or more substituents M^{48a} and aryl optionally substituted by one or more substituents M^{49a};
M^{48a} being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O) R⁴⁰⁶, —NR⁴⁰⁷C(O)R⁴⁰⁸, —NR⁴⁰9C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, and —S(O)₂NR⁴²⁰R⁴²¹;
M^{49a} being independently selected from halogen, nitro, hydroxy, oxo (═O), —C(O)R⁴⁰¹, —C(O)OR⁴⁰², —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and ALK1, which is optionally substituted by one or more of halogen, —CN, nitro, hydroxy or C₁₋₁₂ alkoxy;
wherein x is 0, 1 or 2, preferably 2,
with the proviso that any N-atom, if present, in addition to the N-atom depicted in above Formula 1 is comprised in the form of a substituent selected from nitro, —CN, —C(O)NR⁴⁰³R⁴⁰⁴, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷ and —S(O)₂NR⁴²⁰R⁴²¹;

For instance, at least 2, or at least 3, or at least 4, or at least 5 of R⁴¹, R⁴², R⁴³, R⁴⁴, R⁴⁵, and R⁴⁶ may be hydrogen. Preferably, at least 4, or at least 5 of R⁴¹, R⁴², R⁴³, R⁴⁴, R⁴⁵, and R⁴⁶ are hydrogen.

For instance, the azetidinyl moiety according to above Formula 1 may be mono- or disubstituted, i.e. 4 to 5 of R⁴¹, R⁴², R⁴³, R⁴⁴, R⁴⁵, and R⁴⁶ are H. and, e.g. one or two of R⁴¹, R⁴², R⁴³, R⁴⁴, R⁴⁵, and R⁴⁶ are different from H, and are preferably independently selected from fluoro, chloro, hydroxyl, C₁-C₁₂ alkoxy, phenyl, substituted phenyl, halogen-substituted phenyl, benzyl, substituted benzyl, halogen-substituted benzyl, —C(O)—NH—(CH₂)₂—(C₆H₄)—S(O)₂F, —C(O)—NH—(CH₂)₂—(C₆H₄)—NH—C(O)—CH═CH₂; or two of R⁴¹, R⁴², R⁴³, R⁴⁴, R⁴⁵, and R⁴⁶ together form an oxo group or a spiro group. As evident from the examples below and in Table 1, one of R⁴³ and R⁴⁴ may be H—C(O)—NH—(CH₂)₂—(C₆H₄)—S(O)₂F or —C(O)—NH—(CH₂)₂—(C₆H₄)—NH—C(O)—CH═CH₂ whereas the other one of R⁴³ and R⁴⁴ as well as R⁴¹, R⁴², R⁴⁵, and R⁴⁶ are then preferably H.

If a combination of R⁴³ and R⁴⁴, R⁴¹ and R⁴², or R⁴⁵ and R⁴⁶ together with the C atom to which they are attached form a 4-, 5-, 6-, 7-, 8-, 9- or 10-membered carbocyclic or heterocyclic ring system, which ring system is optionally substituted by one or more substituents M⁴⁶, said ring system may be saturated. If the ring system thus formed is heterocyclic, it preferably contains O, typically one oxygen atom only. In harmony with the proviso, said ring system may not comprise a N ring member. The 4- to 10-membered carbocyclic ring system may be a 4-, 5-, 6- or 7-membered monocyclic ring system, for instance, or may be a 7-, 8-, 9- or 10-membered bicyclic ring system. The ring-system may comprise a saturated carbocyclic or heterocyclic 3-, 4-, 5- or 6-membered ring fused to a benzene ring, for instance. Examples of 3- or 4- to 10-membered carbocyclic or heterocyclic ring systems thus formed include some of the 3- or 4- to 7-membered monocyclic heterocycloalky moieties and bicyclic heterocycloalkyl moieties set out above, which may optionally be annealed to a benzene ring, as long as the ring system thus formed does not comprise more than 10 ring atoms.

If a combination of R⁴¹ with R⁴³ or R⁴³ with R⁴⁵ together with the C atoms to which they are attached form a 4-, 5-, 6-, 7-, 8-, 9- or 10-membered carbocyclic or heterocyclic ring system, which ring system is optionally substituted by one or more substituents M⁴⁷, said ring system may be saturated, for instance. If the ring system thus formed is heterocyclic, it preferably contains O, preferably only one oxygen atom. In harmony with the proviso, said ring system may not comprise a N ring member. In the alternative, the ring system may include a benzene ring annealed to a carbocycle or heterocycle, which carbocyle or heterocycle include the R⁴¹ with R⁴³ or R⁴³ with R⁴⁵ together with the C atoms to which they are attached. Examples of 3- to 10-membered carbocyclic or heterocyclic ring systems thus formed include the 3- or 4 to 7-membered monocyclic heterocycloalky moieties and bicyclic heterocycloalkyl moieties set out above, which may optionally be annealed to a benzene ring, as long as the ring system thus formed does not comprise more than 10 ring atoms. In a further alternative embodiment, the ring system formed by combination of R⁴¹ with R⁴³ or R⁴³ with R⁴⁵ together with the C atoms to which they are attached may be or comprise a benzene ring.

As expressed by the proviso, R⁴¹, R⁴², R⁴³, R⁴⁴, R⁴⁵, and R⁴⁶ including any substituents, if present, are selected such that the azetidinyl moiety according to Formula 1 does not comprise an amino group or any further N ring members (in any spiro or annealed group). Any N-atom, if present, in addition to the N-atom depicted in above Formula 1 is comprised in the form of a substituent selected from nitro, —CN, —C(O)NR⁴⁰³R⁴⁰⁴, —NR⁴⁰⁷C(O)R⁴⁰⁸, —NR⁴⁰⁹C(O)OR⁴¹⁰, —NR⁴¹¹C(O)NR⁴¹²R⁴¹³, and —NR⁴¹⁶S(O)₂R⁴¹⁷. Of course, these substituents may be used if and where the respective definitions of R and M foresee it.

In preferred embodiments, at least one of R⁴¹, R⁴², R⁴³, R⁴⁴, R⁴⁵, and R⁴⁶ is selected from —O—CH₃, —O—CH₂—CH₃, —O—(C₁₋₆ alkyl), —O-ALK1, —CH₂—O—CH₃, —(CH₂)₂₋₄—O—(CH₂)₀₋₄CH₃, —CH₂—S—CH₃, —OH, —CH₂—OH, —(CH₂)₂₋₄—OH, —CF₃, —CH₂—Br, —(CH₂)₂₋₄—Br, —F, —Cl, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—S(O)₂F, —C(O)—NH—(CH₂)₂—(C₆H₄)—NH—C(O)—O—C(CH₃)₃, —C(O)—NH—(CH₂)₂—(C₆H₄)—NH—C(O)—CH═CH₂, —(C₆H₄)—NH—C(O)—CH═CH₂, (C₆H₄)—C(O)—CH═CH₂, —(C₆H₄)—CH═O, —(C₆H₄)—S(O)₂—CH═CH₂, —(C₆H₄)—F, —(C₆H₄)—S(O)₂F, —O—(CH₂)₂—(C₆H₅); —C(O)—O—(C₆F₅), —CH₂—C(O)—O—(C₆F₅), —CH═O, and allyl. Vice versa, the remainder of the positions would be constituted by H.

In certain other preferred embodiments, R⁴¹, R⁴², R⁴³, R⁴⁴, R⁴⁵, and R⁴⁶ including any substituents, if present, are selected such that the N atom depicted in above Formula 1 is the only N atom comprised by Formula 1.

For instance, in the compounds according to each of Formula I or II, as well as III and IV, X¹ and X² together with the N to which they are attached may form an azetidinyl structure according to the following Formulas 1a to 1o:

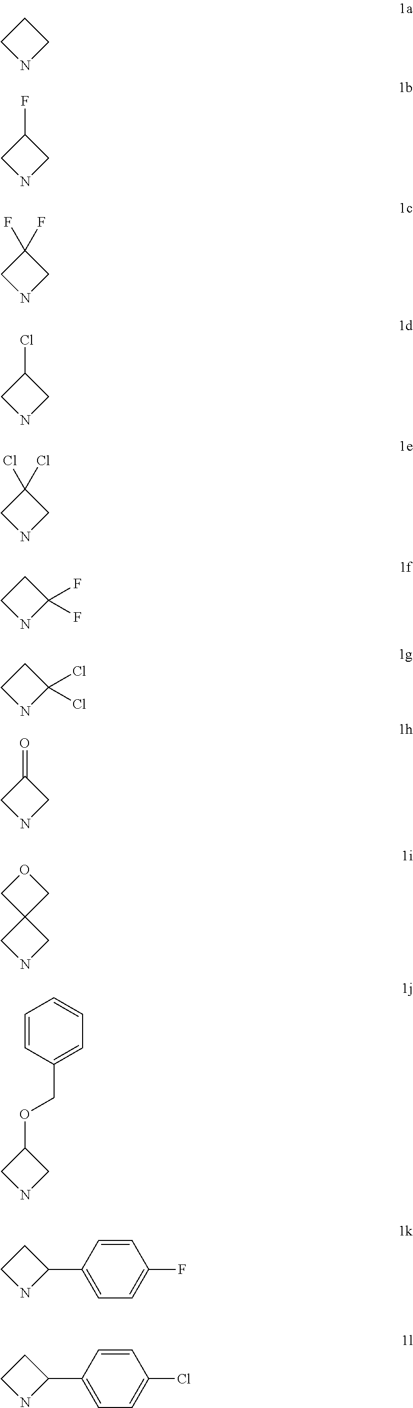

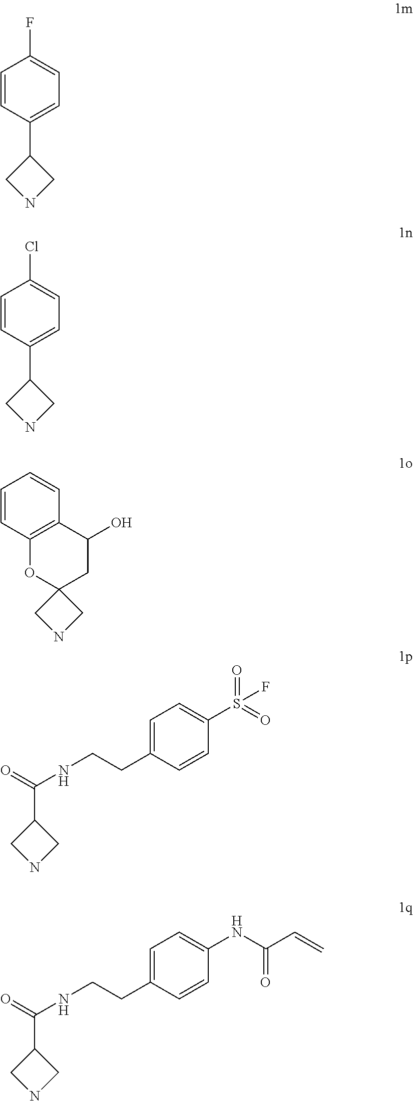

Particularly preferred are the azetidinyl moieties according to above Formulas 1a, 1c, 1h, 1i, 1k, 1p and 1q.

The present invention thus encompasses, amongst others, the following exemplary embodiment of compounds according to Formula I, respectively groups of compounds:

| R¹ | NX¹X² |
|---|---|
| Opt. subst. C₁-C₆ alkyl | Formula 1 |
| Opt. subst. C₁-C₆ alkyl | 1a-1q |
| Opt. subst. C₁-C₆ alkyl | Fqrmula 1 |
| Opt. subst. C₁-C₆ alkyl | 1a-1q |
| Opt. subst. C₁-C₆ alkyl | Fqrmula 1 |
| Opt. subst. C₁-C₆ alkyl | 1a-1q |
| Opt. subst. C₁-C₆ alkyl | Fqrmula 1 |
| Opt. subst. C₁-C₆ alkyl | 1a-1q |
| Opt. subst. C₁-C₆ alkyl | Fqrmula 1 |
| Opt. subst. C₁-C₆ alkyl | 1a-1q |
| Opt. subst. C₃ or C₄ cycloalkyl | Fqrmula 1 |
| Opt. subst. C₃ or C₄ cycloalkyl | 1a-1q |
| Opt. subst. C₃ or C₄ cycloalkyl | Fqrmula 1 |
| Opt. subst. C₃ or C₄ cycloalkyl | 1a-1q |
| Opt. subst. C₃ or C₄ cycloalkyl | Fqrmula 1 |
| Opt. subst. C₃ or C₄ cycloalkyl | 1a-1q |
| Opt. subst. C₃ or C₄ cycloalkyl | Fqrmula 1 |
| Opt. subst. C₃ or C₄ cycloalkyl | 1a-1q |
| Opt. subst. C₃ or C₄ cycloalkyl | Fqrmula 1 |
| Opt. subst. C₃ or C₄ cycloalkyl | 1a-1q |
| —C₂H₅ | Fqrmula 1 |
| —C₂H₅ | 1a-1q |
| isopropyl | Fqrmula 1 |
| isopropyl | 1a-1q |
| cyclopropyl-methyl- | Fqrmula 1 |
| cyclopropyl-methyl- | 1a-1q |
| cyclopentyl-methyl- | Fqrmula 1 |
| cyclopentyl-methyl- | 1a-1q |
| ALK 1, substituted with unsubstituted or substituted phenyl | Fqrmula 1 |
| ALK 1, substituted with unsubstituted or substituted phenyl | 1a-1q |
| —C₂H_{4—}(unsubstituted or substituted phenyl) | Fqrmula 1 |
| —C₂H_{4—}(unsubstituted or substituted phenyl) | 1a-1q |

In alternative embodiments, in each and any compound according to one of above Formulas I, II, III, and IV, X¹ and X² together with the N to which they are attached may form a heterocycle according to Formula 2:

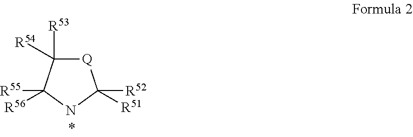

wherein Q is selected from O, S, and CR⁵⁷R⁵⁸,
wherein R⁵¹, R⁵², R⁵³, R⁵⁴, R⁵⁵, R⁵⁶, R⁵⁷, and R⁵⁸ are independently selected from H, hydroxy, nitro, —CN, halogen, ALK1 optionally substituted by one or more substituents M⁵¹, aryl optionally substituted by one or more substituents M⁵², heterocyclyl optionally substituted by one or more substituents M⁵³, ALK2 optionally substituted by one or more substituents M⁵⁴, ALK3 optionally substituted by one or more substituents M⁵⁵, —C(O)R⁵⁰¹, —C(O)OR⁵⁰², —C(O)NR⁵⁰³R⁵⁰⁴, —OR⁵⁰⁵, —OC(O) R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹
or R⁵¹ with R⁵², R⁵³ with R⁵⁴, R⁵⁵ with R⁵⁶ or R⁵⁷ with R⁵⁸ together form ═O or ═S,
or a combination of R⁵¹ and R⁵², R⁵³ and R⁵⁴, R⁵⁵ and R⁵⁶ or R⁵⁷ and R⁵⁸ together with the C atom to which they are attached form a 4- to 10-membered carbocyclic or heterocyclic ring system, which ring system is optionally substituted by one or more substituents M⁵⁶,
or a combination of R⁵¹ with R⁵⁷, R⁵³ with R⁵⁷, or R⁵³ with R⁵⁵ together with the C atoms to which they are attached form a 3-, 4-, 5-, 6-, 7-, 8-, 9-, or 10-membered carbocyclic or heterocyclic ring system, which ring system is optionally substituted by one or more substituents M⁵⁷,
R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁶, R⁵¹⁷, R⁵¹⁸, R⁵¹⁹, R⁵²⁰, and R⁵²¹ each being independently selected from H, ALK1 optionally substituted by one or more substituents M^{58a} and aryl optionally substituted by one or more substituents M⁵⁹;
wherein R⁵¹⁹ in —S(O)₂R⁴¹⁹ may also be F or vinyl,
wherein R⁵⁰¹, R⁵⁰⁵ and R⁵⁰⁸ may each independently also be vinyl,
M⁵¹, M⁵⁴, M⁵⁵ and M^{58a} each being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O) R⁵⁰⁶, —NR⁵⁰⁷C(O) R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹ and aryl optionally substituted by one or more substituents M⁵⁹a;
M⁵² being independently selected from halogen, nitro, hydroxy, —C(O)R⁵¹, —C(O)OR⁵⁰², —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, ALK1 optionally substituted by one or more substituents M^{58b}, and aryl optionally substituted by one or more substituents M^{59a};
M⁵³ and M⁵⁹ each being independently selected from halogen, nitro, hydroxy, —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰7C(O)R⁵⁰⁸, —NR⁵⁰9C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, and ALK1 optionally substituted by one or more substituents M^{58b};
M⁵⁶ and M⁵⁷ each being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, ALK1 optionally substituted by one or more substituents M^{58b} and aryl optionally substituted by one or more substituents M^{59a};
M^{58b} being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹;
M^{59a} being independently selected from halogen, nitro, hydroxy, oxo (═O), —C(O)R⁵⁰¹, —C(O)OR⁵⁰², —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹ and ALK1, which is optionally substituted by one or more of halogen, —CN, nitro, hydroxy or C₁₋₁₂ alkoxy;
with the proviso that any N-atom, if present, in addition to the N-atom depicted in above formula 2 is comprised in the form of a substituent selected from nitro, —CN, —C(O)NR⁵⁰³R⁵⁰⁴, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, and —S(O)₂NR⁵²⁰R⁵²¹;

As generally defined herein, x may be 0, 1 or 2 and is preferably 2.

For instance, at least 2, or at least 3, or at least 4, or at least 5 of R⁵¹, R⁵², R⁵³, R⁵⁴, R⁵⁵, R⁵⁶ may be H.

In particularly preferred embodiments, Q is CR⁵⁷R⁵⁸ (pyrrolidine moiety).

In certain embodiments, Q is CR⁵⁷R⁵⁸ and R⁵¹, R⁵², R⁵³, R⁵⁴, R⁵⁵, R⁵⁶, R⁵⁷, and R⁵⁸ are independently selected from H, hydroxy, nitro, —CN, halogen, ALK1 optionally substituted by one or more substituents M⁵¹, aryl optionally substituted by one or more substituents M⁵², heterocyclyl optionally substituted by one or more substituents M⁵³, ALK2 optionally substituted by one or more substituents M⁵⁴, ALK3 optionally substituted by one or more substituents M⁵⁵, —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹,

or R⁵¹ with R⁵², R⁵³ with R⁵⁴, R⁵⁵ with R⁵⁶ or R⁵⁷ with R⁵⁸ together form ═O or ═S,
or a combination of R⁵¹ and R⁵², R⁵³ and R⁵⁴, R⁵⁵ and R⁵⁶ or R⁵⁷ and R⁵⁸ together with the C atom to which they are attached form a 3- or 4- to 10-membered carbocyclic or heterocyclic ring system, which ring system is optionally substituted by one or more substituents M⁵⁶,
or a combination of R⁵¹ with R⁵⁷, R⁵³ with R⁵⁷, or R⁵³ with R⁵⁵ together with the C atoms to which they are attached form a 3- or 4- to 10-membered carbocyclic or heterocyclic ring system, which ring system is optionally substituted by one or more substituents M⁵⁷,
more preferably:
R⁵¹, R⁵², R⁵³, R⁵⁴, R⁵⁵, R⁵⁶, R⁵⁷, and R⁵⁸ are independently selected from H, hydroxy, halogen, C₁-C₁₂ alkyl, more preferably C₁-C₆ alkyl, optionally substituted by one or more substituents M⁵¹, aryl optionally substituted by one or more substituents M⁵², —C(O)R⁵⁰¹, —C(O)OR⁵⁰², —C(O)NR⁵⁰³R⁵⁰⁴, —OR⁵⁰⁵, —OC(O) R⁵⁰⁶, —NR⁵⁰⁷C(O) R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹.
or R⁵¹ with R⁵², R⁵³ with R⁵⁴, R⁵⁵ with R⁵⁶ or R⁵⁷ with R⁵⁸ together form ═O or ═S,
or a combination of R⁵¹ and R⁵², R⁵³ and R⁵⁴, R⁵⁵ and R⁵⁶ or R⁵⁷ and R⁵⁸ together with the C atom to which they are attached form a 4- to 7-membered carbocyclic ring, which ring is optionally substituted by one or more substituents M⁵⁶, and
R⁵⁰¹, R⁵⁰², R⁵⁰³, R⁵⁰³, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, and R⁵²¹ are each independently selected from H, optionally substituted C₁-C₁₂ alkyl, more preferably optionally substituted C₁-C₆ alkyl and optionally substituted aryl,
wherein optionally R⁵¹⁹ in —S(O)₂R⁴¹⁹ may also be F or vinyl, wherein optionally R⁵⁰¹, R⁵⁰⁵ and R⁵⁰⁸ may each independently also be vinyl.

In certain preferred embodiments of compounds of any of Formulas I, II, III or IV, Q is selected from O, S, and CR⁵⁷R⁵⁸, preferably CR⁵⁷R⁵⁸, and R⁵¹, R⁵², R⁵³, R⁵⁴, R⁵⁵, R⁵⁶, R⁵⁷, and R⁵⁸ (including substituents, if any) are selected such that the N atom depicted in above Formula 2 is the only N atom comprised by Formula 2.

In certain embodiments of compounds of any of Formulas, II, II or IV, is most preferably CR⁵⁷R⁵⁸, and at least one of R⁵¹, R⁵², R⁵³: R⁵⁵, R⁵⁶, R⁵⁷, R and R⁵⁸ is selected from hydroxy, hydroxy-substituted C₁-C₆ alkyl, such as hydroxymethyl, hydroxyethyl, hydroxypropy, halogen, such a fluoro, chloro, selected from H, methy, ethyl, propyl and R⁵⁰⁸ independently selected from methy, ethyl, propy, —C(O)NR⁵⁰³R⁵⁰⁴ With R⁵⁰³ selected from H, methyl, ethyl, propyl, and R⁵⁰⁴ independently selected from H, methyl, ethyl, propyl._{[FB2]}

In particularly preferred embodiments of compounds of any of Formulas I, II, III or IV, Q is most preferably CR⁵⁷R⁵⁸, and at least one of R⁵¹, R⁵², R⁵³, R⁵⁴, R⁵⁵, R⁵⁶, R⁵⁷, and R⁵⁸ is selected from unsubstituted phenyl or phenyl substituted with one or more of halogen, preferably F and/or Cl, -hydroxy, C₁-C₆ alkoxy, methoxy, C₁-C₆ haloalkoxy, —S(O)₂F, —S(O)₂CH═CH₂, —NH—C(O)—CH═CH₂, —C(O)—CH═CH₂ and —CH(═O); unsubstituted benzyl or benzyl_{[FB3]} substituted with one or more of halogen, preferably F and/or Cl, -hydroxy, C₁-C₆ alkoxy, methoxy, C₁-C₆ haloalkoxy, —S(O)₂F, —S(O)₂CH═CH₂, —NH—C(O)—CH═CH₂, —C(O)—CH═CH₂, and —CH(═O); or unsubstituted phenylethyl or phenethyl substituted with one or more of halogen, preferably F and/or Cl, -hydroxy, methoxy, C₁-C₆ alkoxy, C₁-C₆ haloalkoxy, —S(O)₂F, —S(O)₂CH═CH₂, —NH—C(O)—CH═CH₂, —C(O)—CH═CH₂, and —CH(═O). In those embodiments, wherein at least one of R⁵¹, R⁵², R⁵³, R⁵⁴, R⁵⁵, R⁵⁶, R⁵⁷, and R⁵⁸ is selected from optionally substituted phenyl, benzyl or phenethyl, a least another one of R⁵¹, R⁵², R⁵³, R⁵⁴, R⁵⁵, R⁵⁶, R⁵⁷, and R⁵⁸ is preferably selected from hydroxy, C₁₋₆ alkoxy, halogen, in particular F or Cl, or oxo.

If a combination of R⁵¹ and R⁵², R⁵³ and R⁵⁴, R⁵⁵ and R⁵⁶ or R⁵⁷ and R⁵⁸ together with the C atom to which they are attached form a 4- to 10-membered carbocyclic or heterocyclic ring system optionally substituted by one or more substituents M⁵⁶ or a combination of R⁵¹ with R⁵⁷, R⁵³ with R⁵⁷, or R⁵³ with R⁵⁵ together with the C atoms to which they are attached form a 3- or 4- to 10-membered carbocyclic or heterocyclic ring system optionally substituted by one or more substituents M⁵⁷, then the 3- or 4- to 10-membered carbocyclic or heterocyclic ring system may be saturated. If the ring system thus formed is heterocyclic, it preferably contains 0, for instance one oxygen atom only. The 3- or 4- to 10-membered carbocyclic ring system may be a 3- or 4 to 7-membered monocyclic ring system, for instance, or may be a 7- to 10-membered bicyclic ring system. The ring-system may comprise a saturated carbocyclic or heterocyclic 3- to 6-membered ring fused to a benzene ring, for instance. Examples of 4- to 10-membered carbocyclic or heterocyclic ring systems thus formed include the 4 to 7-membered monocyclic heterocycloalky moieties and bicyclic heterocycloalkyl moieties set out above as examples, which may optionally be annealed to a benzene ring, as long as the ring system thus formed does not comprise more than 10 ring atoms. An exemplary embodiment of NX1X2 with R⁵¹ with R⁵⁷ forming a carbocylic ring system is octahydroindol-1-yl or 2,3-dihydro-indol-1-yl.

In preferred embodiments of compounds according to Formula I, II, III or IV, wherein X¹ and X² together with the N to which they are attached form a heterocycle according to Formula 2, Q is most preferably CR⁵⁷R⁵⁸, and at least one of R⁵¹, R⁵², R⁵³, R⁵⁴, R⁵⁵, R⁵⁶, R⁵⁷, and R⁵⁸ is selected from at least one of R⁵¹, R⁵², R⁵³, R⁵⁴, R⁵⁵, R⁵⁶, R⁵⁷, and R⁵⁸ is selected from —O—CH₃, —O—CH₂—CH₃, —O—(C₁₋₆ alkyl), —O-ALK1, —CH₂—O—CH₃, —(CH₂)₂₋₄—O—(CH₂)₀₋₄CH₃, —CH₂—S—CH₃, —OH, —CH₂—OH, —(CH₂)₂₋₄—OH, —CF₃, —CH₂—Br, —(CH₂)₂₋₄—Br, —F, —Cl, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—S(O)₂F, —C(O)—NH—(CH₂)₂—(C₆H₄)—NH—C(O)—O—C(CH₃)₃, —C(O)—NH—(CH₂)₂—(C₆H₄)—NH—C(O)H—C(—CH═CH₂, —(C₆H₄)—NH—C(O)—CH═CH₂, —(C₆H₄)—C(O)—CH═CH₂, —(C₆H₄)—CH═O, —(C₆H₄)—S(O)₂—CH═CH₂, —(C₆H₄)—F, —(C₆H₄)—S(O)₂F, —O—(CH₂)₂—(C₆H₅); —C(O)—O—(C₆F₅), —CH₂—C(O)—O—(C₆F₅), —CH═O, and allyl.

Preferably, in compounds of any of Formulas I, II, III or IV, in above Formula 2, at least one of R⁵¹, R⁵², R⁵⁵ and R⁵⁶ is selected from substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl,methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, and thiophen-3-yl. The remainder of R⁵¹, R⁵², R⁵⁵ and R⁵⁶ can then be H, for instance.

Preferably, if at least one of R⁵¹, R⁵², R⁵³, R⁵⁴, R⁵⁵, R⁵⁶, R⁵⁷, and R⁵⁸, preferably R⁵¹, R⁵², R⁵⁵ and R⁵⁶, is selected from substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, another one of R⁵¹, R⁵², R⁵³, R⁵⁴, R⁵⁵, R⁵⁶, R⁵⁷, and R⁵⁸ is selected from is preferably selected from hydroxy, C₁₋₆ alkoxy, halogen, in particular F or Cl, or oxo.

Further preferred embodiments are those wherein at least one of R⁵¹, R⁵², R⁵³, R⁵⁴, R⁵⁵, R⁵⁶, R⁵⁷, and R⁵⁸ is or comprises —(C₆H₄)—S(O)₂F, —(C₆H₄)—C(O)—CH═CH₂, —(C₆H₄)—CH═O, and —(C₆H₄)—S(O)₂—CH═CH₂.

Exemplary embodiments of moieties according to Formula 2 which may be present in compounds according to Formula I, II, III or IV are illustrated in the following by Formulas 2a to 2×3.

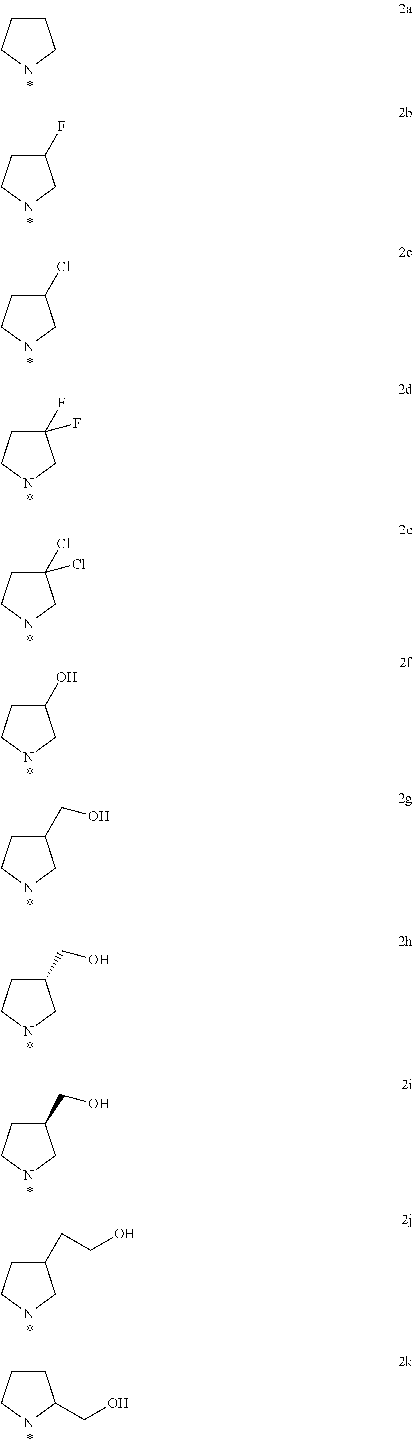

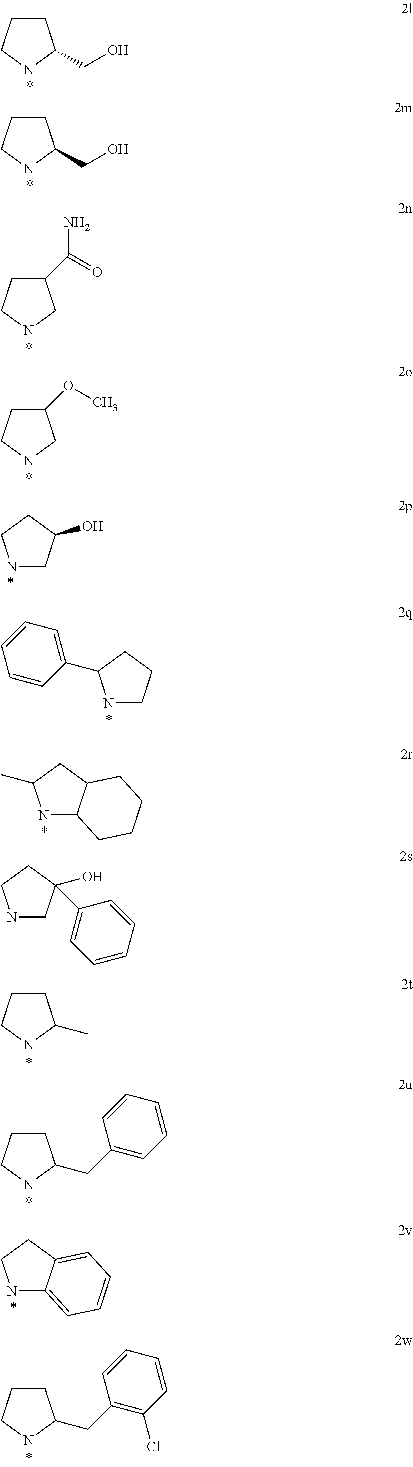

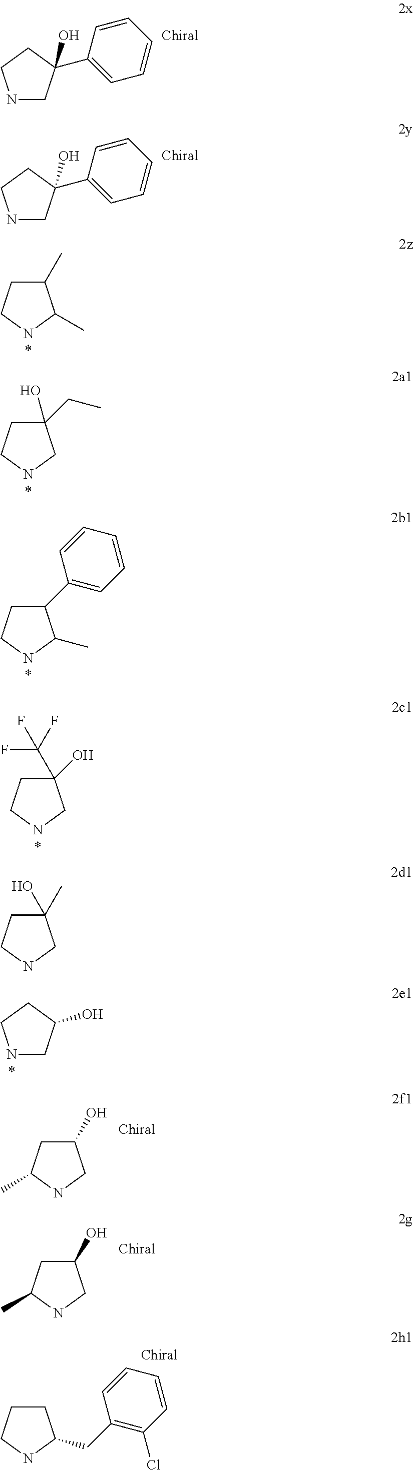

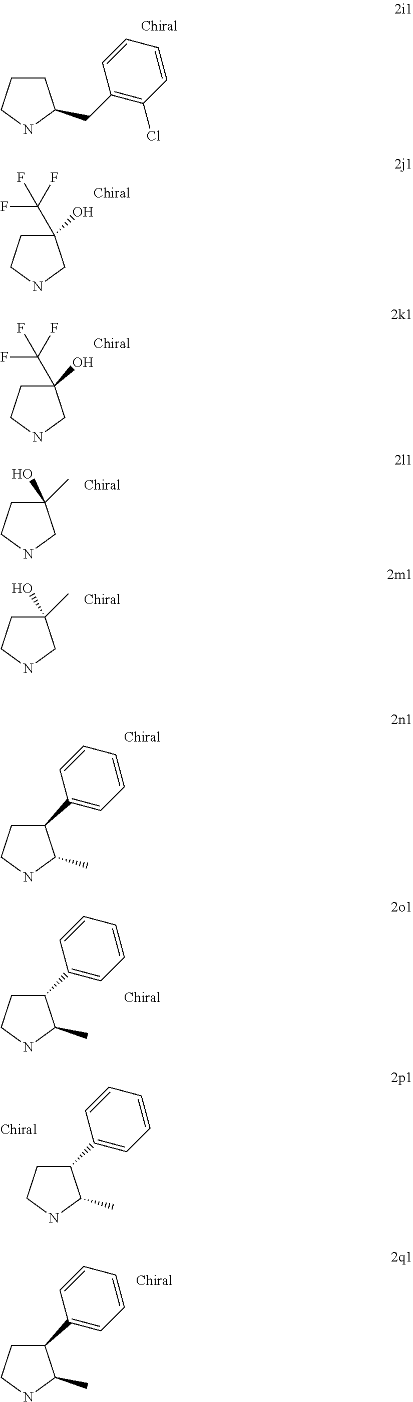

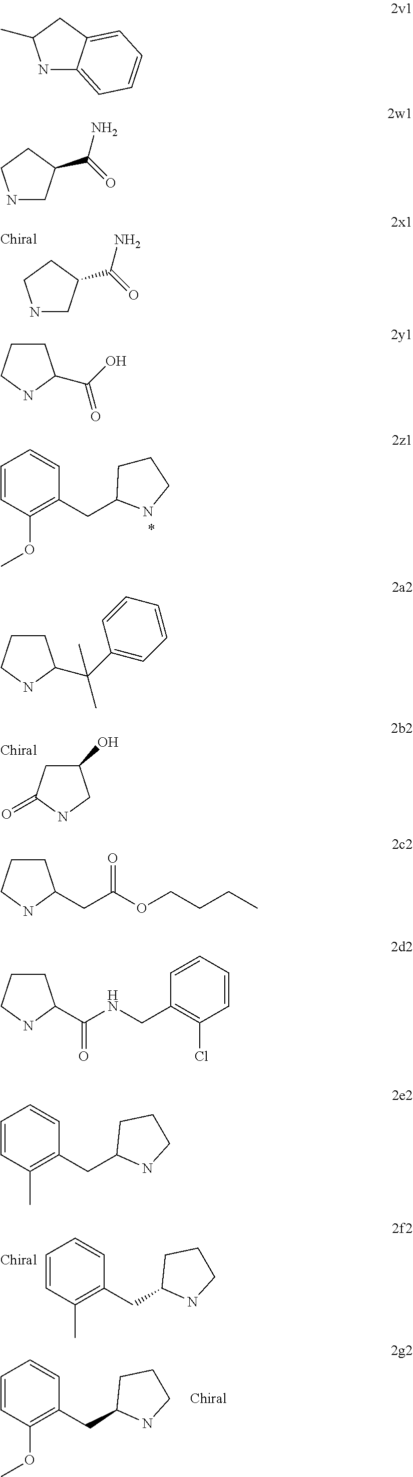

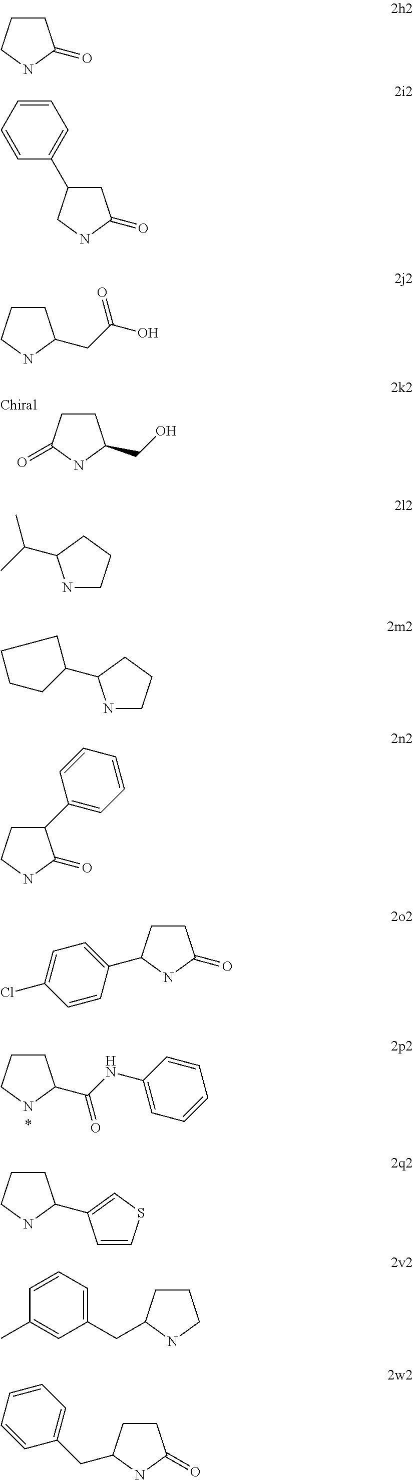

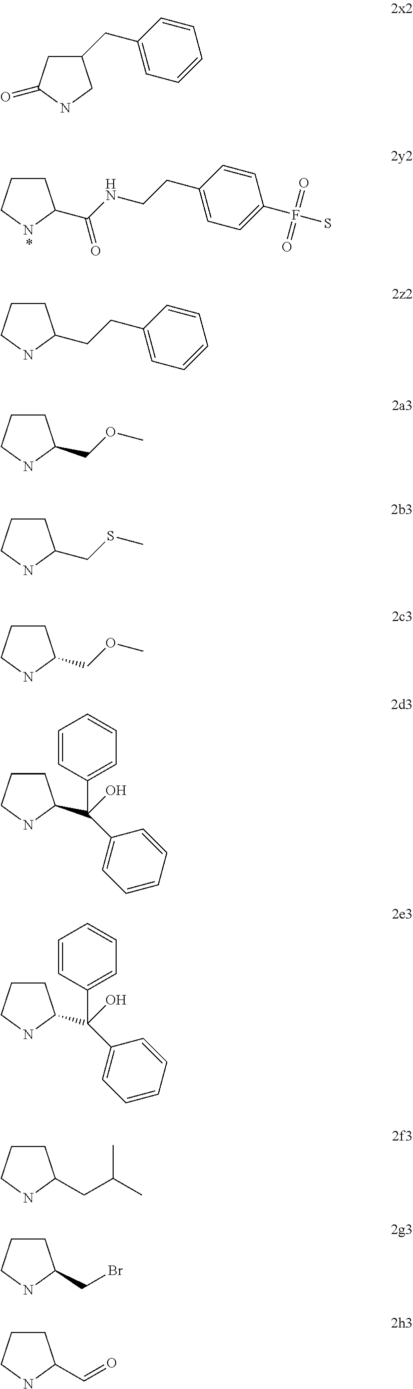

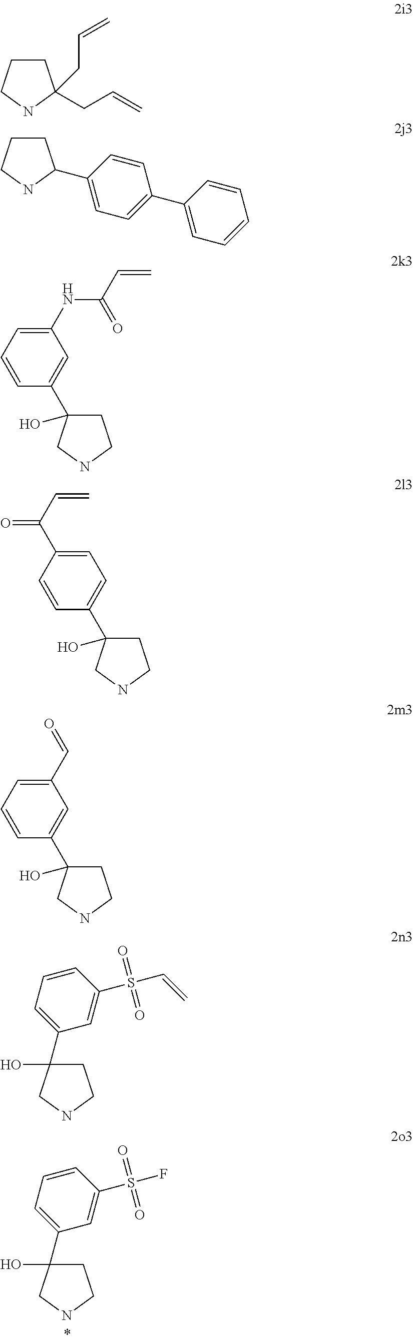

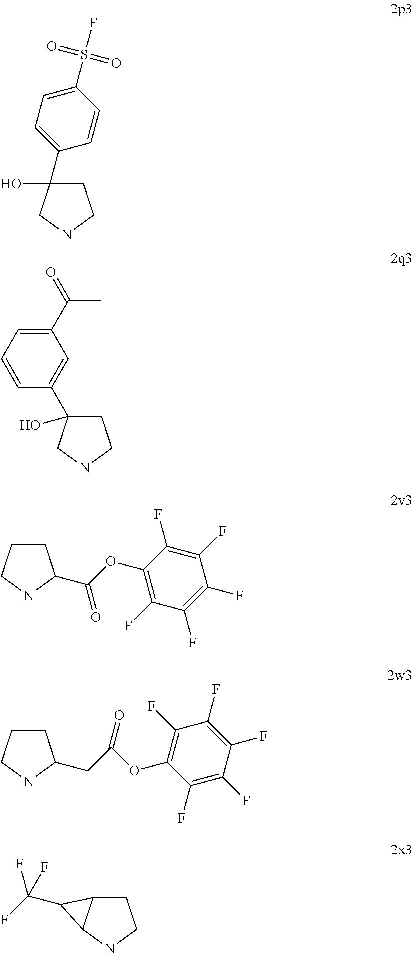

The above particular embodiments shall encompass any group according to Formula 2 that forms part of any embodiment listed below, irrespective whether it is of Formula I or II and irrespective of R¹.

In addition to the above explicit examples, pyrrolidine moieties which have the same substituent/combination of substituents in a different position respectively different positions (for instance in position 2 rather than 3 or vice versa) are equally examples of the present invention.

For instance, in Formula 2, the various substituents respectively groups and moieties are as follows:

R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁶, R⁵¹⁷, R⁵¹⁸, R⁵¹⁹, R⁵²⁰, and R⁵²¹ each being independently selected from H, ALK1 optionally substituted by one or more substituents M^{58a} and aryl optionally substituted by one or more substituents M⁵⁹; wherein R⁵¹⁹ in —S(O)₂R⁴¹⁹ may also be F or vinyl, wherein R⁵⁰¹, R⁵⁰⁵ and R⁵⁰⁸ may each independently also be vinyl,

M⁵¹, M⁵⁴ and M⁵⁵ are each independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹ and aryl optionally substituted by one or more substituents M^{59a};

M⁵² is independently selected from halogen, nitro, hydroxy, —C(O)R⁵⁰¹, —C(O)OR⁵⁰², —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, ALK1 optionally substituted by one or more substituents M^{58b}, and aryl optionally substituted by one or more substituents M^{59a};

M⁵³ is independently selected from halogen, nitro, hydroxy, —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, and ALK1 optionally substituted by one or more substituents M^{58b};

M⁵⁶ and M⁵⁷ are each independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, ALK1 optionally substituted by one or more substituents M^{58b} and aryl optionally substituted by one or more substituents M^{59a};

M^{58a} is independently selected from halogen, —CN, nitro, hydroxy, oxo (═O), —C(O)R^{501a}, —C(O)OR^{502a}, —C(O)NR^{503a}R^{504a}, —OR^{505a}, —OC(O)R^{506a}, —NR^{507a}C(O) R^{580a}, —NR^{509a}C(O)OR^{510a}, —NR^{511a}C(O)NR^{512a}R^{513a}, —NR^{516a}S(O)₂R^{517a}, —OS(O)₂R^{518a}, —S(O)ₓR^{519a}, —S(O)₂NR^{520a}R^{521a} and aryl optionally substituted by one or more substituents M^{59a}

M^{58b} is independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰9C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹;

M⁵⁹ is independently selected from halogen, nitro, hydroxy, —C(O)R^{501a}, —C(O)OR^{502a}, —C(O)NR^{503a}R^{504a}, —OR^{505a}, —OC(O) R^{506a}, —NR^{507a}C(O)R^{508a}, —NR^{509a}C(O)OR^{510a}, —NR^{511a}C(O)NR^{512a}R^{513a}, —NR^{516a}S(O)₂R^{517a}, —OS(O)₂R^{518a}, —S(O)ₓR^{519a}, —S(O)₂NR^{520a}R^{521a}, and ALK1 optionally substituted by one or more substituents M^{58b}

M^{59a} is independently selected from halogen, nitro, hydroxy, oxo (═O), C(O)R^{501a}, —C(O)OR^{5a}, —C(O)OR^{502a}, —C(O)NR^{503a}R^{504a}, —OR^{505a}, —OC(O)R^{506a}, —NR^{507a}C(O) R^{508a}, —NR^{509a}C(O)OR^{510a}, —NR^{511a}C(O)NR^{512a}R^{513a}, —NR^{516a}S(O)₂R^{517a}, —OS(O)₂R^{518a}, —S(O)ₓR^{519a}, —S(O)₂NR^{520a}R^{521a}, and ALK1, which is optionally substituted by one or more of halogen, —CN, nitro, hydroxy or C₁₋₁₂ alkoxy;

wherein R^{501a}, R^{502a}, R^{503a}, R^{504a}, R^{550a}, R^{506a}, R^{507a}, R^{508a}, R^{509a}, R^{510a}, R^{511a}, R^{512a}, R^{513a}, R^{516a}, R^{517a}, R^{518a}, R^{519a}, R^{520a}, and R^{521a} are each independently selected from H, ALK1 optionally substituted by one or more substituents M^{58c} and aryl optionally substituted by one or more substituents M^{59b}, wherein R^{519a} in —S(O)₂R^{419a} may also be F or vinyl, and wherein R^{501a}, R^{550a}, and R^{508a} may each independently also be vinyl,

M^{58c} is independently selected from halogen, —CN, nitro, hydroxy, oxo, —C(O)R^{501b}, —C(O)OR^{502b}, —C(O)NR^{503b}R^{504b}, —OR^{505b}, —OC(O)R^{506b}, —NR^{507b}C(O) R^{508b}, —NR^{509b}C(O)OR^{510b}, —NR^{511b}C(O)NR^{512b}R^{513b}, —NR^{516b}S(O)₂R^{517b}, —OS(O)₂R^{518b}, —S(O)ₓR^{519b}, —S(O)₂NR^{520b}R^{521b}, and aryl optionally substituted by one or more substituents M^{59b}

M^{59b} is independently selected from halogen, —CN, nitro, hydroxy, oxo, —C(O)R^{501b}, —C(O)OR^{502b}, —C(O)NR^{503b}R^{504b}, —OR^{505b}, —OC(O)R^{506b}, —NR^{507b}C(O) R^{508b}, —NR^{509b}C(O)OR^{510b}, —NR^{511b}C(O)NR^{512b}R^{513b}, —NR^{516b}S(O)₂R^{517b}, —OS(O)₂R^{518b}, —S(O)ₓR^{519b}, —S(O)₂NR^{520b}R^{521b}

wherein R^{501b}, R^{502b}, R^{503b}, R^{504b}, R^{505b}, R^{506b}, R^{507b}, R^{508b}, R^{509b}, R^{510b}, R^{511b}, R^{512b}, R^{513b}, R^{516b}, R^{517b}, R^{518b}, R^{519b}, R^{520b}, and R^{521b} are each independently selected from H, ALK1 optionally substituted by halogen, —CN, nitro, hydroxy, oxo and aryl optionally substituted halogen, —CN, nitro, or hydroxy, wherein R^{519a} in —S(O)₂R^{419a} may also be F or vinyl, and wherein R^{501a}, R^{505a} and R^{508a} may each independently also be vinyl.

The same substitution pattern as above (designation of M and R variables) is also applicable for the M and R groups and moieties of Formulas 1, 2 and 4.

The present invention thus encompasses the following exemplary embodiment of compounds according to Formula I, respectively groups of compounds:

| R¹ | NX¹X² |
|---|---|
| Opt. subst. C₁-C₆ alkyl | Formula 2 |
| Opt. subst. C₁-C₆ alkyl | 2a-2x3 |
| Opt. subst. C₃ or C₄ cycloalkyl | Formula 2 |
| Opt. subst. C₃ or C₄ cycloalkyl | 2a-2x3 |
| Opt. subst. C₃ or C₄ cycloalkyl | Formula 2 |
| Opt. subst. C₃ or C₄ cycloalkyl | 2a-2x3 |
| —CH₃ | Formula 2 |
| —CH₃ | 2a-2x3 |
| —C₂H₅ | Formula 2 |
| —C₂H₅ | 2a-2x3 |
| Cyclopropyl | Formula 2 |
| Cyclopropyl | 2a-2x3 |
| isopropyl | Formula 2 |
| isopropyl | 2a-2x3 |
| cylopropyl-methyl- | Formula 2 |
| cylopropyl-methyl- | 2a-2x3 |
| cyclopentyl-methyl- | Formula 2 |
| cyclopentyl-methyl- | 2a-2x3 |
| ALK 1, substituted with unsubstituted or substituted phenyl | Formula 2 |
| ALK 1, substituted with unsubstituted or substituted phenyl | 2a-2x3 |
| —C₂H_{4—}(unsubstituted or substituted phenyl) | Formula 2 |
| —C₂H_{4—}(unsubstituted or substituted phenyl) | 2a-2x3 |

In certain alternative embodiments according to the present invention, in each and any compound according to one of above Formulas I, II, III, IV, X¹ and X² together with the N to which they are attached may form a heterocycle according to Formula 3:

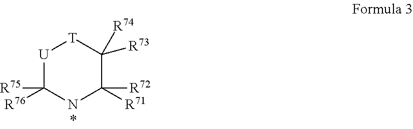

wherein
U is selected from CR⁷⁷R⁷⁸, O and S;
T is selected from CR⁸⁰R⁸¹, O, and S, with the proviso that only one of U and T may be selected from O and S; and
R⁷¹, R⁷², R⁷³, R⁷⁴, R⁷⁵, R⁷⁶, R⁷⁷, R⁷⁸, R⁸⁰ and R⁸¹ are independently selected from H, hydroxy, nitro, —CN, halogen, ALK1 optionally substituted by one or more substituents M⁷¹, aryl optionally substituted by one or more substituents M⁷², heterocyclyl optionally substituted by one or more substituents M⁷³, ALK2 optionally substituted by one or more substituents M⁷⁴, ALK3 optionally substituted by one or more substituents M⁷⁵, —C(O)R⁷⁰¹, —C(O)OR⁷⁰², —C(O)NR⁷⁰³R⁷⁰⁴, —OR⁷⁰⁵, —OC(O) R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, and —S(O)₂NR⁷²⁰R⁷²¹;
or a combination of R⁷¹ and R⁷², R⁷³ and R⁷⁴, R⁷⁵ and R⁷⁶, R⁷⁷ and R⁷⁸, or R⁸⁰ and R⁸¹ together form ═O or ═S,
or a combination of R⁷¹ and R⁷², R⁷³ and R⁷⁴, R⁷⁵ and R⁷⁶, R⁷⁷ and R⁷⁸, or R⁸⁰ and R⁸¹ together with the C atom to which they are attached form a 4- to 10-membered carbocyclic or heterocyclic ring system, which ring system is optionally substituted by one or more substituents M⁷⁶,
or a combination of R⁷² and R⁷⁴, R⁷⁴ and R⁸⁰, R⁸⁰ and R⁷⁸, or R⁷⁸ and R⁷⁶ together with the C atoms to which they are attached form a 3- or 4- to 10-membered carbocyclic or heterocyclic ring system, which ring system is optionally substituted by one or more substituents M⁷⁷,
R⁷⁰¹, R⁷⁰², R⁷⁰³, R⁷⁰⁴, R⁷⁰⁵, R⁷⁰⁶, R⁷⁰⁷, R⁷⁰⁸, R⁷⁰⁹, R⁷¹⁰, R⁷¹¹, R⁷¹², R⁷¹³, R⁷¹⁶, R⁷¹⁷, R⁷¹⁸, R⁷¹⁹, R⁷²⁰ and R⁷²¹ are independently selected from H, ALK1 optionally substituted by one or more substituents M^{78a} and aryl optionally substituted by one or more substituents M⁷⁹;
wherein R⁷¹⁹ in —S(O)₂R⁷¹⁹ may also be F or vinyl,
wherein R⁷⁰¹, R⁷⁰⁵ and R⁷⁰⁸ may each independently also be vinyl,
M⁷¹, M⁷⁴, M⁷⁵ and M^{78a} are each independently selected from hydroxy, oxo (═O), nitro, —CN, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and aryl optionally substituted by one or more substituents M^{79a};
M⁷² each independently selected from hydroxy, nitro, halogen, —C(O)R⁷⁰¹, —C(O)OR⁷⁰², —OR⁷⁰⁵, —OC(O)R⁷⁰⁶, —NR⁷⁰⁷C(O) R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹, ALK1 optionally substituted by one or more substituents M^{78b} and aryl optionally substituted by one or more substituents M^{79a};
M⁷³ and M⁷⁹ each independently selected from hydroxy, nitro, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and ALK1 optionally substituted by one or more substituents M^{78b};
M⁷⁶ and M⁷⁷ each independently selected from hydroxy, oxo (═O), nitro, —CN, halogen, —C(O) R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹, ALK1 optionally substituted by one or more substituents M^{78b} and aryl optionally substituted by one or more substituents M^{79a};
M^{78b} each independently selected from hydroxy, oxo (═O), nitro, —CN, halogen, —C(O) R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, and —S(O)₂NR⁷²⁰R⁷²¹;
M^{79a} each independently selected from hydroxy, oxo (═O), nitro, halogen, —C(O)R⁷⁰¹, —C(O)OR⁷⁰², —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and ALK1, which is optionally substituted by one or more of halogen, —CN, nitro, hydroxy or C₁₋₁₂ alkoxy;
with the proviso that any N-atom, if present, in addition to the N-atom depicted in above Formula 3 is comprised in the form of a substituent selected from nitro, —CN, —C(O)NR⁷⁰³R⁷⁰⁴, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷ and —S(O)₂NR⁷²⁰R⁷²¹

As per the general definition, x may be 0, 1 or 2; and is preferably 2.

Formula 3 thus covers, for instance, the following moieties:

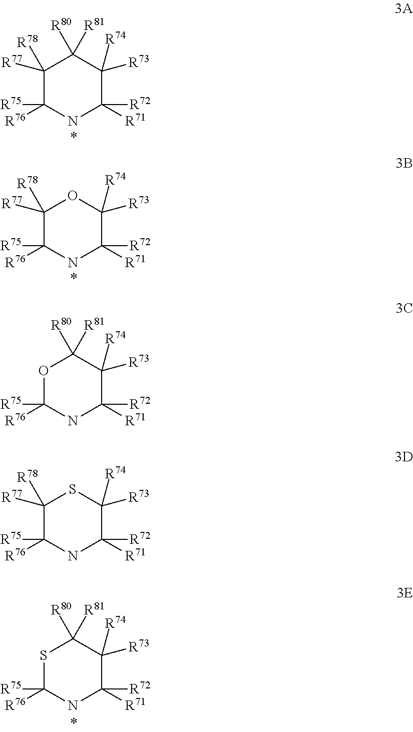

Most preferably, X¹ and X² together with the N to which they are attached form a 6-membered ring according to formula 3A.

In certain other preferred embodiments, X¹ and X² together with the N to which they are attached form a 6-membered ring according to formula 3B.

According to the proviso, the moieties according to Formula 3 do not comprise any amino groups or N ring members in any heterocyclic ring formed by the substituents.

In certain exemplary embodiments, U is selected from CR⁷⁷R⁷⁸, O and S; and T is selected from CR⁸⁰R⁸¹, O, and S, with the proviso that only one of U and T may be selected from O and S; and wherein the N atom depicted in above Formula 3 is the only N atom comprised by the moiety defined by Formula 3.

In preferred embodiments, the moiety according to Formula 3 may be mono-, di- or trisubstituted, i.e. one, two or three of R⁷¹, R⁷², R⁷³, R⁷⁴, R⁷⁵, R⁷⁶, R⁷⁷, R⁷⁸, R⁸⁰ and R⁸¹ are different from H. In preferred embodiments of compounds with moieties according to Formula 3 for use in compounds according to reach of Formulas I, II, III and IV, such as 3A or 3B, most preferably 3A, at least one of R⁷¹, R⁷², R⁷³, R⁷⁴, R⁷⁵, R⁷⁶, R⁷⁷, R⁷⁸, R⁸⁰ and R⁸¹ is selected from —O—CH₃, —O—CH₂—CH₃, —O—(C₁₋₆ alkyl), —O-ALK1, —CH₂—O—CH₃, —(CH₂)₂₋₄—O—(CH₂)₀₋₄CH₃, —CH₂—S—CH₃, —OH, —CH₂—OH, —(CH₂)₂₋₄—OH, —CF₃, —CH₂—Br, —(CH₂)₂₋₄—Br, —F, —Cl, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—S(O)₂F, —C(O)—NH—(CH₂)₂—(C₆H₄)—NH—C(O)—O—C(CH₃)₃, —C(O)—NH—(CH₂)₂—(C₆H₄)—NH—C(O)H—C(—CH═CH₂, —(C₆H₄)—NH—C(O)—CH═CH₂, (C₆H₄)—C(O)—CH═CH₂, —(C₆H₄)—CH═O, —(C₆H₄)—S(O)₂—CH═CH₂, —(C₆H₄)—F, —(C₆H₄)—S(O)₂F, —O—(CH₂)₂—(C₆H₅); —C(O)—O—(C₆F₅), —CH₂—C(O)—O—(C₆F₅), —CH═O, and allyl. The remainder of R⁷¹, R⁷², R⁷³, R⁷⁴, R⁷⁵, R⁷⁶, R⁷⁷, R⁷⁸, R⁸⁰ and R⁸¹ would typically be H, as evident from the examples herein,

In certain preferred embodiments of compounds with moieties according to Formula 3 for use in compounds according to reach of Formulas I, II, III and IV, such as 3A or 3B, most preferably 3A, at least one of R⁷¹, R⁷², R⁷³, R⁷⁴, R⁷⁵, R⁷⁶, R⁷⁷, R⁷⁸, R⁸⁰ and R⁸¹, is selected from unsubstituted phenyl or phenyl substituted with one or more of halogen, preferably F and/or Cl, -hydroxy, C₁-C₀₆ alkoxy, methoxy, C₁-C₀₆ haloalkoxy, —S(O)₂F, —S(O)₂CH═CH₂, —NH—C(O)—CH═CH₂, —C(O)—CH═CH₂, and —CH(═O); unsubstituted benzyl or benzyl_{[FB4]} substituted with one or more of halogen, preferably F and/or Cl, -hydroxy, C₁-C₆ alkoxy, methoxy, C₁-C₆ haloalkoxy, —S(O)₂F, —S(O)₂CH═CH₂, —NH—C(O)—CH═CH₂, —C(O)—CH═CH₂, and —CH(═O); or unsubstituted phenylethyl or phenethyl substituted with one or more of halogen, preferably F and/or Cl, -hydroxy, methoxy, C₁-C₆ alkoxy, C₁-C₆ haloalkoxy, —S(O)₂F, —S(O)₂CH═CH₂, —NH—C(O)—CH═CH₂, —C(O)—CH═CH₂, and —CH(═O).

In some embodiments, at least one of R⁷¹, R⁷², R⁷⁵ and R⁷⁶ is selected from: unsubstituted phenyl or phenyl substituted with one or more of halogen, preferably F and/or Cl, -hydroxy, C₁-C₆ alkoxy, methoxy, C₁-C₆ haloalkoxy, —S(O)₂F, —S(O)₂CH═CH₂, —NH—C(O)—CH═CH₂, —C(O)—CH═CH₂, and —CH(═O); unsubstituted benzyl or benzyl_{[FB5]} substituted with one or more of halogen, preferably F and/or Cl, -hydroxy, C₁-C₆ alkoxy, methoxy, C₁-C₆ haloalkoxy, —S(O)₂F, —S(O)₂CH═CH₂, —NH—C(O)—CH═CH₂, —C(O)—CH═CH₂, and —CH(═O); or unsubstituted phenylethyl or phenethyl substituted with one or more of halogen, preferably F and/or Cl, -hydroxy, methoxy, C₁-C₆ alkoxy, C₁-C₆ haloalkoxy, —S(O)₂F, —S(O)₂CH═CH₂, —NH—C(O)—CH═CH₂, —C(O)—CH═CH₂, and —CH(═O). Embodiments with substituted benzyl, in particular benzyl_{[FB6]} substituted with one or more of halogen, preferably F and/or Cl, -hydroxy, C₁-C₆ alkoxy, methoxy, C₁-C₆ haloalkoxy are preferred.

In those embodiments, wherein at least one of R⁵, R⁵², R⁵³, R⁵⁴, R⁵⁵, R⁵⁶, R⁵⁷, and R⁵⁸ is selected from optionally substituted phenyl, benzyl or phenethyl, a least another one of R⁵¹, R⁵², R⁵³, R⁵⁴, R⁵⁵, R⁵⁶, R⁵⁷, and R⁵⁸ is preferably selected from hydroxy, C₁₋₆ alkoxy, halogen, in particular F or Cl, or oxo.

In preferred embodiments of compounds with moieties according to Formula 3 for use in compounds according to reach of Formulas I, II, III and IV, such as 3A or 3B, at least one of R⁷¹, R⁷², R⁷³, R⁷⁴, R⁷⁵, R⁷⁶, R⁷⁷, R⁷⁸, R⁸⁰ and R⁸¹ is selected from —O—CH₃, —CH₂—O—CH₃, —CH₂—S—CH₃, —OH, —CH₂—OH, —CF₃, —CH₂—Br, F, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—SO₂F, —C(O)—NH—(CH₂)₂—(C₆H₄)—NH—C(O)—O—C(CH₃)₃, —C(O)—NH—(CH₂)₂—(C₆H₄)—NH—C(O)—CH═CH₂, —(C₆H₄)—NH—C(O)—CH═CH₂, —(C₆H₄)—C(O)—CH═CH₂, —(C₆H₄)—CH═O, —(C₆H₄)—S(O)₂—CH═CH₂, —(C₆H₄)—F, —O—(CH₂)₂—(C₆H₅); —C(O)—O—(C₆F₅), —CH₂—C(O)—O—(C₆F₅), —CH═O, and allyl. Preferably, if at least one of R⁷¹, R⁷², R⁷³, R⁷⁴, R⁷⁵, R⁷⁶, R⁷⁷, R⁷⁸, R⁸⁰ and R⁸¹ is selected from substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, another one of R⁷¹, R⁷², R⁷³, R⁷⁴, R⁷⁵, R⁷⁶, R⁷⁷, R⁷⁸, R⁸⁰ and R⁸¹ is selected from is preferably selected from hydroxy, C₁₋₆ alkoxy, halogen, in particular F or Cl, or oxo. In those embodiments, the remainder of R⁷¹, R⁷², R⁷³, R⁷⁴, R⁷⁵, R⁷⁶, R⁷⁷, R⁷⁸, R⁸⁰ and R⁸¹ are preferably H.

Exemplary embodiments of moieties according to Formula 3 for use in compounds according to each of Formulas I, II, III and IV are illustrated in the following by Formulas 3Ba and 3Aa to 3Ac1:

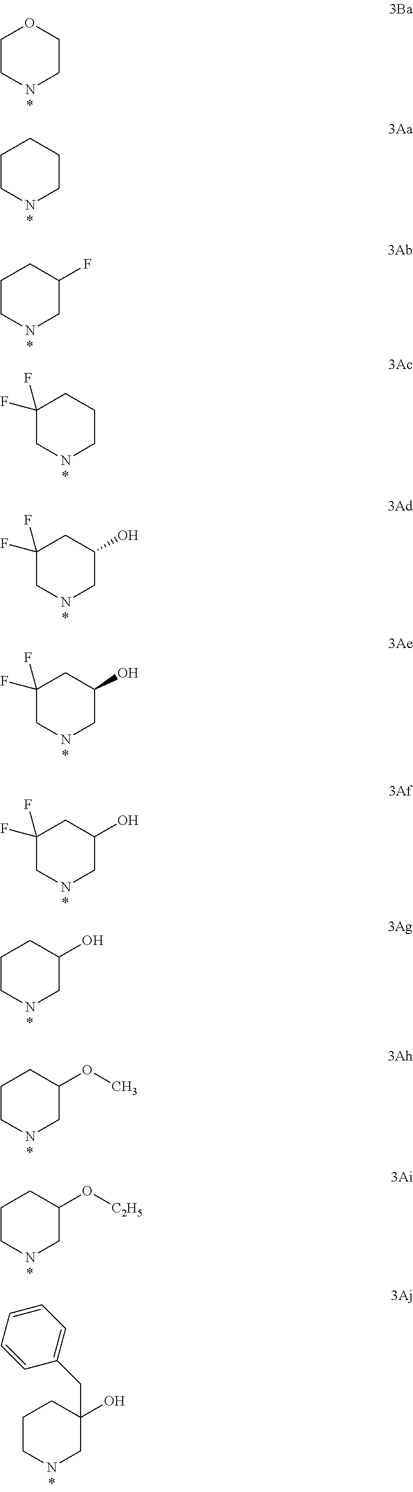

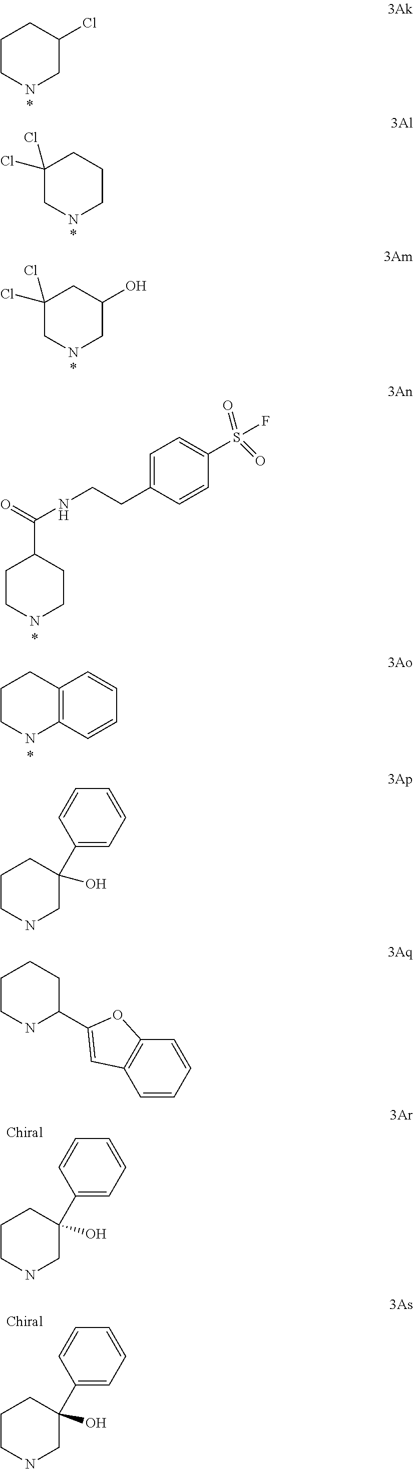

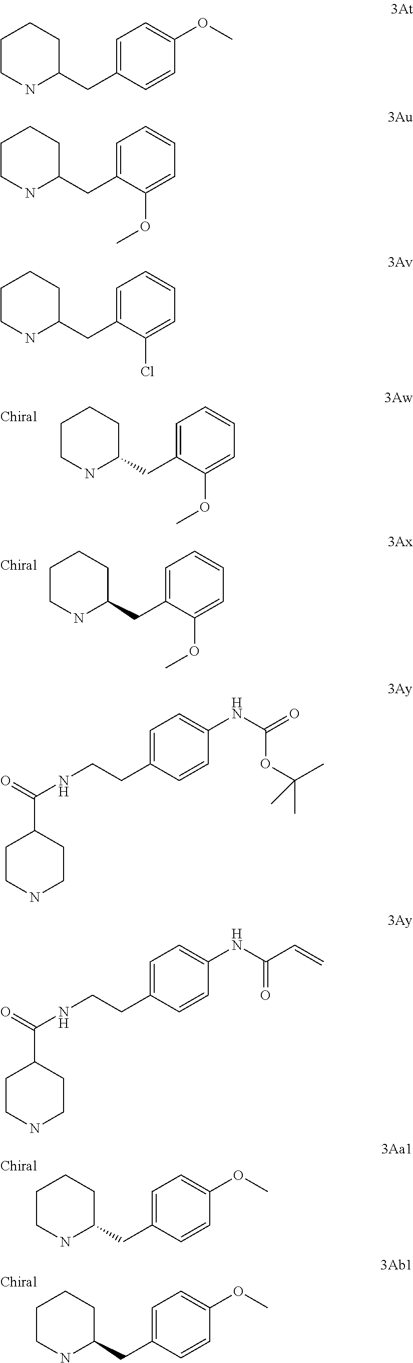

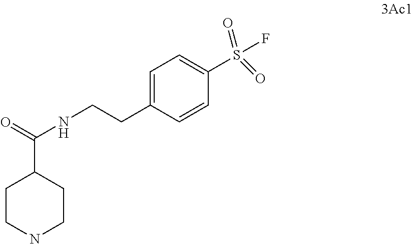 [SMILES: O=C(NCCc1ccc(S(=O)(=O)F)cc1)C1CCNCC1]

The present invention thus encompasses the following exemplary compounds according to Formula I, respectively groups of compounds, having NX¹X² corresponding to Formula 3:

| R1 | NX1X2 |
|---|---|
| Opt. subst. C1-C6 alkyl | Formula 3 |
| Opt. subst. C1-C6 alkyl | Formulas 3Ba and 3Aa to 3Ac1 |
| Opt. subst. C3 or C4 cycloalkyl | Formula 3 |
| Opt. subst. C3 or C4 cycloalkyl | Formulas 3Ba and 3Aa to 3Ac1 |
| Opt. subst. C3 or C4 cycloalkyl | Formula 3 |
| Opt. subst. C3 or C4 cycloalkyl | Formulas 3Ba and 3Aa to 3Ac1 |
| —CH3 | Formula 3 |
| —CH3 | Formulas 3Ba and 3Aa to 3Ac1 |
| —C2H5 | Formula 3 |
| —C2H5 | Formulas 3Ba and 3Aa to 3Ac1 |
| Cyclopropyl | Formula 3 |
| Cyclopropyl | Formulas 3Ba and 3Aa to 3Ac1 |
| isopropyl | Formula 3 |
| isopropyl | Formulas 3Ba and 3Aa to 3Ac1 |
| cylopropyl-methyl- | Formula 3 |
| cylopropyl-methyl- | Formulas 3Ba and 3Aa to 3Ac1 |
| cyclopentyl-methyl- | Formula 3 |
| cyclopentyl-methyl- | Formulas 3Ba and 3Aa to 3Ac1 |
| ALK 1, substituted with unsubstituted | Formula 3 |
| or substituted phenyl | |
| ALK 1, substituted with unsubstituted | Formulas 3Ba and 3Aa to 3Ac1 |
| or substituted phenyl | |
| —C2H4—(unsubstituted or substituted | Formula 3 |
| phenyl) | |
| —C2H4—(unsubstituted or substituted | Formulas 3Ba and 3Aa to 3Ac1 |
| phenyl) | |

In certain alternative embodiments, in any compound according to one of above Formulas I, II, III, and IV, X¹ and X² together with the N to which they are attached may form a heterocycle according to Formula 4

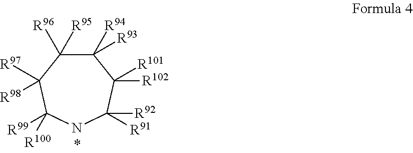

wherein
R⁹¹, R⁹², R⁹³, R⁹⁴, R⁹⁵, R⁹⁶, R⁹⁷, R⁹⁸, R⁹⁹, R¹⁰⁰, R¹⁰¹ and R¹⁰² are independently selected from H, hydroxy, nitro, —CN, halogen, ALK1 optionally substituted by one or more substituents M⁹¹, aryl optionally substituted by one or more substituents M⁹², heterocyclyl optionally substituted by one or more substituents M⁹³, ALK2 optionally substituted by one or more substituents M⁹⁴, ALK3 optionally substituted by one or more substituents M⁹⁵, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹;
or a combination of R⁹¹ and R⁹², R⁹³ and R⁹⁴, R⁹⁵ and R⁹⁶, R⁹⁷ and R⁹⁸, R⁹⁹ and R¹⁰⁰, or R¹⁰¹ and R¹⁰² together forms ═O or ═S,
or R¹⁰¹ and R⁹⁷ together form an oxygen bridge member (—O—),
or a combination of R⁹¹ and R⁹², R⁹³ and R⁹⁴, R⁹⁵ and R⁹⁶, R⁹⁷ and R⁹⁸, or R⁹⁹ and R¹⁰⁰ together with the C atom to which they are attached form a 4- to 10-membered carbocyclic or heterocyclic ring system, which ring system is optionally substituted by one or more substituents M⁹⁶,
or a combination of R⁹¹ and R¹⁰¹, R⁹³ and R¹⁰¹, R⁹³ and R⁹⁵, R⁹⁵ and R⁹⁷, R⁹⁷ and R⁹⁹ together with the C atoms to which they are attached form a 3- or 4- to 10-membered carbocyclic or heterocyclic ring system, which ring system is optionally substituted by one or more substituents M⁹⁷,
R⁹⁰¹, R⁹⁰², R⁹⁰³, R⁹⁰⁴, R⁹⁰⁵, R⁹⁰⁶, R⁹⁰⁷, R⁹⁰⁸, R⁹⁰⁹, R⁹¹⁰, R⁹¹¹, R⁹¹², R⁹¹³, R⁹¹⁶, R⁹¹⁷, R⁹¹⁸, R⁹¹⁹, R⁹²⁰ and R⁹²¹ are each independently selected from H, ALK1 optionally substituted by one or more substituents M^{98a} and aryl optionally substituted by one or more substituents M⁹⁹;
wherein R⁹¹⁹ in —S(O)₂R⁹¹⁹ may also be F or vinyl,
wherein R⁹⁰¹, R⁹⁰⁵ and R⁹⁰⁸ may each independently also be vinyl,
M⁹¹, M⁹⁴, M⁹⁵ and M^{98a} are each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O) R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and aryl optionally substituted by one or more substituents M^{99a};
M⁹² is each independently selected from hydroxy, nitro, halogen, —C(O)R⁹⁰¹, —C(O)OR⁹⁰², —OR⁹⁰⁵, —OC(O) R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1 optionally substituted by one or more substituents M^{98b};
M⁹³ and M⁹⁹ are each independently selected from hydroxy, nitro, halogen, —C(O)R⁹⁰¹, —C(O)OR⁹⁰², —C(O)NR⁹⁰³R⁹⁰⁴, —OR⁹⁰⁵, —OC(O) R⁹⁰⁶, —NR⁹⁰7C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1 optionally substituted by one or more substituents M^{98b};
M⁹⁶ and M⁹⁷ are each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹, ALK1 optionally substituted by one or more substituents M^{98b} and aryl optionally substituted by one or more substituents M^{99a};
M^{98b} each independently selected from hydroxy, oxo (═O), nitro, —CN, halogen, —C(O) R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰9C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹,
M^{99a} each independently selected from hydroxy, oxo (═O), nitro, halogen, —C(O)R⁹⁰¹, —C(O)OR⁹⁰², —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1, which is optionally substituted by one or more of halogen, —CN, nitro, hydroxy or C₁₋₁₂ alkoxy,
with the proviso that any N-atom, if present, in addition to the N-atom depicted in above Formula 4 is comprised in the form of a substituent selected from nitro, —CN, —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷ and —S(O)₂NR⁹²⁰R⁹²¹.

In harmony with the proviso, V is CR¹⁰¹R¹⁰², and any N-atom, if present, in addition to the N-atom depicted in above Formula 4 is comprised in the form of a substituent selected from nitro, —CN, —C(O)NR⁹⁰³R⁹⁰⁴, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷ and S(O)₂NR⁹²⁰R⁹²¹, more preferably —C(O)NR⁹⁰³R⁹⁰⁴, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷ and —S(O)₂NR⁹²⁰R⁹²¹.

In further exemplary embodiments, V is CR¹⁰¹R¹⁰², and the N atom depicted in above Formula 4 is the only N atom comprised by Formula 4.

An example of a moiety according to Formula 4 which can be used in any of the above mentioned compounds according to Formulas I, II, III or IV is the following:

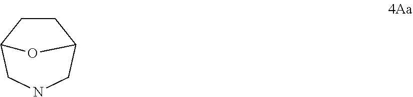

Preferably, at least one of R⁹¹, R⁹², R⁹³, R⁹⁴, R⁹⁵, R⁹⁶, R⁹⁷, R⁹⁸, R⁹⁹, R¹⁰⁰, R¹⁰¹ and R¹⁰² is selected from —O—CH₃, —O—CH₂—CH₃, —O—(C₁₋₆ alkyl), —O-ALK1, —CH₂—O—CH₃, —(CH₂)₂₋₄—O—(CH₂)₀₋₄CH₃, —CH₂—S—CH₃, —OH, —CH₂—OH, —(CH₂)₂₋₄—OH, —CF₃, —CH₂—Br, —(CH₂)₂₋₄—Br, —F, —Cl, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—S(O)₂F, —C(O)—NH—(CH₂)₂—(C₆H₄)—NH—C(O)—O—C(CH₃)₃, —C(O)—NH—(CH₂)₂—(C₆H₄)—NH—C(O)—CH═CH₂, —(C₆H₄)—NH—C(O)—CH═CH₂, (C₆H₄)—C(O)—CH═CH₂, —(C₆H₄)—CH═O, —(C₆H₄)—S(O)₂—CH═CH₂, —(C₆H₄)—F, —(C₆H₄)—S(O)₂F, —O—(CH₂)₂—(C₆H₅); —C(O)—O—(C₆F₅), —CH₂—C(O)—O—(C₆F₅), —CH═O, and allyl. Preferably, the remainder of R⁹¹, R⁹², R⁹³, R⁹⁴, R⁹⁵, R⁹⁶, R⁹⁷, R⁹⁸, R⁹⁹, R¹⁰⁰, R¹⁰¹ and R¹⁰² are H.

Preferably, in a compound according to Formula I, II, III or IV, X¹ and X² together with the N to which they are attached form a heterocycle which is selected from:
azetidin-1-yl, 3-fluoro-azetidin-1-yl, 3-oxo-azetidin-1-yl, 3-chloro-azetidin-1-yl, 3-hydroxy-azetidin-1-yl, 2-(4-fluoro-phenyl)-azetidin-1-yl, 2-(4-chloro-phenyl)-azetidin-1-yl, 1-oxa-5-azaspiro[3.3]heptyl, 3-(N-(2-(4-fluorosulfony-phenyl)-ethyl)-amino-carbonyl)-azetidin-1-yl, pyrrolidin-1-yl, 3-hydroxymethyl-pyrrolidin-1-yl, (S)-3-hydroxymethyl-pyrrolidin-1-yl, (R)-3-hydroxymethyl-pyrrolidin-1-yl, 3-hydroxyethyl-pyrrolidin-1-yl, 3,3-difluoro-pyrrolidin-1-yl, 3-methoxy-pyrrolidin-1-yl, 3-ethoxy-pyrrolidin-1-yl, 3-hydroxy-pyrrolidin-1-yl, (R)-2-hydroxymethyl-pyrrolidin-1-yl, (S)-2-hydroxymethyl-pyrrolidin-1-yl, 2-Isopropyl-pyrrolidin-1-yl, 2-isobutyl-pyrrolidin-1-yl, (2S)-2-(bromomethyl)pyrrolidin-1-yl, 2-phenyl-pyrrolidin-1-yl, 2-benzyl-pyrrolidin-1-yl, 2-methyl-3-phenyl-pyrrolidin-1-yl, 3-hydroxy-3-phenyl-pyrrolidin-1-yl, 2-((S)-diphenyl-hydroxy-methyl)-pyrrolidin-1-yl, 2-((R)-diphenyl-hydroxy-methyl)-pyrrolidin-1-yl, 2-(2-methoxy-benzyl)-pyrrolidin-1-yl, (S)-2-(2-methoxy-benzyl)-pyrrolidin-1-yl, (R)-2-(2-methoxy-benzyl)-pyrrolidin-1-yl, 2-(1-methyl-1-phenyl-ethyl)-pyrrolidin-1-yl, 2-(2-methyl-benzyl)-pyrrolidin-1-yl, 2-(3-methyl-benzyl)-pyrrolidin-1-yl 2-(2-chloro-benzyl)-pyrrolidin-1-yl, 2-(4-chloro-benzyl)-pyrrolidin-1-yl, 2-methyl-pyrrolidin-1-yl, 2,3-dimethyl-pyrrolidin-1-yl, 3-ethyl-3-hydroxy-pyrrolidin-1-yl, 3-hydroxy-3-methyl-pyrrolidin-1-yl, 3-hydroxy-5-methyl-pyrrolidin-1-yl, 3-hydroxy-3-trifluoromethyl-pyrrolidin-1-yl, 2-(3-chloro-benzyl)-pyrrolidin-1-yl, 3-trifluoromethyl-pyrrolidin-1-yl, 3-carbamoyl-pyrrolidin-1-yl, (S)-3-carbamoyl-pyrrolidin-1-yl, (R)-3-carbamoyl-pyrrolidin-1-yl, 2-methyl-octahydro-indol-1-yl, 2,3-dihydro-indol-1-yl, 2-(2-chloro-benzyl)-pyrrolidin-1-yl, 2-methyl-3-phenyl-pyrrolidin-1-yl, (2S,3R)-2-methyl-3-phenyl-pyrrolidin-1-yl, (2S,3S)-2-methyl-3-phenyl-pyrrolidin-1-yl, (2R,3S)-2-methyl-3-phenyl-pyrrolidin-1-yl, (2R,3R)-2-methyl-3-phenyl-pyrrolidin-1-yl, 1-pyrrolidin-2-yl-acetic acid butyl ester, 1-pyrrolidine-2-carboxylic acid 2-chloro-benzylamide, 1-pyrrolidin-2-yl-acetic acid, (S)-5-hydroxymethyl-2-oxo-pyrrolidin-1 yl, 2-Cyclopentyl-pyrrolidin-1-yl, 3-phenyl-2-oxo-pyrrolidin-1-yl, 5-(4-chloro-phenyl)-2-oxo-pyrrolidin1-yl, 2-(N-phenylaminocarbonyl)-pyrrolidin1-yl, 2-thiophen-3-yl-pyrrolidin-1-yl, 5-benzyl-2-oxo-pyrrolidin-1-yl, 4-benzyl-2-oxo-pyrrolidin-1-yl, 2-(2-phenylethyl)pyrrolidin-1-yl, (2S)-2-(methoxymethyl)pyrrolidin-1-yl, (2R)-2-(methoxymethyl)pyrrolidin-1-yl, 2-(methylsulfanylmethyl)pyrrolidin-1-yl, 2-vinyl-pyrrolidin-1-yl, 2-(N-(2-(4-fluorosulfony-phenyl)-ethyl)-amino-carbonyl)-pyrrolidin-1-yl, 2,2-diallyl-pyrrolidin-1-yl, 2-(4-phenyl-phenyl)-pyrrolidin-1-yl, 3-(N-(3-acryloylamino-phenyl)-amino-)-3-hydroxy-pyrrolidin-1-yl, 3-(4-acryloyl-phenyl)-3-hydroxy-pyrrolidin-1-yl, 3-(3-acryloyl-phenyl)-3-hydroxy-pyrrolidin-1-yl, 3-hydroxy-3-(3-vinylsulfonylphenyl)pyrrolidin-1-yl, 3-(3-fluorosulfony-phenyl)-3-hydroxy-pyrrolidin-1-yl, 3-(4-fluorosulfony-phenyl)-3-hydroxy-pyrrolidin-1-yl, 2-(2,3,4,5,6-pentafluorophenyl)oxycarbonyl-pyrrolidin-1-yl, 2-(2,3,4,5,6-pentafluorophenyl)oxycarbonylmethyl-pyrrolidin-1-yl, morpholin-4-yl, piperidin-1-yl, 3-fluoro-piperidin-1-yl, 3,3-difluoro-piperidin-1-yl, 3-chloro-piperidin-1-yl, 3-hydroxy-piperidin-1-yl, 3-methoxy-piperidin-1-yl, 3-hydroxy-3-phenyl-piperidin-1-yl, (S)-3-hydroxy-3-phenyl-piperidin-1-yl, (R)-3-hydroxy-3-phenyl-piperidin-1-yl, 3-benzyl-3-hydroxy-piperidin-1-yl, (R)-5,5-difluoro-3-hydroxy-piperidin-1-yl, (S)-5,5-difluoro-3-hydroxy-piperidin-1-yl, 2-(4-methoxy-benzyl)-piperidin-1-yl, 2-(2-methoxy-benzyl)-piperidin-1-yl, (R)-2-(2-methoxy-benzyl)-piperidin-1-yl, (S)-2-(2-methoxy-benzyl)-piperidin-1-yl, 2-(2-chloro-benzyl)-piperidin-1-yl, 2-Benzofuran-2-yl-piperidin-1-yl, 3,4-dihydro-2H-quinolin-1-yl, 2-methyl-2,3-dihydro-indol-1-yl, 6-(N-(2-(4-acryloylamino-phenyl)-ethyl)-amino-carbonyl)-piperidin-1-yl, (R)-2-(4-methoxy-benzyl)-piperidin-1-yl, (S)-2-(4-methoxy-benzyl)-piperidin-1-yl, 2-(N-(2-(4-fluorosulfony-phenyl)-ethyl)-amino-carbonyl)-piperidin-1-yl, [4-(2-{[1-(2-Amino-6-methylamino-pyrimidin-4-yl)-piperidine-4-carbonyl]-amino}-ethyl)-phenyl]-carbamic acid tert-butyl ester, 8-oxa-3-azabicyclo[3.2.1]octan-3-yl, 6′-fluoro-4′-hydroxy-spiro[azetidine-3,2′-chromane]-1-yl, and 6-(trifluoromethyl)-2-azabicyclo[3.1.0]hexan-2-yl.

Preferred compounds according to the present invention, preferably for use in the treatment of cancer, are the following:
[1-(2-amino-6-cyclopropylamino-pyrimidin-4-yl)-pyrrolidin-3-yl]-methanol
6-(3,3-difluoro-pyrrolidin-1-yl)-N⁴-methyl-pyrimidine-2,4-diamine
N⁴-cyclopropyl-6-(3,3-difluoro-pyrrolidin-1-yl)-pyrimidine-2,4-diamine
1-(2-amino-6-cyclopropylamino-pyrimidin-4-yl)-pyrrolidine-3-carboxylic acid amide-formate
1-(2-amino-6-cyclopropylamino-pyrimidin-4-yl)-pyrrolidin-3-ol
1-(2-amino-6-methylamino-pyrimidin-4-yl)-pyrrolidin-3-ol-formate
(R)-1-(2-amino-6-methylamino-pyrimidin-4-yl)-pyrrolidin-2-yl]-methanol-formate
6-(3-methoxy-pyrrolidin-1-yl)-N⁴-methyl-pyrimidine-2,4-diamine-formate
6-(3-methoxy-pyrrolidin-1-yl)-N⁴-methyl-pyrimidine-2,4-diamine
N⁴-cyclopropyl-6-(3-methoxy-pyrrolidin-1-yl)-pyrimidine-2,4-diamine-formate
N⁴-cyclopropyl-6-(3-methoxy-pyrrolidin-1-yl)-pyrimidine-2,4-diamine
[(R)-1-(2-amino-6-cyclopropylamino-pyrimidin-4-yl)-pyrrolidin-2-yl]-methanol-formate
[(R)-1-(2-amino-6-cyclopropylamino-pyrimidin-4-yl)-pyrrolidin-2-yl]-methanol
[(S)-1-(2-amino-6-methylamino-pyrimidin-4-yl)-pyrrolidin-2-yl]-methanol
[(S)-1-(2-amino-6-cyclopropylamino-pyrimidin-4-yl)-pyrrolidin-2-yl]-methanol
6-(3,3-difluoro-piperidin-1-yl)-N⁴-methyl-pyrimidine-2,4-diamine
6-(3-methoxy-piperidin-1-yl)-N⁴-methyl-pyrimidine-2,4-diamine
1-(2-amino-6-methylamino-pyrimidin-4-yl)-3-benzyl-piperidin-3-ol
(R)-1-(2-amino-6-methylamino-pyrimidin-4-yl)-5,5-difluoro-piperidin-3-ol
(S)-1-(2-amino-6-methylamino-pyrimidin-4-yl)-5,5-difluoro-piperidin-3-ol
6-azetidin-1-yl-N⁴-methyl-pyrimidine-2,4-diamine
6-(3,3-difluoro-azetidin-1-yl)-N⁴-methyl-pyrimidine-2,4-diamine
1-(2-amino-6-methylamino-pyrimidin-4-yl)-azetidin-3-one
N⁴-methyl-6-(2-oxa-6-aza-spiro[3.3]hept-6-yl)-pyrimidine-2,4-diamine
6-[2-(4-fluoro-phenyl)-azetidin-1-yl]-N⁴-methyl-pyrimidine-2,4-diamine
N⁴-methyl-6-(8-oxa-3-azabicyclo[3.2.1]octan-3-yl)pyrimidine-2,4-diamine
1-[2-amino-6-(methylamino)pyrimidin-4-yl]-6′-fluoro-spiro[azetidine-3,2′-chromane]-4′-ol
(R)-1-(2-Amino-6-cyclopropylamino-pyrimidin-4-yl)-pyrrolidin-3-ol
N4-Cyclopropyl-6-(2-phenyl-pyrrolidin-1-yl)-pyrimidine-2,4-diamine
N4-Cyclopropyl-6-(2-methyl-octahydro-indol-1-yl)-pyrimidine-2,4-diamine
1-(2-Amino-6-cyclopropylamino-pyrimidin-4-yl)-3-phenyl-pyrrolidin-3-ol
N4-Cyclopropyl-6-(2-methyl-pyrrolidin-1-yl)-pyrimidine-2,4-diamine
6-(2-Benzyl-pyrrolidin-1-yl)-N4-cyclopropyl-pyrimidine-2,4-diamine
N4-Cyclopropyl-6-(2,3-dihydro-indol-1-yl)-pyrimidine-2,4-diamine
6-[2-(2-Chloro-benzyl)-pyrrolidin-1-yl]-N4-cyclopropyl-pyrimidine-2,4-diamine
(S)-1-(2-Amino-6-cyclopropylamino-pyrimidin-4-yl)-3-phenyl-pyrrolidin-3-ol
(R)-1-(2-Amino-6-cyclopropylamino-pyrimidin-4-yl)-3-phenyl-pyrrolidin-3-ol
N4-Cyclopropyl-6-(2,3-dimethyl-pyrrolidin-1-yl)-pyrimidine-2,4-diamine
1-(2-Amino-6-cyclopropylamino-pyrimidin-4-yl)-3-ethyl-pyrrolidin-3-ol
N4-Cyclopropyl-6-(2-methyl-3-phenyl-pyrrolidin-1-yl)-pyrimidine-2,4-diamine
1-(2-Amino-6-cyclopropylamino-pyrimidin-4-yl)-3-trifluoromethyl-pyrrolidin-3-ol
1-(2-Amino-6-cyclopropylamino-pyrimidin-4-yl)-3-methyl-pyrrolidin-3-ol
(S)-1-(2-Amino-6-cyclopropylamino-pyrimidin-4-yl)-pyrrolidin-3-ol
(3S,5R)-1-(2-Amino-6-cyclopropylamino-pyrimidin-4-yl)-5-methyl-pyrrolidin-3-ol
(3R,5S)-1-(2-Amino-6-cyclopropylamino-pyrimidin-4-yl)-5-methyl-pyrrolidin-3-ol
6-[(R)-2-(2-Chloro-benzyl)-pyrrolidin-1-yl]-N4-cyclopropyl-pyrimidine-2,4-diamine
6-[(S)-2-(2-Chloro-benzyl)-pyrrolidin-1-yl]-N4-cyclopropyl-pyrimidine-2,4-diamine
(S)-1-(2-Amino-6-cyclopropylamino-pyrimidin-4-yl)-3-trifluoromethyl-pyrrolidin-3-ol
(R)-1-(2-Amino-6-cyclopropylamino-pyrimidin-4-yl)-3-trifluoromethyl-pyrrolidin-3-ol
N4-Cyclopropyl-6-(3,4-dihydro-2H-quinolin-1-yl)-pyrimidine-2,4-diamine
(R)-1-(2-Amino-6-cyclopropylamino-pyrimidin-4-yl)-3-methyl-pyrrolidin-3-ol
(S)-1-(2-Amino-6-cyclopropylamino-pyrimidin-4-yl)-3-methyl-pyrrolidin-3-ol
N4-Cyclopropyl-6-((2S,3R)-2-methyl-3-phenyl-pyrrolidin-1-yl)-pyrimidine-2,4-diamine
N4-Cyclopropyl-6-((2S,3S)-2-methyl-3-phenyl-pyrrolidin-1-yl)-pyrimidine-2,4-diamine
N4-Cyclopropyl-6-((2R,3S)-2-methyl-3-phenyl-pyrrolidin-1-yl)-pyrimidine-2,4-diamine
N4-Cyclopropyl-6-((2R,3R)-2-methyl-3-phenyl-pyrrolidin-1-yl)-pyrimidine-2,4-diamine
N4-Cyclopropyl-6-(2-methyl-2,3-dihydro-indol-1-yl)-pyrimidine-2,4-diamine
(R)-1-(2-Amino-6-cyclopropylamino-pyrimidin-4-yl)-pyrrolidine-3-carboxylic acid amide
(R)-1-(2-Amino-6-methylamino-pyrimidin-4-yl)-pyrrolidin-3-ol
(S)-1-(2-Amino-6-cyclopropylamino-pyrimidin-4-yl)-pyrrolidine-3-carboxylic acid amide
(S)-1-(2-Amino-6-methylamino-pyrimidin-4-yl)-pyrrolidin-3-ol 4-(3,3-Difluoro-pyrrolidin-1-yl)-5,6,7,8-tetrahydro-pyrido[2,3-d]pyrimidin-2-ylamine
[(R)-1-(2-Amino-6-methylamino-pyrimidin-4-yl)-pyrrolidin-3-yl]-methanol
[(S)-1-(2-Amino-6-methylamino-pyrimidin-4-yl)-pyrrolidin-3-yl]-methanol
[(R)-1-(2-Amino-6-cyclopropylamino-pyrimidin-4-yl)-pyrrolidin-3-yl]-methanol
[(S)-1-(2-Amino-6-cyclopropylamino-pyrimidin-4-yl)-pyrrolidin-3-yl]-methanol
6-(3,3-Difluoro-pyrrolidin-1-yl)-N4-ethyl-pyrimidine-2,4-diamine
6-(3,3-Difluoro-pyrrolidin-1-yl)-N4-isopropyl-pyrimidine-2,4-diamine
(R)-1-(2-Amino-5,6,7,8-tetrahydro-pyrido[2,3-d]pyrimidin-4-yl)-pyrrolidin-3-ol
N4-Cyclopropylmethyl-6-(3,3-difluoro-pyrrolidin-1-yl)-pyrimidine-2,4-diamine
6-(3,3-Difluoro-pyrrolidin-1-yl)-N4-propyl-pyrimidine-2,4-diamine
(S)-1-(2-Amino-5,6,7,8-tetrahydro-pyrido[2,3-d]pyrimidin-4-yl)-pyrrolidin-3-ol-formiate
(S)-1-(2-Amino-5,6,7,8-tetrahydro-pyrido[2,3-d]pyrimidin-4-yl)-pyrrolidin-3-ol
4-(3,3-Difluoro-pyrrolidin-1-yl)-5,6,7,8-tetrahydro-pyrido[2,3-d]pyrimidine
(R)-1-(2-Amino-5,6,7,8-tetrahydro-pyrido[2,3-d]pyrimidin-4-yl)-pyrrolidine-3-carboxylic acid amide-trifluoroacetate
(R)-1-(2-Amino-5,6,7,8-tetrahydro-pyrido[2,3-d]pyrimidin-4-yl)-pyrrolidine-3-carboxylic acid amide
N4-Cyclopentylmethyl-6-(3,3-difluoro-pyrrolidin-1-yl)-pyrimidine-2,4-diamine 6-(3,3-Difluoro-pyrrolidin-1-yl)-N4-[2-(2-methoxy-phenyl)-ethyl]-pyrimidine-2,4-diamine
(S)-1-(2-Amino-5,6,7,8-tetrahydro-pyrido[2,3-d]pyrimidin-4-yl)-pyrrolidine-3-carboxylic acid amide-formiate
(S)-1-(2-Amino-5,6,7,8-tetrahydro-pyrido[2,3-d]pyrimidin-4-yl)-pyrrolidine-3-carboxylic acid amide
1-(2-Amino-6-methylamino-pyrimidin-4-yl)-3-phenyl-piperidin-3-ol
1-(2-Amino-6-cyclopropylamino-pyrimidin-4-yl)-pyrrolidine-2-carboxylic acid
6-(2-Benzofuran-2-yl-piperidin-1-yl)-N4-methyl-pyrimidine-2,4-diamine
6-[2-(2-Methoxy-benzyl)-pyrrolidin-1-yl]-N4-methyl-pyrimidine-2,4-diamine
N4-Methyl-6-[2-(1-methyl-1-phenyl-ethyl)-pyrrolidin-1-yl]-pyrimidine-2,4-diamine
6-[2-(4-Chloro-benzyl)-pyrrolidin-1-yl]-N4-methyl-pyrimidine-2,4-diamine
6-[2-(3-Chloro-benzyl)-pyrrolidin-1-yl]-N4-methyl-pyrimidine-2,4-diamine
(R)-1-(2-Amino-6-cyclopropylamino-pyrimidin-4-yl)-4-hydroxy-pyrrolidin-2-one
[1-(2-Amino-6-cyclopropylamino-pyrimidin-4-yl)-pyrrolidin-2-yl]-acetic acid butyl ester
1-(2-Amino-6-cyclopropylamino-pyrimidin-4-yl)-pyrrolidine-2-carboxylic acid
2-chloro-benzylamide
N4-Methyl-6-[2-(2-methyl-benzyl)-pyrrolidin-1-yl]-pyrimidine-2,4-diamine
(R)-1-(2-Amino-6-methylamino-pyrimidin-4-yl)-3-phenyl-piperidin-3-ol
(S)-1-(2-Amino-6-methylamino-pyrimidin-4-yl)-3-phenyl-piperidin-3-ol
6-[(S)-2-(2-Methoxy-benzyl)-pyrrolidin-1-yl]-N4-methyl-pyrimidine-2,4-diamine
6-[(R)-2-(2-Methoxy-benzyl)-pyrrolidin-1-yl]-N4-methyl-pyrimidine-2,4-diamine
1-(2-Amino-6-cyclopropylamino-pyrimidin-4-yl)-pyrrolidin-2-one
6-[2-(4-Methoxy-benzyl)-piperidin-1-yl]-N4-methyl-pyrimidine-2,4-diamine
1-(2-Amino-6-methylamino-pyrimidin-4-yl)-4-phenyl-pyrrolidin-2-one
[1-(2-Amino-6-cyclopropylamino-pyrimidin-4-yl)-pyrrolidin-2-yl]-acetic acid
6-[2-(2-Chloro-benzyl)-pyrrolidin-1-yl]-N4-methyl-pyrimidine-2,4-diamine
(S)-1-(2-Amino-6-methylamino-pyrimidin-4-yl)-5-hydroxymethyl-pyrrolidin-2-one
6-[2-(2-Methoxy-benzyl)-piperidin-1-yl]-N4-methyl-pyrimidine-2,4-diamine
6-(2-Isopropyl-pyrrolidin-1-yl)-N4-methyl-pyrimidine-2,4-diamine-formate
6-(2-Isopropyl-pyrrolidin-1-yl)-N4-methyl-pyrimidine-2,4-diamine
6-(2-Cyclopentyl-pyrrolidin-1-yl)-N4-methyl-pyrimidine-2,4-diamine
1-(2-Amino-6-methylamino-pyrimidin-4-yl)-3-phenyl-pyrrolidin-2-one
1-(2-Amino-6-cyclopropylamino-pyrimidin-4-yl)-5-(4-chloro-phenyl)-pyrrolidin-2-one
6-[2-(2-Chloro-benzyl)-piperidin-1-yl]-N4-methyl-pyrimidine-2,4-diamine
6-[(R)-2-(2-Methoxy-benzyl)-piperidin-1-yl]-N4-methyl-pyrimidine-2,4-diamine
6-[(S)-2-(2-Methoxy-benzyl)-piperidin-1-yl]-N4-methyl-pyrimidine-2,4-diamine
4-(2-{[1-(2-Amino-6-methylamino-pyrimidin-4-yl)-azetidine-3-carbonyl]-amino}-ethyl)-benzenesulfonyl fluoride
1-(2-Amino-6-cyclopropylamino-pyrimidin-4-yl)-pyrrolidine-2-carboxylic acid phenylamide
[4-(2-{[1-(2-Amino-6-methylamino-pyrimidin-4-yl)-piperidine-4-carbonyl]-amino}-ethyl)-phenyl]-carbamic acid tert-butyl ester
N4-Methyl-6-(2-thiophen-3-yl-pyrrolidin-1-yl)-pyrimidine-2,4-diamine
N4-Methyl-6-[2-(3-methyl-benzyl)-pyrrolidin-1-yl]-pyrimidine-2,4-diamine
1-(2-Amino-6-methylamino-pyrimidin-4-yl)-piperidine-4-carboxylic acid [2-(4-acryloylamino-phenyl)-ethyl]-amide
1-(2-Amino-6-methylamino-pyrimidin-4-yl)-5-benzyl-pyrrolidin-2-one
1-(2-Amino-6-methylamino-pyrimidin-4-yl)-4-benzyl-pyrrolidin-2-one
6-[(R)-2-(4-Methoxy-benzyl)-piperidin-1-yl]-N4-methyl-pyrimidine-2,4-diamine
6-[(S)-2-(4-Methoxy-benzyl)-piperidin-1-yl]-N4-methyl-pyrimidine-2,4-diamine
1-(2-Amino-6-methylamino-pyrimidin-4-yl)-azetidine-3-carboxylic acid [2-(4-acryloylamino-phenyl)-ethyl]-amide
4-(2-{[1-(2-Amino-6-cyclopropylamino-pyrimidin-4-yl)-pyrrolidine-2-carbonyl]-amino}-ethyl)-benzenesulfonyl fluoride
4-(2-{[1-(2-Amino-6-methylamino-pyrimidin-4-yl)-piperidine-4-carbonyl]-amino}-ethyl)-benzenesulfonyl fluoride
N4-methyl-6-[2-(2-phenylethyl)pyrrolidin-1-yl]pyrimidine-2,4-diamine
6-[(2S)-2-(methoxymethyl)pyrrolidin-1-yl]-N4-methyl-pyrimidine-2,4-diamine
N4-methyl-6-[2-(methylsulfanylmethyl)pyrrolidin-1-yl]pyrimidine-2,4-diamine
6-[(2R)-2-(methoxymethyl)pyrrolidin-1-yl]-N4-methyl-pyrimidine-2,4-diamine
[(2S)-1-[2-amino-6-(methylamino)pyrimidin-4-yl]pyrrolidin-2-yl]-diphenyl-methanol
[(2R)-1-[2-amino-6-(methylamino)pyrimidin-4-yl]pyrrolidin-2-yl]-diphenyl-methanol
6-(2-isobutylpyrrolidin-1-yl)-N4-methyl-pyrimidine-2,4-diamine
6-[(2S)-2-(bromomethyl)pyrrolidin-1-yl]-N4-methyl-pyrimidine-2,4-diamine
1-[2-amino-6-(methylamino)pyrimidin-4-yl]pyrrolidine-2-carbaldehyde
6-(2,2-diallylpyrrolidin-1-yl)-N4-methyl-pyrimidine-2,4-diamine
N4-methyl-6-[2-(4-phenylphenyl)pyrrolidin-1-yl]pyrimidine-2,4-diamine
N-[3-[1-[2-amino-6-(methylamino)pyrimidin-4-yl]-3-hydroxy-pyrrolidin-3-yl]phenyl]prop-2-enamide
1-[4-[1-[2-amino-6-(methylamino)pyrimidin-4-yl]-3-hydroxy-pyrrolidin-3-yl]phenyl]prop-2-en-1-one
3-[1-[2-amino-6-(methylamino)pyrimidin-4-yl]-3-hydroxy-pyrrolidin-3-yl]benzaldehyde
1-[2-amino-6-(methylamino)pyrimidin-4-yl]-3-(3-vinylsulfonylphenyl)pyrrolidin-3-ol
3-[1-[2-amino-6-(methylamino)pyrimidin-4-yl]-3-hydroxy-pyrrolidin-3-yl]benzenesulfonyl fluoride
4-[1-[2-amino-6-(methylamino)pyrimidin-4-yl]-3-hydroxy-pyrrolidin-3-yl]benzenesulfonyl fluoride
1-[3-[1-[2-amino-6-(methylamino)pyrimidin-4-yl]-3-hydroxy-pyrrolidin-3-yl]phenyl]ethanone
6-[2-(4-fluorophenyl)azetidin-1-yl]-N4-methyl-pyrimidine-2,4-diamine
6-(3-benzyloxyazetidin-1-yl)-N4-methyl-pyrimidine-2,4-diamine
(2,3,4,5,6-pentafluorophenyl) 1-[2-amino-6-(methylamino)pyrimidin-4-yl]pyrrolidine-2-carboxylate
(2,3,4,5,6-pentafluorophenyl) 2-[1-[2-amino-6-(methylamino)pyrimidin-4-yl]pyrrolidin-2-yl]acetate
N4-methyl-6-[6-(trifluoromethyl)-2-azabicyclo[3.1.0]hexan-2-yl]pyrimidine-2,4-diamine,
4-(3,3-Difluoro-pyrrolidin-1-yl)-5,6,7,8-tetrahydro-pyrido[2,3-d]pyrimidin-2-ylamine,
6-(3,3-Difluoro-pyrrolidin-1-yl)-N4-(2-p-tolyl-ethyl)-pyrimidine-2,4-diamine-formiate
6-(3,3-Difluoro-pyrrolidin-1-yl)-N4-(2-p-tolyl-ethyl)-pyrimidine-2,4-diamine or pharmaceutically acceptable salts, solvates, stereoisomers or tautomers thereof.

The present invention also provides the following compounds, which were known as such before, for use as a medicament, preferably for use in the treatment of cancer:
1-(2-amino-6-methylamino-pyrimidin-4-yl)-piperidin-3-ol, N⁴-methyl-6-(8-oxa-3-azabicyclo[3.2.1]octan-3-yl)pyrimidine-2,4-diamine,
N⁴-methyl-6-piperidin-1-yl-pyrimidine-2,4-diamine,
N⁴-methyl-6-pyrrolidin-1-yl-pyrimidine-2,4-diamine,
N⁴-methyl-6-morpholin-4-yl-pyrimidine-2,4-diamine,
1-(2-amino-6-methylamino-pyrimidin-4-yl)-pyrrolidin-3-ol,
[1-(2-amino-6-methylamino-pyrimidin-4-yl)-pyrrolidin-3-yl]-methanol, as well as pharmaceutical formulations comprising one or more of the afore-mentioned compounds, preferably for use in the treatment of cancer.

### Method of Preparation

The compounds of the present invention can be prepared according to the procedures of the following schemes and examples, using appropriate materials, and as further exemplified by the specific Examples described further below. They may also be prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions which are known and suitable for the said reactions. Use can also be made of variants which are known per se, but are not mentioned here in greater detail.

Likewise, the starting materials for the preparation of compounds of the present invention can be prepared by methods as described in the examples or by methods known per se, as described in the literature of synthetic organic chemistry and known to the skilled person, or can be obtained commercially. The starting materials for the processes claimed and/or utilized may, if desired, also be formed in situ by not isolating them from the reaction mixture, but instead immediately converting them further into the compounds of the invention or intermediate compounds. On the other hand, in general it is possible to carry out the reaction stepwise.

Preferably, the reaction of the compounds is carried out in the presence of a suitable solvent and base, which is preferably inert under the respective reaction conditions.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds according to the present invention can be prepared according to standard procedures in the art, such as be the following General Method 1:

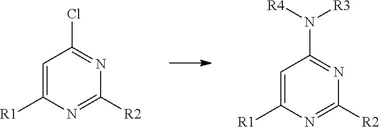

One equivalent of a pyrimidine compound comprising a suitable leaving group, typically a chloro group, or bromo, iodo, mesylate, or tosylate, is dissolved in an appropriate solvent, for instance dioxane, and a sufficient amount of cyclic amine (NR3R4) is added, typically equimolar or an excess of amine. The reaction is optionally carried out in the presence of a suitable amount of base, for instance N-ethyldiisopropylamine. The sealed flask is either heated in the microwave or under classical conditions up to 200° C. until no further conversion can be detected. At room temperature the solvent is removed in vacuum and the residue purified by chromatography.

The starting compounds are readily available or may be synthesized using techniques well known in the art.

Introducing the other substituents on the pyrimidine moiety, as far as necessary, can equally accomplished by methods well known in the art. For instance, pyrimidine compounds wherein R1 is an amino group (NR5R6) are readily obtainable by a method analogous to General Method 1, and involves reacting 4-chloropyrimidin-2-amine, for instance (optionally further substituted) and an appropriate amount (equimolar or excess) of the desired amine, such as cyclopropylamine (R5=cyclopropyl, R6=H) or methylamine (R5=methyl, R6=H), optionally in the presence of N-ethyldiisopropylamine or another suitable base, in a suitable solvent, such as ethanol or butanol, stirring at a suitable temperature, in case of ethanol for instance 85° C., in case of butanol for instance 95° C., until the reaction is complete. The reaction mixture is then worked up in a suitable manner, for instance by cooling the reaction mixture, concentration by evaporation and chromatography. In an alternative method of preparation, the amine and the 4-chloropyrimidine-2-amine can be reacted in the presence of trimethylamine (e.g. 2 eq) in butanol or isopropanol, heated in a sealed tube at 95° C. for at least 12 hours, concentration and purification. In light of various amino-pyrimidine compounds in general having been known for decades, preparation of such starting compounds is well known in the art.

By way of a further example, if one of R5 or R6 is an arylalky, for instance, the following general procedure can be followed: A suitable 2-amino-4-chloro-pyrimidine (optionally further substituted) is reacted with an equimolar amount of the arylalkylamine (or arylalkyl(alkyl) amine, such as methyl-(1-naphthalen-2-yl)ethyl)-amine), in the presence of a slight excess of cesium carbonate in a 1:1 mixture of dioxane and water in a sealed container under stirring at 210° C. for e.g. 20 minutes in a microwave reactor. Workup generally involves concentration and purification.

Similarly, the amino group at R2 can be introduced under suitable conditions starting from the corresponding 2-chloropyrimidine (optionally further substituted), for instance starting from a solution of the appropriate 2-chloropyrimidine (1 eq) in ammonium hydroxide (25% aq), heating in the microwave until the reaction is complete and work-up by evaporation of solvent and drying of the product under vacuum (General Method 4). In an alternative approach, the 2-aminopyrimidine compound can be prepared from a commercially available sulfone (CH₃—S(O)₂—→RHN—) by reaction with a suitable amine in a suitable solvent such as ethyl acetate and THF from about 0° C. to room temperature.

4-Chloro-5,6,7,8-tetrahydro-pyrido[2,3-d]pyrimidin-2-ylamine, which is a suitable starting compound for the 5,6,7,8-tetrahydro-pyrido[2,3-d]pyrimidin-2-ylaminederivatives according to the present invention (Formula II), can be prepared as described for Compound 75 below.

For examples of methods of preparing useful starting compounds and intermediates as well as methods of modifying the various substituents, it is referred to WO 2014/084778, by way of example, which is incorporated by reference in its entirety.

The general scheme depicted above, General Method 1, is exemplified in the following:

### [(R)-1-(2-Amino-6-methylamino-pyrimidin-4-yl)-pyrrolidin-3-yl]-methanol (Example 1)

6-Chloro-N4-methyl-pyrimidine-2,4-diamine (200 mg; 1.3 mmol) was dissolved in Ethanol (10 ml) and N-Ethyldiisopropylamine (0.3 ml) was added. To this solution (R)-1-Pyrrolidin-3-yl-methanol hydrochloride (200 mg; 1.5 mmol) was given and the mixture was kept for 2 h at 150° C. in the microwave. For work up the mixture was evaporated to dryness and purified by chromatography to give [(R)-1-(2-Amino-6-methylamino-pyrimidin-4-yl)-pyrrolidin-3-yl]-methanol (37 mg) as beige crystals.

### N⁴-cyclopropyl-6-(3,3-difluoropyrrolidin-1-yl)pyrimidine-2,4-diamine (Example 4)

6-Chloro-N⁴-cyclopropyl-pyrimidine-2,4-diamine (100.00 mg; 0.54 mmol) is dissolved in 1,4-dioxane (5.00 ml) and N-ethyldiisopropylamine (0.20 ml). 3,3-Difluoro-pyrrolidine hydrochloride (95.00 mg; 0.66 mmol) is added and the mixture is microwaved for 2 h at 150° C. For work up the mixture is evaporated and purified by HPLC giving 82 mg of the product as white crystals.

### N⁴-methyl-6-(8-oxa-3-azabicyclo[3.2.1]octan-3-yl)pyrimidine-2,4-diamine (Example 27)

6-Chloro-N⁴-methyl-pyrimidine-2,4-diamine (20.00 mg; 0.13 mmol) is dissolved in dimethyl sulfoxide (5.00 ml) and N-ethyldiisopropylamine (70,00 μl). 8-Oxa-3-aza-bicyclo[3.2.1]octane (21.00 mg; 0.14 mmol) is added and the mixture is heated for 2 h at 150° C. in a closed vial. For work up the mixture is lyophilized and pyrified by HPLC giving 3.5 mg of the product as yellowish foam.

### (R)-1-(2-Amino-5,6,7,8-tetrahydro-pyrido[2,3-d]pyrimidin-4-yl)-pyrrolidine-3-carboxylic acid amide-trifluoroacetate (Compound 75)

To a solution of 3-Carboxylic acid-2-piperidinone ethyl ester (2 g; 12 mmol) in DCM was added Triethyloxonium-tetrafluoroborat (2.7 g; 14 mmol) in DCM. The solution was stirred for 16 hrs at rt. In-process control via TLC Si60: eluent Heptane/Ethyl acetate=2:1 and DCM/MeOH=10:1; colored with KMnO₄—Solution.

Water (5 ml) was added to the reaction mixture and the organic layer was extracted with small amounts of an aqueous NaHCO₃-solution, dried with Na₂SO₄ and evaporated to dryness giving 2.3 g of the crude product which was used in the next step without further purification.

To a solution of 2-Ethoxy-3,4,5,6-tetrahydro-pyridine-3-carboxylic acid ethyl ester (2.3 g; 11.5 mmol) in Ethanol (30 ml) was added Guanidinium chloride (1.1 g; 11.5 mmol) and Sodium ethoxide solution (21% in ethanol; 10.8 ml; 28.9 mmol). The reaction was refluxed for 16 hrs and then evaporated to dryness giving 4.4 g of a brown oil with some inorganic impurity (product content 43%). This mixture was used in the next step without further purification.

To 2-Amino-5,6,7,8-tetrahydro-3H-pyrido[2,3-d]pyrimidin-4-one (2.5 g; 10 mmol) was added Phosphoryl chloride (3.5 ml; 38.8 mmol) carefully under ice cooling (exothermic reaction). The reaction was stirred for 16 hrs at 100° C. To the reaction mixture was added carefully a small amount of water, basified with NaOH 45% and extracted 3× with a great amount of Ethyl acetate. The combined organic layers were dried with Na₂SO₄ and evaporated to dryness giving 297 mg of the required product.

To a solution of 4-Chloro-5,6,7,8-tetrahydro-pyrido[2,3-d]pyrimidin-2-ylamine (76 mg; 0.4 mmol) in 1-Butanol (4 ml) in a microwave vial was added (R)-Pyrrolidine-3-carboxylic acid amide hydrochloride (73 mg; 0.5 mmol) and Triethylamine (0.2 ml; 1.5 mmol). The reaction was stirred for 3 days at 150° C. The reaction was evaporated to dryness and the residue was purified by prep. HPLC giving 25 mg of the desired product as white solid.

### 6-(3,3-Difluoro-pyrrolidin-1-yl)-N4-[2-(2-methoxy-phenyl)-ethyl]-pyrimidine-2,4-diamine (Compound 77)

2-Amino-4,6-Dichloropyrimidine (100 mg; 0.6 mmol), 2-(2-methoxyphenyl)ethan amine (100 mg; 0.7 mmol) and N-Ethyl diisopropyl amine (0.1 ml; 0.7 mmol) were dissolved in acetonitrile (4 ml). The reaction was refluxed for 3 days. The resulting yellow solution was evaporated under vacuo and extracted with ethyl acetate/water. The organic layer was dried over sodium sulfate, filtered and evaporated to dryness giving 130 mg of a yellow oil which was used in the next step without further purification.

6-chloro-N4-[2-(2-methoxyphenyl)ethyl]pyrimidine-2,4-diamine (130 mg; 0.4 mmol) and 3,3-Difluoropyrrolidine hydrochloride (63 mg; 0.4 mmol) were dissolved in 1,4-dioxane (3 ml). N-Ethyl diisopropyl amine (0.2 ml) was added and the reaction mixture was heated to 170° C. for 4 h in the microwave. The reaction mixture was evaporated and the residue was extracted with ethyl acetate/water. The organic layer was dried over sodium sulfate, filtered and evaporated to dryness and the residue was purified two times by HPLC. The combined product fractions were extracted with ethyl acetate and the organic layer was dried over sodium sulfate, filtered and evaporated to dryness giving 15 mg after the second run of the product as yellow oil.

### 1-(2-Amino-6-cyclopropylamino-pyrimidin-4-yl)-pyrrolidine-2-carboxylic acid (Compound 80)

To a solution of 6-Chloro-N4-cyclopropyl-2,4-pyrimidinediamine (60 mg; 0.3 mmol) in 1-Butanol (3.0 ml) was added DL-PROLINE (49 mg; 0.4 mmol) in a microwave vial and 1,8-Diazabicyclo[5.4.0]undec-7-ene (0.2 ml; 1.1 mmol). The reaction was stirred for 16 hrs at 150° C. The reaction was evaporated to dryness and the residue purified by chromatography giving 41 mg of the product as colorless solid.

### 1-(2-Amino-6-cyclopropylamino-pyrimidin-4-yl)-pyrrolidine-2-carboxylic acid 2-chloro-benzylamide (Compound 88)

To a solution of 1-(2-Amino-6-cyclopropylamino-pyrimidin-4-yl)-pyrrolidine-2-carboxylic acid (300 mg; 0.4 mmol) in N,N-Dimethylformamide (10 ml) was added 2-Chlorobenzylamine (0.1 ml; 0.4 mmol) and N-(3-Dimethylaminopropyl)-N′-ethyl carbodiimide hydrochloride (94 mg; 0.5 mmol). The reaction was stirred for 3 days at rt. The reaction was diluted with ethyl acetate and extracted 3× with water, dried over Na2SO4 and evaporated to dryness. The residue was purified by prep. HPLC giving 36 mg of the product as colorless solid.

### 6-[2-(4-Methoxy-benzyl)-piperidin-1-yl]-N4-methyl-pyrimidine-2,4-diamine (Compound 95)

6-Chloro-N4-methyl-pyrimidine-2,4-diamine (200 mg; 1.3 mmol) was dissolved in 1-Butanol (5 ml) and N-Ethyldiisopropyl amine (0.85 ml) was added. After the addition of 2-(4-Methoxy-benzyl)-piperidine hydrochloride (300 mg; 1.2 mmol) the mixture was stirred 72 h at 180° C. For work up the reaction mixture was evaporated to dryness and purified by präp-HPLC giving 30 mg of the desired product as beige solid.

### 6-[2-(2-Methoxy-benzyl)-piperidin-1-yl]-N4-methyl-pyrimidine-2,4-diamine (Compound 100) and enantiomers (Compounds 106 & 107)

6-Chloro-N4-methyl-pyrimidine-2,4-diamine (200 mg; 1.3 mmol) was dissolved in 1-Butanol (5 ml) and N-Ethyl diisopropyl amine (0.6 ml). 2-(2-Methoxy-benzyl)-piperidine (310 mg 1.5 mmol) was added and the mixture was stirred for 72 h at 180° C. in a closed vessel. The reaction was evaporated and the residue purified by HPLC giving 56 mg of the product as beige crystals.

The enantiomers of the racemic compound were isolated via SFC chromatography using the Lux-Cellulose-2 column and as eluent: COO₂/Methanol+0.5% DEA=65:35 with a flow of 5 ml/min. 50 mg of racemate were dissolved in 1.2 ml Methanol/Dioxane=1:1 and portions of 85 μl were infected. 9 mg of each enantiomer were obtained. The absolute configuration of the asymmetric center was assigned arbitrarily.

### 1-(2-Amino-6-methylamino-pyrimidin-4-yl)-3-phenyl-pyrrolidin-2-one (Compound 103)

6-Chloro-N4-methyl-2,4-pyrimidinediamine (150 mg; 0.9 mmol) was dissolved in 1,4-Dioxane (5 ml) and under nitrogen was added 3-phenyl-pyrrolidin-2-one (168 mg; 1 mmol), 4,5-Bis(diphenylphosphino)-9,9-dimethylxanthene (55 mg; 0.1 mmol), potassium (Ill) phosphate (402 mg; 1.9 mmol) and Tris-(dibenzylidenaceton)-dipalladium (0) (87 mg; 0.1 mmol). The reaction was stirred for 4 hrs at 120° C. in the microwave. The reactions were filtrated and evaporated to dryness. The residue was purified by prep. HPLC giving 16 mg of the desired product as light brown solid.

### 4-(2-{[1-(2-Amino-6-methylamino-pyrimidin-4-yl)-azetidine-3-carbonyl]-amino}-ethyl)-benzenesulfonyl fluoride (Compound 108)

6-Chloro-N4-methyl-pyrimidine-2,4-diamine (500 mg; 3 mmol) was dissolved in tert. butyl alcohol (15 ml) and 1,8-Diazabicyclo[5.4.0]undec-7-ene (1 ml) was added. After the addition of Azetidine-3-carboxylic acid (400 mg) the mixture was stirred at 160° C. for 48 h. Then the mixture was evaporated in vacuum and the residue (1 g, brown oil) was used in the next step without further purification

1-(2-Amino-6-methylamino-pyrimidin-4-yl)-azetidine-3-carboxylic acid (500 mg; 1 mmol) prepared as described before was dissolved in N,N-Dimethyl formamide (5 ml) in a microwave vessel. N-Ethyl diisopropyl amine (0.6 ml), N-(3-Dimethylaminopropyl)-N′-ethyl carbodiimide hydrochloride (325 mg; 1.7 mmol) and 1-Hydroxybenzotriazole hydrate (235 mg; 1.7 mmol) were added and the mixture was stirred at room temperature for 30 min. Then 4-(2-aminoethyl)benzenesulfonyl fluoride (320 mg; 1.3 mmol) was added and after closing the vessel the mixture was stirred for 2 h at 80° C. giving a yellow solution. The reaction mixture was evaporated to dryness and the residue was dissolved in ethyl acetate/water (pH>7, with 1N NaOH). The organic layer was dried over sodium sulfate, filtered and evaporated to dryness. The residue was purified by chromatography giving 4-(2-{[1-(2-Amino-6-methylamino-pyrimidin-4-yl)-azetidine-3-carbonyl]-amino}-ethyl)-benzenesulfonyl fluoride as beige solid (6 mg).

### N4-Methyl-6-[2-(1-methyl-1-phenyl-ethyl)-pyrrolidin-1-yl]-pyrimidine-2,4-diamine (Compound 83)

6-Chloro-N4-methyl-pyrimidine-2,4-diamine (50 mg; 0.3 mmol) was dissolved in 1-Butanol (4 ml) and N-Ethyldiisopropylamine (0.13 ml). 2-(1-Methyl-1-phenyl-ethyl)-pyrrolidine (65 mg; 0.35 mmol) was added and the reaction was stirred at 160° C. for 6 days turning into a yellow solution. The reaction mixture was evaporated under vacuo. The residue was extracted with ethyl acetate/water. The organic layer was dried over sodium sulfate, filtered and evaporated under vacuum. The residue was purified by chromatography. All fractions with product mass were combined and basified with 1N NaOH. The resulting precipitate was filtered by suction giving 14 mg of the product as white solid.

### N4-Methyl-6-[2-(2-methyl-benzyl)-pyrrolidin-1-yl]-pyrimidine-2,4-diamine (Compound 89)

6-Chloro-N4-methyl-pyrimidine-2,4-diamine (50 mg; 0.3 mmol) was dissolved in 1-Butanol (4 ml) and N-Ethyldiisopropylamine (0.13 ml). To this solution 2-(o-tolylmethyl)pyrrolidine (61 mg; 0.3 mmol) was added and the reaction was stirred at 150° C. for 3 days turning into yellow. The reaction mixture was evaporated to dryness under vacuum and the residue was extracted with ethyl acetate/water. The organic layer was dried over sodium sulfate, filtered and evaporated under vacuum. The residue was purified by chromatography.

All fractions with product mass were combined, basified with 1N NaOH and extracted with ethyl acetate/water. The organic layer was dried over sodium sulfate, filtered and evaporated under vacuum giving 44 mg of the product as white solid.

### 6-[(S)-2-(2-Methoxy-benzyl)-pyrrolidin-1-yl]-N4-methyl-pyrimidine-2,4-diamine (Compound 92)

### Via Synthesis of Racemic 6-[2-(2-Methoxy-benzyl)-pyrrolidin-1-yl]-N4-methyl-pyrimidine-2,4-diamine (Compound 82)

6-Chloro-N4-methyl-pyrimidine-2,4-diamine (50 mg; 0.3 mmol) was dissolved in 1-Butanol extra pure NF (4 ml) and N-Ethyldiisopropylamine for synthesis (0.13 ml) was added followed by 2-[(2-methoxyphenyl)methyl]pyrrolidine (66 mg; 0.33 mmol). The reaction was stirred at 160° C. over night during which the color changed into a yellow solution.

The mixture was evaporated under vacuum. The residue was extracted with ethyl acetate/water. The organic layer was dried over sodium sulphate, filtered and evaporated under vacuum. The residue was suspended in diethylether and filtered by suction giving 55 mg of the product as brown solid. 50 mg of the racemic mixture (compound 82) were dissolved in 1.6 ml methanol/dioxane=1:1 and portions of 85 μl of this solution were used in chromatography with SCF yielding 23.2 mg and 23.6 mg of the enantiomers.

### Column: Lux Cellulose-2.

### Eluent: CO2/methanol+0.5% DEA=60:40

flow 5 ml/min;
wave length: 220 nm

### 6-[2-(2-Chloro-benzyl)-pyrrolidin-1-yl]-N4-methyl-pyrimidine-2,4-diamine (Compound 98)

To a solution of 6-Chloro-N4-methyl-pyrimidine-2,4-diamine (80 mg; 0.5 mmol) in 1-Butanol (3 ml) in a microwave vial was added 2-(2-chlorobenzyl)pyrrolidine hydrochloride (152 mg; 0.7 mmol) and N-Ethyldiisopropylamine (0.3 ml). The reaction was stirred for 16 hrs at 150° C. and evaporated in vacuum for work-up. The residue was purified by HPLC giving 105 mg of the product as white solid.

### 6-[(R)-2-(4-Methoxy-benzyl)-piperidin-1-yl]-N4-methyl-pyrimidine-2,4-diamine (Compound 116) & 6-[(S)-2-(4-Methoxy-benzyl)-piperidin-1-yl]-N4-methyl-pyrimidine-2,4-diamine (Compound 117)

The synthesis of 6-[2-(4-Methoxy-benzyl)-piperidin-1-yl]-N4-methyl-pyrimidine-2,4-diamine (racemic) (Compound 95) was carried out as described above with 400 mg 6-Chloro-N4-methyl-pyrimidine-2,4-diamine and 600 mg 2-(4-Methoxy-benzyl)-piperidine hydrochloride, giving 55 mg of the product.

55 mg of the racemic mixture were dissolved in 1 ml methanol/dioxane=1:1 and portions of 30 μl of this solution were used in chromatography with SCF yielding 12.1 mg of each enantiomer.

### Column: Lux Cellulose-2.

### Eluent: CO₂/methanol+0.5% HCOOH=65:35

flow 5 ml/min;
wave length: 220 nm

### 6-[2-(2-Chloro-benzyl)-piperidin-1-yl]-N4-methyl-pyrimidine-2,4-diamine (Compound 105)

6-Chloro-N4-methyl-pyrimidine-2,4-diamine (200 mg; 1.3 mmol) was dissolved in 1-Butanol (5 ml) and N-Ethyldiisopropylamine (0.6 ml) was added together with 2-(2-Chloro-benzyl)-piperidine (250 mg; 1.2 mmol). The reaction was stirred for 72 h at 180° C. For work-up the reaction was evaporated in vacuum and purified by HPLC giving 8 mg of the product as beige solid.

### 4-(2-{[1-(2-Amino-6-methylamino-pyrimidin-4-yl)-piperidine-4-carbonyl]-amino}-ethyl)-benzenesulfonyl fluoride (Compound 120)

In a microwave vessel 1-(2-Amino-6-methylamino-pyrimidin-4-yl)-piperidine-4-carboxylic acid (200 mg; 0.8 mmol) was dissolved in N,N-Dimethylformamide (5 ml). N-Ethyldiisopropylamine (0.47 ml) and 4-(2-Amino-ethyl)-benzenesulfonyl fluoride hydrochloride (230 mg; 0.96 mmol) were added followed by 1-Propylphosphonic acid cyclic anhydride 50% in DMF (T3P) (1 g; 1.6 mmol). The closed vessel was stirred for 12 h at 60° C. giving a yellow solution. For work up the reaction mixture was evaporated under vacuum to dryness. The residue was extracted with ethyl acetate/water (pH>7, with 1N NaOH). The organic layer was dried over sodium sulfate, filtered and evaporated to dryness. The residue was purified by chromatography giving 48 mg of the product as white solid.

The remainder of the Examples and compounds of the present invention can be prepared in an analogous manner, as will be readily apparent to the skilled person.

### Pharmaceutically Acceptable Salts

Pharmaceutically acceptable salts include acid addition and base salts of the compounds according to the invention. Pharmaceutically acceptable salts, which can be derived from various organic and inorganic acids and bases by procedures known in the art. Pharmaceutically acceptable salt forms of the compounds of the Formula I are prepared by conventional methods. If the compound of the Formula I contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the compounds of the Formula I are likewise included. In the case of certain compounds of the formula I, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as formate, acetate, trifluoroacetate, tartrate, maleate, succinate, citrate, benzoate, salicylate, ascorbate and the like. Accordingly, pharmaceutically acceptable acid-addition salts of the compounds of the Formula I include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, formate, galacterate (from mucic acid), galacturonate, glucoheptanoate, gluconate, glutamate, glycero-phosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, mono-hydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction. Formate is particularly preferred.

Furthermore, the base salts of the compounds according to the invention include aluminium, ammonium, calcium, copper, iron(III), iron(II), lithium, magnesium, manganese(III), manganese(II), potassium, sodium and zinc salts, but this is not intended to represent a restriction. Salts of the compounds of the Formula I which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzyl-ethylenediamine (benzathine), dicyclohexylamine, diethanolamine, diethyl-amine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethyl-amine, trimethylamine, tripropylamine and tris(hydroxymethyl)methylamine (tromethamine), but this is not intended to represent a restriction.

The compounds of the present invention contain basic nitrogen-containing groups that may be quaternised using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.

The above-mentioned pharmaceutical salts which are preferred include formate, acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

Particular preference is given to formate.

The acid-addition salts of basic compounds of the Formula I may be prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds of the Formula I are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner.

If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, di-phosphate, disodium and trihydrochloride, but this is not intended to represent a restriction.

With regard to that stated above, it can be seen that the expression “pharmaceutically acceptable salt” in the present connection is taken to mean an active ingredient which comprises a compound of the Formula I in the form of one of its salts. A salt form may impart improved pharmacokinetic properties on the active ingredient compared with the free form of the active ingredient or any other salt form of the active ingredient used earlier. The pharmaceutically acceptable salt form of the active ingredient may even have a positive influence on the pharmacodynamics of this active ingredient with respect to its therapeutic efficacy in the body.

### Stereoisomers and Tautomers

The invention relates to all steroisomeric forms of the compounds of Formula I, such as enantiomeric or diastereoisomeric forms or mixtures thereof, including all possible mixtures of stereoisomers, as well as the pure stereoisomers, in particular (R)- and (S)-enantiomers. Stereoisomers, and enantiomers in particular, can be prepared by any method known in the art, for instance by a stereoselective route of synthesis, separation of racemic mixtures, such as by a selective crystallization or chromatographic separation. The invention also relates to the use of mixtures of the compounds of the formula I, for example mixtures of two diastereomers, for example in the ratio 1:1, 1:2, 1:3, 1:4, 1:5, 1:10, 1:100 or 1:1000.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution.

### Solvates and Crystal Forms

The compounds of the present invention may exist in solvated or unsolvated forms. The term “solvate” is used herein to describe a molecular complex comprising a compound of the invention and a stoichometric or non-stoichiometric amount of one or more pharmaceutically acceptable solvent molecules. If the solvent is water, the solvent is referred to as a hydrate. It is understood that the invention also relates to the solvates of the salts.

The compounds of the present invention may exist in different forms, and the amorphous form shall be encompassed by the present invention as well as all crystal forms (polymorphs) thereof.

### Prodrugs

Prodrugs of the compounds according to the present invention shall equally be included within the scope of the present invention. As used herein and unless otherwise indicated, the term “prodrug” means a derivative of a compound of Formula I that can hydrolyze, oxidize, or otherwise react under biological conditions (in vitro or in vivo) to provide an active compound, particularly a compound of formula I. Examples of prodrugs include, but are not limited to, derivatives and metabolites of a compound of Formula I that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers Gmfh).

### Isotope-Labelled Forms

The present invention shall also include isotope-labelled forms of the compounds described herein. An isotope-labelled form of a compound of the Formula I is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the Formula I by well-known methods include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively. A compound of the formula I, a prodrug thereof or a pharmaceutically acceptable salt of either, which contains one or more of the above-mentioned isotopes and/or other isotopes of other atoms is intended to be part of the present invention. An isotope-labelled compound of Formula I can be used in a number of beneficial ways. For example, an isotope-labelled compound of the Formula I into which, for example, a radioisotope, such as ³H or ¹⁴C, has been incorporated is suitable for medicament and/or substrate tissue distribution assays. These radioisotopes, i.e. tritium (³H) and carbon-14 (¹⁴C), are particularly preferred owing to simple preparation and excellent detectability. Incorporation of heavier isotopes, for example deuterium (²H), into a compound of the Formula I may have therapeutic advantages owing to the higher metabolic stability of this isotope-labelled compound. Higher metabolic stability translates directly into an increased in vivo half-life or lower dosages, which under most circumstances would represent a preferred embodiment of the present invention. An isotope-labelled compound of the Formula I can usually be prepared by carrying out the method of preparation, as exemplified elsewhere herein, replacing a non-isotope-labelled reactant by a readily available isotope-labelled reactant.

Deuterium (²H) can also be incorporated into a compound of the Formula I for the purpose of manipulating the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange.

Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: If deuterium is bonded to a carbon atom at a non-exchangeable position, rate differences of k_{M}/k_{D}=2-7 are typical. If this rate difference is successfully applied to a compound of the Formula I that is susceptible to oxidation, the profile of this compound in vivo can be drastically modified and may result in improved pharmacokinetic properties.

Deuterium-hydrogen exchange in a compound of Formula I can also be used to achieve a favourable modification of the metabolite spectrum of the starting compound in order to diminish or eliminate undesired toxic metabolites. For example, if a toxic metabolite is formed as a result of oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

Compounds and their Use

The present invention primarily relates to the use of the herein disclosed compounds in the treatment of cancer, respectively the herein disclosed compounds for use in the treatment of cancer. The present invention also encompassed the use of a compound according to the present invention for the manufacture of a medicament for the treatment of cancer.

In addition, the present invention provides novel compounds and therefore relates to the herein disclosed compounds as such. Furthermore, the present invention generally relates to the herein disclosed compounds as medicaments, respectively for use as medicaments.

The herein disclosed compounds act as inhibitors of the MTH1 protein. The present invention thus also concerns an inhibitor of the MTH1 protein, which is selected from the herein disclosed compounds, as well as the use of the herein disclosed compounds as inhibitors of the MTH1 protein. Preferably, the inhibitor has a half maximal inhibitory concentration (IC₅₀, MTH1 enzymatic assay), preferably as determined by the below disclosed method MTH1 enzymatic assay, of 100 nM or less, more preferably 50 nM or less, more preferably 20 nM, 10 nM, 5 nM, 1 nM or less.

In one aspect, the present invention concerns a method of treating cancer in a patient, comprising administering to the patient a therapeutically effective amount of a compound as described herein. In certain embodiments, the present invention concerns a method of treating cancerous tumors in a patient, comprising administering to the patient a therapeutically effective amount of a compound as described herein. Types of cancer that may be preferably treated are described further below. Particular embodiments of compounds that can advantageously be used are also disclosed herein and many individual examples given. Determination of a therapeutically effective amount is a matter of routine for the skilled physician.

The formation of cancerous tumors is typical of what is usually referred to as stage II cancer, which occurs when cancerous cells begin to grow into a small tumor within the organ of origin. Typically, cancer in this stage has not spread to other tissues or organs within the body. By contrast, when abnormal cells only begin clumping together and begin penetrating beneath the top layer of cells within the organ of origin, they still form stage I cancer. This stage of cancer describes cancer that is small and present only within the organ of origin A cancerous tumor is characterized as stage III cancer as the cancerous tumor grows (compared to Stage II), and begins to spread into the lymph nodes and surrounding tissues. Stage IV cancer develops when cancer cells spread from their point of origin to another organ within the body. This stage of cancer, which is also referred to as metastatic or secondary cancer, is the most advanced form of cancer.

The cancer to be treated may be any of Stage I, Stage II, Stage III and/or Stage IV cancer. For instance, the cancer to be treated may be Stage II, III and/or Stage IV cancer.

The cancer may be selected, for instance, from one or more of the following: Lung cancer, breast cancer, prostate cancer, ovarian cancer, bladder cancer, colon cancer, rectal cancer, renal cancer, pancreatic cancer, thyroid cancer, endometrial cancer, leukemia, melanoma, brain tumor, cervical cancer, esophageal cancer, esthesioneuroblastoma, Ewing Sarcoma, extracranial germ cell tumor, extrahepatic bile duct cancer, eye cancer, Fallopian tube cancer, gallbladder cancer, gastric cancer, germ cell tumor, head and neck cancer, heart cancer, hepatocellular cancer, liver cancer, lymphoma, Hodgkin lymphoma, non-Hodgkin lymphoma, islet cell cancer, Kaposi sarcoma, laryngeal cancer, lip and oral cavity cancer, Merkel cell carcinoma, mesothelioma, myeloma, nasal cavity and paranasal sinus cancer, nasopharyngeal cancer, neuroblastoma, parathyroid cancer, pharyngeal cancer, pituitary tumor, salivary gland cancer, skin cancer, testicular cancer, throat cancer, thymoma and thymic carcinoma, uterine cancer, vaginal cancer, and vulvar cancer.

Representative cancers that compounds of Formula I are useful for treating include, but are not limited to, cancer of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, brain, central nervous system, solid tumors and blood-borne tumors. Preferably, the cancer to be treated is at least one of lung cancer, breast cancer, prostate cancer, ovarian cancer, bladder cancer, colon and rectal cancer, renal cancer, pancreatic cancer, thyroid cancer, endometrial cancer, leukemia, melanoma, non-Hodgkin lymphoma, and brain tumor. For instance, the cancer to be treated is at least one of lung cancer, colon cancer, pancreatic cancer, breast cancer, prostate cancer, ovarian cancer and bladder cancer. Typically, the cancer to be treated is lung cancer.

A number of cancers arise as a result of mutations in oncogenes. Ras proteins are oncogenes that are frequently mutated in human cancers. They are encoded by three ubiquitously expressed genes: H-RAS, K-RAS and N-RAS. These proteins are GTPases (hydrolase enzymes that can bind and hydrolyze guanosine triphosphate (GTP)) that function as molecular switches regulating pathways responsible for proliferation and cell survival. Ras proteins are normally tightly regulated by guanine nucleotide exchange factors (GEF₅) promoting guanosine diphosphate (GDP) dissociation and GTP binding and GTPase-activating proteins (GAPs) that stimulate the intrinsic GTPase activity of Ras to switch off signalling. Aberrant Ras function is associated with hyper-proliferative developmental disorders and cancer. MTH1 suppression has been found to cause proliferative defects in cancer cells expressing mutant RAS. Therefore, the compounds of the present invention can be advantageously used in the treatment of cancer which is linked to ras mutation, particularly activating ras mutation. Examples include cancer of the biliary tract, endometrium, large intestine, lung, ovary, pancreas and small intestine.

The patient is a mammal, typically human and may be a human adult patient or a human paediatric patient. Most typically, the patient is a human adult patient. In accordance with the above, the patient may be a patient harboring RAS mutations, in particular K RAS mutations.

The present invention further comprises a method for preparing a medicament for treating cancer, comprising:
i. Determining a concentration at which a compound according to the invention effects 50% inhibition of MTH1 activity to be 100 nM or less, 75 nM or less, 50 nM or less, 25 nM or less, preferably 10 nM or less, more preferably 1 nM or less, and
ii. preparing a pharmaceutical composition comprising the compound.

50% inhibition of MTH1 activity, i.e. the IC₅₀ value, is preferably determined by the Measurement of MTH1 inhibition method (enzymatic assay) described further below. As per the usual terminology, “nM” stands for nmol/I and “μM” stands for μmol/l.

As mentioned before, reactive oxygen species (ROS) can lead to increased ROS tension and cause oxidative damage to DNA directly or to the dNTP (deoxynucleotide triphosphate) pool. Oxidative damage is typically involved in many types of cancer. The MTH1 protein has been found to sanitize oxidative damage in the dNTP pool. For instance, one of the principal products of oxidatively damaged DNA, 8-oxodGTP (8-Oxo-2′-deoxy-guanosine-5′-triphosphate) is converted by MTH1 to 8oxodGMP (8-Oxo-2′-deoxyguanosine-5′-monophosphate). MTH1 catalytic activity has been found to be increased in both lung tumors and surrounding tissue, and MTH1 over-expressed in many cancers. Overall, MTH1 catalytic activity has been found to be required for cancer cell survival, while MTH1 is non-essential in normal cells. In turn, depletion of MTH1 leads to cancer cell death. Therefore, inhibition of MTH1 is associated with selective cancer cell cytotoxicity. However, inhibition of MTH1 may also be beneficial in the treatment of other conditions involving cells that have suffered oxidative damage.

In a further aspect, the present invention more generally relates to a method of inhibiting MTH1 protein activity, comprising exposing MTH1 protein, respectively cells comprising or expressing MTH1 protein, preferably tumor cells, to an effective amount of at least one of the compounds according to the present invention. More preferably, the present invention relates to a method of inhibiting MTH1 protein activity, comprising exposing MTH1 protein, respectively cells overexpressing MTH1 protein, to an effective amount of at least one of the compounds as described herein.

The present invention also relates to the use of a compound according to the invention for the inhibition of the MTH1 protein.

Inhibition of MTH1 activity may be particularly beneficial in any cell that has suffered oxidative damage, which damage is associated with a shortening of the cell's life span. It is hypothesized that in those scenarios where through action of MTH1 and associated repair of DNA and its functional parts, the DNA pool of a damaged cell is sanitized, inhibition of MTH1 will be beneficial to eliminate any damaged cells. This applies, in particular, to those cells that overexpress MTH1 protein as a result of oxidative damage, i.e. in those cells where MTH1 protein expression is affected by oxidative damage.

Conditions and diseases that are associated with oxidative damage to the cells include: diabetes mellitus, arthritis, particularly rheumatoid arthritis, osteoarthritis, aortic valve stenosis, urolithiasis, neurodegenerative diseases such as Alzheimer's disease, Parkinson's disease, Huntington's disease, chronic fatigue syndrome, and cardiovascular diseases, such as hypertension, dyslipidemia, atherosclerosis, myocardial infarction, angina pectoris, heart failure. Based upon the underlying mechanism of action, it is hypothesized that MTH1 inhibition may prove useful in the treatment of these conditions and diseases, too.

Accordingly, the present invention also relates to compounds, salts, stereoisomers and solvates according to the present invention for use in the treatment of conditions involving cells having MTH1 protein activity, particularly expressing MTH1 protein. In particular, the present invention also relates to compounds, salts, steroisomers and solvates according to the present invention for use in the treatment of conditions involving cells overexpressing MTH1 protein. Overexpression shall designate a level of expression of MTH1 protein in a cell that is increased, in a statistically significant percentage, such as 7%, relative to a normal cell, typically as a result of the condition to be treated.

### Pharmaceutical Formulations

The present invention also relates to a pharmaceutical formulation, preferably for use in the treatment of cancer, comprising a compound as described herein, particularly a therapeutically effective amount of a compound according to Formula I.

The pharmaceutical formulation may comprise one or more of the compounds according to the present invention, and optionally a pharmaceutically acceptable excipient and/or adjuvant. The pharmaceutical formulation is preferably for use in the treatment of cancer, for instance, is at least one of lung cancer, breast cancer, prostate cancer, ovarian cancer, bladder cancer, colon and rectal cancer, renal cancer, pancreatic cancer, thyroid cancer, endometrial cancer, leukemia, melanoma, non-Hodgkin lymphoma, and brain tumor.

The pharmaceutical formulation can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tabletting machine, giving lumps of non-uniform shape, which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be en-capsulated in microcapsules. The formulation can also be prepared in such a way that the release is extended or retarded, such as, for example, by coating or embedding of particulate material in polymers, wax and the like.

The compounds of the formula I and pharmaceutically salts, stereoisomers and solvates thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds of the formula I and the salts, stereoisomers and solvates thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxy-ethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

### Combination Treatment

In exemplary embodiments of the present invention, administration of one or more of the compounds of the present invention, particularly in the treatment of cancer, may be simultaneous, sequential or in alternation with administration of at least one other pharmaceutically active ingredient respectively therapeutic agent (used synonymously herein).

Accordingly, the present invention also provides a pharmaceutical formulation comprising a therapeutically effective amount of a compound according to Formula I, and further comprising a therapeutically effective amount of a further pharmaceutically active ingredient, preferably for use in the treatment of cancer.

Accordingly, the invention also relates to a set (kit) consisting of separate packs of
(a) a therapeutically effective amount of a compound of Formula I and/or pharmaceutically acceptable salt, tautomer, stereoisomer and solvate thereof and
(b) an effective amount of a further pharmaceutically active ingredient,
   preferably for use in the treatment of cancer

The set may comprise suitable containers, such as boxes, individual bottles, bags or ampoules. The set may, for example, comprise separate ampoules, each containing an effective amount of a compound of the Formula I and/or pharmaceutically acceptable salts, stereoisomers and solvates thereof, and an effective amount of a further active ingredient in dissolved or lyophilised form.

The disclosed compounds of the formula I can be administered in combination with other known therapeutic agents, including anticancer agents. As used here, the term “anticancer agent” relates to any agent which is administered to a patient with cancer for the purposes of treating the cancer.

The anti-cancer treatment defined above may be applied as a monotherapy or may involve, in addition to the herein disclosed compounds of Formula I, conventional surgery or radiotherapy or medicinal therapy. Such medicinal therapy, e.g. a chemotherapy or a targeted therapy, may include one or more, but preferably one, of the following anti-tumor agents.

The further pharmaceutically active ingredient is thus preferably for the treatment of cancer and preferably selected from one or more of the following:

### Alkylating Agents

such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone;
apaziquone, fotemustine, glufosfamide, palifosfamide, pipobroman, trofosfamide, uramustine, TH-302⁴, VAL-083⁴;

### Platinum Compounds

such as carboplatin, cisplatin, eptaplatin, miriplatine hydrate, oxaliplatin, lobaplatin, nedaplatin, picoplatin, satraplatin;
lobaplatin, nedaplatin, picoplatin, satraplatin;

### DNA Altering Agents

such as amrubicin, bisantrene, decitabine, mitoxantrone, procarbazine, trabectedin, clofarabine;
amsacrine, brostallicin, pixantrone, laromustine^{1,3};

### Topoisomerase Inhibitors

such as etoposide, irinotecan, razoxane, sobuzoxane, teniposide, topotecan; amonafide, belotecan, elliptinium acetate, voreloxin;

### Microtubule Modifiers

such as cabazitaxel, docetaxel, eribulin, ixabepilone, paclitaxel, vinblastine, vincristine, vinorelbine, vindesine, vinflunine;
fosbretabulin, tesetaxel;

### Antimetabolites

such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur;
doxifluridine, elacytarabine, raltitrexed, sapacitabine, tegafur^{2,3}, trimetrexate;

### Anticancer Antibiotics

such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;

### Hormones/Antagonists

such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol;
acolbifene, danazol, deslorelin, epitiostanol, orteronel, enzalutamide^{1,3;}

### Aromatase Inhibitors

such as aminoglutethimide, anastrozole, exemestane, fadrozole, letrozole, testolactone;
formestane;

### Small Molecule Kinase Inhibitors

such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib;
afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, idelalisib^{1,3}, fedratinib¹, XL-647⁴;

### Photosensitizers

such as methoxsalen³;
porfimer sodium, talaporfin, temoporfin;

### Antibodies

such as alemtuzumab, besilesomab, brentuximab vedotin, cetuximab, denosumab, ipilimumab, ofatumumab, panitumumab, rituximab, tositumomab, trastuzumab, bevacizumab, pertuzumab^{2,3;}
catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3};

### Cytokines

such as aldesleukin, interferon alfa², interferon alfa2a³, interferon alfa2b^{2,3}; celmoleukin, tasonermin, teceleukin, oprelvekin^{1,3}, recombinant interferon beta-1a⁴;

### Drug Conjugates

such as denileukin diftitox, ibritumomab tiuxetan, iobenguane 1123, prednimustine, trastuzumab emtansine, estramustine, gemtuzumab, ozogamicin, aflibercept;
cintredekin besudotox, edotreotide, inotuzumab ozogamicin, naptumomab estafenatox, oportuzumab monatox, technetium (99mTc) arcitumomab^{1,3}, vintafolide^{1,3};

### Vaccines

such as sipuleucel³; vitespen³, emepepimut-S³, oncoVAX⁴, rindopepimut³, troVax⁴, MGN-1601⁴, MGN-1703⁴;

### Miscellaneous

alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat;
celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴, picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-1703⁴;
¹ Prop. INN (Proposed International Nonproprietary Name)
² Rec. INN (Recommended International Nonproprietary Names)

### ³ USAN (United States Adopted Name)

### ⁴ no INN.

In the alternative or in addition to the above. mentioned therapeutic agents, the further pharmaceutically active ingredient that may advantageously be used in combination with the compounds according to the present invention, preferably in the treatment of cancer, is an agent that increases reactive oxygen species in cells (thus ROS levels). This is hypothesized as increasing the efficacy of the treatment involving MTH1 inhibition as even more MTH1 would be required to compensate the damage resulting from oxidative stress. Exemplary compounds useful for this purpose include doxorubicin, azidothymidine, cisplatin, paclitaxel and docetaxel.

Particularly preferred are those pharmaceutically acceptable compounds that increase both reactive oxygen species in cells and have known anticancer efficacy, for instance cisplatin.

### EXEMPLARY EMBODIMENTS

Various examples of compounds of the present invention have been suggested, synthesized and/or assessed with regard to their MTH1 inhibitory activity, and some with regard to solubility and microsomal stability, as will be described in more detail below.

### Measurement of MTH1 Inhibition

The IC₅₀ value was determined by a MTH1 enzymatic assay. The assay comprises the principal steps of incubating a mixture of MTH1, the compound in question at different concentrations and 8-oxo-2′-deoxyguanosine-5′-triphosphate (8-oxo-dGTP) in assay buffer. Nucleotide triphosphate hydrolysis by MTH1, i.e. decomposition of 8-oxo-2′-deoxyguanosine-5′-triphosphate, produces 8-oxo-2′-deoxyguanosine-5′-monophosphate (8-oxo-dGMP) and pyrophosphate (PPi). The amount of generated pyrophosphate (PPi) is then measured by a bioluminescent reaction. In detail: The MTH1 (human mutT homologue 1) enzymatic assay is performed as a luminescence-based 384-well assay. In a first step, purified human recombinant MTH1 (human MTH1, residues 42-197, Uniprot-ID: P36639, expressed in E. coli) is incubated in assay buffer for 20 minutes at 22° C. with test compound at different concentrations or without test compound (as a negative control). The assay buffer contains 100 mM Tris-acetate pH 7.5, 40 mM NaCl, 10 mM Mg(OAc)₂, 0.005% Tween 20 and 2 mM dithiothreitol (DTT). An Echo 555 (Labcyte) is used for dispensing of compound solutions. Then, in a second step, the substrate 8-oxo-dGTP is added and the reaction mixture is incubated for 30 minutes at 22° C. The pharmacologically relevant assay volume is 5 PI. The final concentrations in the assay during incubation of the reaction mixture are 0.1-0.2 nM, typically 0.1 nM, MTH1 and 6 μM 8-oxo-dGTP. After incubation of the reaction mixture, the generation of pyrophosphate (PPi) as a result of nucleotide triphosphate (8-oxo-dGTP) hydrolysis by MTH1 is detected using the PPiLight inorganic pyrophosphate assay kit (Lonza) (third step). 2 to 5 μl, such as 3 to 5 μl, typically 3 or 4 μl, for instance 4 μl, of a 1:1 mixture of PPiLight converting reagent (AMP) and PPiLight detection reagent are added to the reaction mixture. In the presence of pyrophosphate, the detection reagent catalyzes the conversion of AMP to ATP. Luciferase then produces light from the newly formed ATP and luciferin. The amount of light produced is directly proportional to the amount of PPi in the sample. Following signal development for 1 h the plates are analysed in an EnVision (PerkinElmer) microplate reader using the ultra sensitive luminescence option. Data are processed employing the Genedata Screener software. In particular, IC₅₀ values are determined in the usual manner (using the Hill Equation) by fitting a dose-response curve to the data points using nonlinear regression analysis.

Hill Equation: Y=S₀+(S_{inf}−S₀)/(1+(10^{logIC}₅₀/10^{c})ⁿ); Y: luminescence signal (response), S₀: activity level at zero concentration of test compound, Si_{nf}=activity level at infinite concentration, Hill coefficient n=measure of the slope at IC₅₀, c=concentration in logarithmic units corresponding to the values on the x-axis of the dose response curve plot

IC₅₀=half maximal inhibitory concentration
8-oxo-dGTP=8-Oxo-2′-deoxyguanosine-5′-triphosphate
8-oxo-dG MP=8-Oxo-2′-deoxyguanosine-5′-monophosphate
Tris=Tris(hydroxymethyl)-aminomethan
OAc=Acetoxy group
PPi=pyrophosphate
AMP=adenosine monophosphate
ATP=adenosine triphosphate

### Measurements of Binding Affinities and Kinetics on MTH1 Surfaces by Surface Plasmon Resonance (SPR)

The SPR experiments were performed using a Biacore4000 instrument. Recombinant in E. coli cells produced humanMTH1 (aminoacids 42 to 197; His tag at the N-terminus; sequence: MGSSHHHHHHSSGLVPRGSHMGASRLYTLVLVLQPQRVLLGMKKRGFGA GRWNGFGGKVQEGETIEDGARRELQEESGLTVDALHKVGQIVFEFVGEPE LMDVHVFCTDSIQGTPVESDEMRPCWFQLDQIPFKDMWPDDSYWFPLLLQ KKKFHGYFKFQGQDTILDYTLREVDTV; size: ˜20 kDa including tags) was purchased from Abnova (Cat#ab99390). HumanMTH1 was immobilized on Biacore CM5 chips at 25° C. and a flow rate of 10 μL/min using amine coupling at pH 5.5 and in presence of 1 μM MSC2567771B-1 according to Biacore's standard protocol. MTH1 was applied at a concentration of 10 μg/mL and depending on the duration of the injection time immobilization levels between 500 and 2,000 RU were obtained. Sample compounds were applied in form of titration series with a doubling of concentration at each subsequent injection. In general, 10 concentrations were injected covering a dilution range of 500 fold. Before and after each of these titration series the binding capability of the surface was controlled by the injection of the positive control S-Crizotinib at a fixed concentration of 2 μM. Kinetic titration experiments were performed at 25° C. with a flow rate of 30 μL/min, a sample contact time of 90 sec and a dissociation time of 420 sec in running buffer (20 mM Tris-HCl (Tris(hydroxymethyl)-aminomethane HCl) pH 7.40, 150 mM NaCl, 5 mM MgCl₂, 1 mM DTT (dithiothreitol), 0.1 mM EGTA (ethylene glycol tetraacetic acid), 0.05% Tween 20) containing 2% DMSO (dimethyl sulfoxide). Buffer injections identical to the sample injections were executed at the beginning of the successive series for the purpose of double referencing. Solvent correction cycles (eight correction points, 1.4%-2.8% DMSO) were run at the same intervals. For surface conditioning ten start-up cycles (buffer injections) were run. Data points were collected at a sample rate of 10 Hz.

Data sets were processed and analyzed using the software Biacore4000 Evaluation, version 1.0. Solvent corrected and double-referenced association and dissociation phase data were fitted to a simple 1:1 interaction model with mass transport limitations.

Measured values are indicated as KD IC₅₀ in the Table below.

### ¹H NMR

¹H NMR was recorded on Bruker DPX-300, DRX-400, AVII-400 or on a 500 MHz spectrometer, using residual signal of deuterated solvent as internal reference. Chemical shifts (6) are reported in ppm relative to the residual solvent signal (b=2.49 ppm for ¹H NMR in DMSO-d₆). ¹H NMR data are reported as follows: chemical shift (multiplicity, coupling constants, and number of hydrogens). Multiplicity is abbreviated as follows: s (singlet), d (doublet), t (triplet), q (quartet), m (multiplet), br (broad).

### LC-MS

Liquid chromatography retention times were obtained using a Chromolith RP-18e 50-4.6 mm column and the following conditions:

Solvents: A: H₂O+0.05% HCOOH|B: MeCN+0.04% HCOOH

Gradient: Solvent B: 0 to 2.8 min: increase from 4% to 100%; 2.8 to 3.3 min: 100% B

Flow: 2.4 ml/min

### Measurement of Solubility by Shake Flask Solubility Measurement

Compound solubility in aqueous solution is determined by a standard method in the art, the shake flask method. Solubility measurement is performed under equilibrium conditions at pH 7.4. Samples are prepared by dissolving an excess of the test compound in a phosphate buffer (made up of 3,954 g sodium hydrogenphosphate-monohydrate, 6,024 g sodium chloride, 950 ml ultrapure water and adjusted to pH 7.4 with 0.1 M NaOH or 0.1 M HCl). These samples are shaken at 37° C. with 450 rpm (shaker: TiMix control Bühler) for 24 h in total until equilibrium is reached. After 7 hours shaking, the pH value of the sample is controlled and adjusted, if necessary. It is also checked that the sample (test compound) is available in excess. Shortly before the end of 24 h, samples are checked again for pH and precipitate. At the end of 24 h and after separation of the solid by filtration, the concentration of the compound in the filtrate is determined by liquid chromatography using US detection (LC-UV).

Sample preparation: Incubation hood: TH 15 Bühler; pH device 766 Calimatic Knick; pH electrode InLab 423 Mettler

Chromatography Conditions:

Column: Chromolith RP18e 100×3 mm

Wavelength range: 190-400 nm
select a suitable evaluation wavelength

Injection volume: Probe 5 μl and 15 μl; Standard 5 μl

Column stove temperature: 37° C.

LC Eluent Preparation:

Eluent A: water/formic acid (999:1; v/v)

Eluent B: acetonitrile/formic acid (999:1; v/v)

Gradient:

| Time [Min] | Eluent A [%] | Eluent B [%] | Flow [mL/min] |
|---|---|---|---|
| 0 | 90 | 10 | 0.85 |
| 0.6 | 90 | 10 | 0.85 |
| 4 | 10 | 90 | 0.85 |
| 5.5 | 10 | 90 | 0.85 |
| 5.51 | 90 | 10 | 2.5 |
| 8 | 90 | 10 | 2.5 |

### Test Method Microsomal Stability (Intrinsic Clearance)

A microsomal stability assay is used to measure in vitro clearance (Clint). The assay involves measuring the rate of disappearance of a compound due to its intrinsic attitude to be metabolized (“intrinsic” meaning that the disappearance is not affected by other properties like permeability, binding etc. that play a role when quantifying in vivo clearance). The microsomal stability (intrinsic clearance, Clint) and thus metabolic stability is generally given as μl/min/mg protein. It can be visualized as the volume of solution that 1 mg of microsomes is able to clear of the compound in one minute.

### Instrumentation

A Tecan Genesis workstation (RSP 150/8) was used for to perform the microsomal incubations. Analysis was carried out using a Waters ACQUITY UPLC system coupled to an ABSciex API3000 mass spectrometer. Data analysis was performed using Assay Explorer (Symyx).

### UPLC Conditions

Column: Acquity UPLC BEH C18, 2.1×50 mm, 1.7 μm (Waters) Mobile phases: A=0.1% formic acid in water; B=acetonitrile

| Gradient Time [Min] | % A | % B |
|---|---|---|
| initial | 90 | 10 |
| 0.47 | 5 | 95 |
| 0.65 | 5 | 95 |
| 0.66 | 90 | 10 |

Flow rate: 0.750 mL/min; Detection: ESI, MRM; Injection: 10 μL; Column temperature: 50° C.

### Chemicals

Potassium phosphate buffer: 0.05 M potassium phosphate buffer pH 7.4 containing 1 mM MgCl₂
NADPH (nicotinamide adenine dinucleotide phosphate): 22.5 mg NADPH-Na₄ in 1.8 ml potassium phosphate buffer
Acetonitrile: 50 Vol % acetonitrile (1 volume acetonitrile, 1 volume water)
DMSO: 20 Vol % DMSO in water
Stock solution of 20 mg/ml human or mouse liver microsomes (protein)/ml in phosphate buffer
Stock solution of 10 mM compound in 100% DMSO

### Microsomal Incubation

Dilution of test compounds was done in 2 steps starting from a 10 mM stock solution of the respective compound in 100% DMSO. First 4 μl stock solution was added to 196 μl of 20 Vol % DMSO. In a second step, 10 μl of the first dilution were added to 1590 μl potassium phosphate buffer to achieve a final concentration of 1.25 μM in the final compound dilution. Thus, the amount of organic solvent in the assay was kept to a minimum (<1%).

The human or mouse liver microsome (protein) solution to be used in the assay was prepared by mixing 750 μl stock solution (20 mg/ml) and 2250 μl potassium phosphate buffer to a final concentration of 5 mg/ml.

Incubation was carried out on a 96 deep well incubation plate. 160 μl per well of the final compound dilution were transferred onto the incubation plate. Four samples of each compound dilution were assayed. 20 μl/well liver microsome solution was added to each well and the samples were then preincubated for 5 min at 37° C. and 800 rpm agitation. Two reference compounds (verapamil and dextromethorphan) were used in parallel in every experiment and for each species (human or mouse microsomes) to ensure system performance and for comparison.

On a separate stop plate, 160 μl acetonitrile were added per well.

After preincubation, i.e. at time t₁=0 minutes, 18 μl samples of incubated compound solution and were transferred and added per well (containing acetonitrile) on the stop plate to prevent a reaction (0 minutes control samples, 4 samples per compound). Equally, 18 μl samples of incubated reference compound solution were transferred and added per well (containing acetonitrile) on the stop plate at time t₁=0 minutes and again after 30 minutes (t₄), solubility and chemical stability of the compound were checked.

To start the reaction, 26 μl NADPH solution (cofactor) was added to all wells comprising preincubated compound dilution or reference solution with the exception of those wells comprising preincubated compound dilution that were to be used as the 30 minutes control samples, where 26 μl phosphate buffer were added instead. Incubation was then continued at 37° C. and 800 rpm agitation.

In the final assay solutions (i.e. in each well comprising solution of compound, microsomes (protein) and NADPH respectively phosphate buffer), the final protein concentration was 0.5 mg/ml and the compound concentration 1 mg/ml.

After t₂=5 minutes, t₃=10 minutes and t₄=20 minutes of incubation time (i.e. after start of the reaction), 20 μl samples of incubated compound solution (4 samples per compound) and reference compound solution were transferred and added per well of acetonitrile on the stop plate.

After t₄=30 minutes of incubation time, 20 μl samples of incubated compound solution (4 samples per compound) and 20 μl samples of the 30 minutes control samples (containing buffer instead of NADPH) as well as 20 μl samples of incubated reference compound solution were transferred and added per well of acetonitrile on the stop plate.

The quenched samples were centrifuged at 4000 g for 1 h at 4° C. 80 μl of the supernatant were transferred into 96 well plates for analysis by LC-MS/MS.

### Data Analysis

The microsomal/metabolic stability of a compound was determined by measurement of the change in LC-MS/MS peak area over time. Data are fitted according to a log linear model in line with Michaelis/Menten. The Clint value is calculated from the slope (k) of the linear log transformed concentration per time plot divided by the amount of microsomes (0.5 mg/ml): Clint (μl/min/mg protein)=k*1000/protein concentration. Assay Explorer software was used to automatically calculate the slope k of the decline.

In the following Table 1, IC50 and KD IC50 values are grouped as follows: A:≤1 nM; 1 nM<B≤100 nM; C>100 nM.

It is noted that the following Table 1 illustrates that compounds according to the present invention have excellent inhibitory properties, with numerous examples exhibiting IC50 values (enzymatic assay) in the lower picomolar range, and unprecedented interaction with the target (incl residence time), as will be further set out in Table 3.

TABLE 1
Exemplary compounds

| | | | | | LC-MS |
|---|---|---|---|---|---|
| | | | | | RT |
| Ex | Structure | | | KD | (min): |
| No | Name | ¹H NMR | IC₅₀ | IC₅₀ | [M + H⁺] |
| 1 | 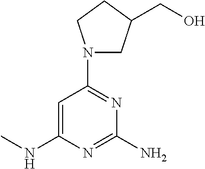 [SMILES: CNc1cc(N2CCC(CO)C2)nc(N)n1] [1-(2-amino-6-methylamino- pyrimidin-4-yl)-pyrrolidin-3-yl]- methanol | — | A | | 1.121 [224.1] |
| 2 | 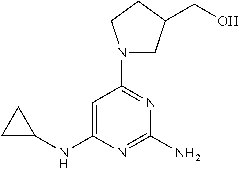 [SMILES: Nc1nc(NC2CC2)cc(N2CCC(CO)C2)n1] [1-(2-amino-6-cyclopropyl amino-pyrimidin-4-yl)- pyrrolidin-3-yl]-methanol | ¹H NMR (500 MHz, DMSO) δ 6.40 (s, 1H), 5.52 (s, 2H), 4.95 (s, 1H), 3.31- 3.48 (m, 4H), 3.28 (dd, J = 17.4, 7.6 Hz, 1H), 3.08 (dd, J = 9.8, 6.9 Hz, 1H), 2.41 (br. m, 1H), 2.36-2.30 (m, 1H), 1.97-1.91 (m, 1H), 1.62- 1.61 (m, 1H), 0.70- 0.52 (m, 2H), 0.49-0.33 (m, | A | | 1.262 [250.1] |
| | | 2H). | | | |
| 3 | 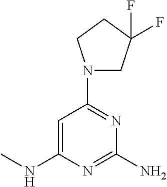 [SMILES: CNc1cc(N2CCC(F)(F)C2)nc(N)n1] 6-(3,3-difluoro-pyrrolidin-1- yl)-N⁴-methyl- pyrimidine-2,4-diamine | ¹H NMR (400 MHz, DMSO) δ 6.17 (d, J = 4.8 Hz, 1H), 5.57 (s, 2H), 4.77 (s, 1H), 3.71 (t, J = 13.4 Hz, 2H), 3.50 (t, J = 7.3 Hz, 2H), 2.68 (d, J = 4.9 Hz, 2H), 2.50 (m, 2H), 2.49-2.39 (m, 2H). ¹⁹F NMR (377 MHz, DMSO) δ −100.19 (s). | A | | 1.291 [230.1] |
| 4 | 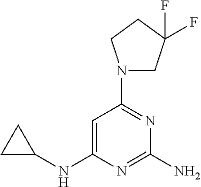 [SMILES: Nc1nc(NC2CC2)cc(N2CCC(F)(F)C2)n1] N⁴-cyclopropyl-6-(3,3- difluoro-pyrrolidin-1-yl)- pyrimidine-2,4-diamine | ¹H NMR (400 MHz, DMSO) δ 11.04 (s, 1H), 8.00 (s, 1H), 7.42 (s, 2H), 5.15 (s, 1H), 3.89 (t, J = 12.9 Hz, 2H), 3.67 (s, 2H), 2.55 (d, J = 6.2 Hz, 2H), 0.83 (q, J = 6.6 Hz, 2H), 0.61-0.49 (m, 2H). | B | | |
| 5 | 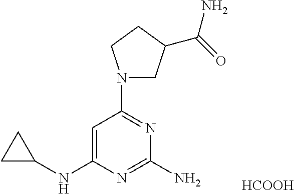 [SMILES: NC(=O)C1CCN(c2cc(NC3CC3)nc(N)n2)C1 ; "HCOOH"] 1-(2-amino-6-cyclopropyl amino-pyrimidin-4-yl)- pyrrolidine-3-carboxylic acid amide-formate | ¹H NMR (300 MHz, DMSO) δ 8.15 (s, 1H), 7.45 (s, 1H), 6.93 (s, 1H), 6.76 (s, 1H), 5.97 (s, 2H), 5.00 (s, 1H), 3.54 (m, 3H), 3.00 (m, 3H), 2.06 (m, 2H), 0.74-0.60 (m, 2H), 0.51- 0.37 (m, 2H). | B | | 1.164 [263.0] |
| 6 | 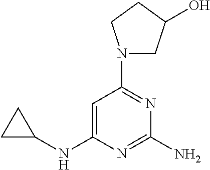 [SMILES: Nc1nc(NC2CC2)cc(N2CCC(O)C2)n1] 1-(2-amino-6-cyclopropylami no-pyrimidin-4-yl)-pyrrolidin- 3-ol | ¹H NMR (400 MHz, DMSO) δ 6.20 (s, 1H), 5.34 (s, 2H), 4.95 (s, 1H), 4.87 (d, J = 3.3 Hz, 1H), 4.32 (s, 1H), 4.08 (q, J = 5.2 Hz, 1H), 3.39 (m, 2H), 3.18 (m, 3H), 2.42 (d, J = 2.9 Hz, 1H), 2.01- 1.88 (m, 1H), 1.83 (s, 1H), 0.64 (q, J = 6.3 Hz, 2H), 0.41 (d, J = 2.7 Hz, 2H). | B | | 1.167 [236.1] |
| 7 | 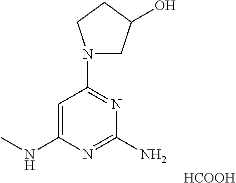 1-(2-amino-6-methylamino- pyrimidin-4-yl)-pyrrolidin-3-ol- formate | ¹H NMR (400 MHz, DMSO) δ 8.18 (s, 1H), 6.13 (s, 1H), 5.60 (s, 2H), 4.71 (s, 1H), 4.30 (s, 1H), 3.36 (dt, J = 13.5, 6.7 Hz, 4H), 3.22 (d, J = 9.8 Hz, 2H), 2.68 (d, J = 4.8 Hz, 3H), 1.99- 1.86 (m, 1H), 1.82 (s, 1H). | B | | 0.832 [210.1] |
| 8 | 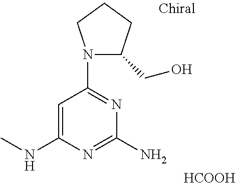 [SMILES: CNc1cc(N2CCC[C@@H]2CO)nc(N)n1 ; "HCOOH"] [(R)-1-(2-amino-6- methylamino-pyrimidin-4-yl)- pyrrolidin-2-yl]-methanol- formate | ¹H NMR (400 MHz, DMSO) δ 8.23 (s, 1H), 6.11 (d, J = 4.4 Hz, 1H), 5.49 (s, 2H), 4.78 (s, 1H), 3.98 (s, 2H), 3.49 (dd, J = 10.3, 5.5 Hz, 2H), 3.30 (dd, J = 10.3, 6.4 Hz, 2H), 3.24- 3.14 (m, 1H), 2.69 (d, J = 4.8 Hz, 3H), 1.88 (m, 3H). | B | | 1.143 [224.1] |
| 9 | 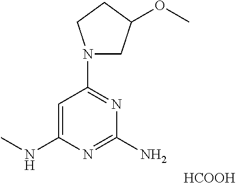 86-(3-methoxy-pyrrolidin-1-yl)- N⁴-methyl-pyrimidine-2,4- diamine-formate | ¹H NMR (400 MHz, DMSO) δ 8.17 (s, 1H), 6.09 (d, J = 4.9 Hz, 1H), 5.55 (s, 2H), 4.72 (s, 1H), 3.98 (dd, J = 7.3, 3.7 Hz, 1H), 3.39 (dd, J = 9.3, 5.3 Hz, 3H), 3.27 (t, J = 7.0 Hz, 1H), 3.24 (s, 3H), 2.68 (d, J = 4.9 Hz, 3H), 1.97 (td, J = 8.3, 4.4 Hz, 2H). | A | | 1.182 [224.0] |
| 10 | 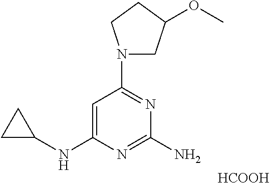 [SMILES: COC1CCN(c2cc(NC3CC3)nc(N)n2)C1 ; "HCOOH"] N⁴-cyclopropyl-6-(3- methoxy-pyrrolidin-1-yl)-pyri midine-2,4-diamine- formate | ¹H NMR (400 MHz, DMSO) δ 8.19 (s, 1H), 6.46 (s, 1H), 5.59 (s, 2H), 4.97 (s, 1H), 4.03-3.97 (m, 1H), 3.41 (d, J = 3.0 Hz, 3H), 3.33- 3.26 (m, 1H), 3.25 (s, 3H), 2.42 (dd, J = 6.3, 3.4 Hz, 1H), 2.04- 1.92 (m, 2H), 1.10 (d, J = 6.1 Hz, 1H), 0.72-0.57 (m, 2H), 0.50-0.33 (m, 2H). | B | | 1.326 [250.1] |
| 11 | 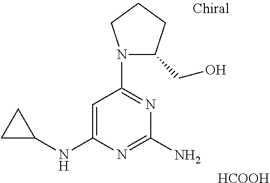 [SMILES: Nc1nc(NC2CC2)cc(N2CCC[C@@H]2CO)n1 ; "HCOOH"] [(R)-1-(2-amino-6- cyclopropylamino-pyrimidin- 4-yl)-pyrrolidin-2-yl]-methanol- formate | 1H NMR (400 MHz, DMSO) δ 8.23 (s, 1H), 6.44 (s, 1H), 5.52 (s, 2H), 5.02 (s, 1H), 4.00 (s, 1H), 3.50 (dd, J = 10.3, 5.4 Hz, 1H), 3.39 (dd, J = 14.0, 7.0 Hz, 1H) 3.31 (dd, J = 10.1, 6.7 Hz, 2H), 3.21 (d, J = 7.0 Hz, 1H), 2.42 (d, J = 2.8 Hz, 1H), 1.99-1.78 (m, 4H), 1.09 (t, J = 6.9 Hz, 1H), 0.71- 0.56 (m, 2H), 0.49- | B | | 1.311 [250.1] |
| | | 0.31 (m, 2H). | | | |
| 12 | 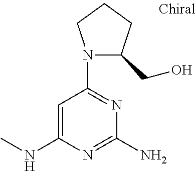 [SMILES: CNc1cc(N2CCC[C@H]2CO)nc(N)n1] [(S)-1-(2-amino-6- methylamino-pyrimidin-4-yl)- pyrrolidin-2-yl]-methanol | ¹H NMR (400 MHz, DMSO) δ 8.18 (s, 1H), 6.12 (d, J = 4.9 Hz, 1H), 5.52 (s, 2H), 4.76 (s, 1 H), 3.97 (s, 1 H), 3.47 (dd, J = 10.3, 5.5 Hz, 1H), 3.30 (m, 2H), 3.18 (m, 2H), 2.68 (d, J = 4.8 Hz, 3H), 1.84 (m, 3H). | B | | 1.075 [224.1] |
| 13 | 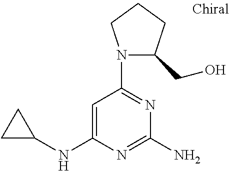 [SMILES: Nc1nc(NC2CC2)cc(N2CCC[C@H]2CO)n1] [(S)-1-(2-amino-6- cyclopropylamino-pyrimidin- 4-yl)-pyrrolidin-2-yl]- methanol | ¹H NMR (400 MHz, DMSO) δ 7.91 (s, 1H), 7.34 (s, 1H), 5.17 (s, 1H), 3.86- 3.04 (m, 5H), 2.60- 2.54 (m, 1H), 1.96 (m, 6H), 0.81 (d, J = 5.5 Hz, 2H), 0.54 (m, 2H). | B | | 1.300 [250.1] |
| 14 | 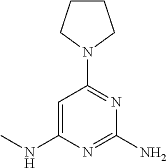 N⁴-methyl-6-pyrrolidin-1-yl- pyrimidine-2,4- diamine | | | | |
| 15 | 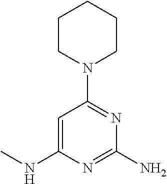 N⁴-methyl-6-piperidin-1-yl- pyrimidine-2,4- diamine | | | | |
| 16 | 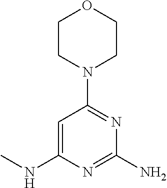 N⁴-methyl-6-morpholin-4-yl- pyrimidine-2,4- diamine | | | | |
| | 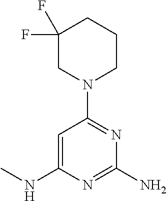 [SMILES: CNc1cc(N2CCCC(F)(F)C2)nc(N)n1] 6-(3,3-difluoro- piperidin-1-yl)-N⁴-methyl- pyrimidine-2,4-diamine | | | | |
| 17 | 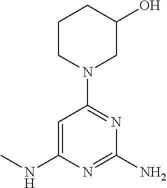 [SMILES: CNc1cc(N2CCCC(O)C2)nc(N)n1] 1-(2-amino-6-methylamino- pyrimidin-4-yl)- piperidin-3-ol | | | | |
| 18 | 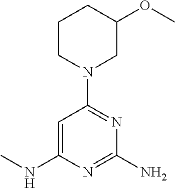 6-(3-methoxy-piperidin-1-yl)- N⁴-methyl-pyrimidine-2,4- diamine | | | | |
| 19 | 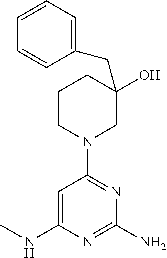 [SMILES: CNc1cc(N2CCCC(O)(Cc3ccccc3)C2)nc(N)n1] 1-(2-amino-6-methylamino- pyrimidin-4-yl)- 3-benzyl-piperidin-3-ol | | | | |
| 20 | 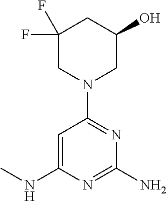 [SMILES: CNc1cc(N2C[C@H](O)CC(F)(F)C2)nc(N)n1] (R)-1-(2-amino-6-methylami no-pyrimidin-4-yl)-5,5-difluoro- piperidin-3-ol | | | | |
| 21 | 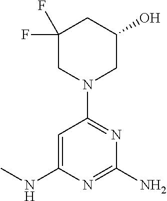 [SMILES: CNc1cc(N2C[C@@H](O)CC(F)(F)C2)nc(N)n1] (S)-1-(2-amino-6-methylami no-pyrimidin-4-yl)-5,5-difluoro- piperidin-3-ol | | | | |
| 22 | 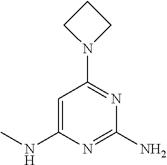 6-azetidin-1-yl-N⁴-methyl- pyrimidine-2,4-diamine | | | | |
| 23 | 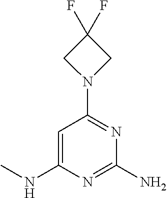 6-(3,3-difluoro-azetidin-1-yl)-N⁴- methyl-pyrimidine-2,4-diamine | | | | |
| 24 | 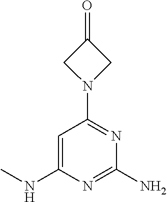 1-(2-amino-6-methylamino- pyrimidin-4-yl)-azetidin-3-one | | | | |
| 25 | 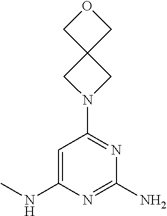 [SMILES: CNc1cc(N2CC3(COC3)C2)nc(N)n1] N⁴-methyl-6-(2-oxa-6-aza-spiro [3.3]hept-6-yl)- pyrimidine-2,4-diamine | | | | |
| 26 | 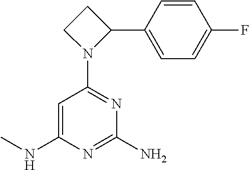 [SMILES: CNc1cc(N2CCC2c2ccc(F)cc2)nc(N)n1] 6-[2-(4-fluoro-phenyl)- azetidin-1-yl]-N⁴- methyl-pyrimidine-2,4-diamine | | | | |
| 27 | 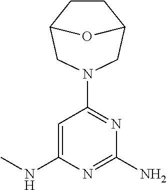 [SMILES: CNc1cc(N2CC3CCC(C2)O3)nc(N)n1] N⁴-methyl-6-(8-oxa-3- azabicyclo[3.2.1]octan-3- yl)pyrimidine-2,4-diamine | | | | |
| 28 | 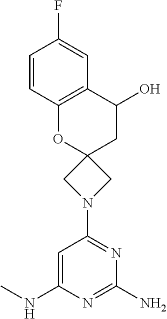 [SMILES: CNc1cc(N2CC3(CC(O)c4cc(F)ccc4O3)C2)nc(N)n1] 1-[2-amino-6- (methylamino)pyrimidin-4-yl]- 6′-fluoro-spiro[azetidine-3,2′- chromane]-4′-ol | | | | |
| 29 | 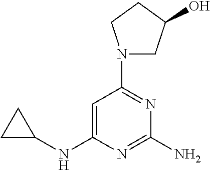 [SMILES: Nc1nc(NC2CC2)cc(N2CC[C@@H](O)C2)n1] (R)-1-(2-Amino-6- cyclopropylamino-pyrimidin-4- yl)-pyrrolidin-3-ol | ¹H NMR (400 MHz, DMSO-d6) δ 6.19 (d, J = 2.5 Hz, 1H), 5.33 (s, 2H), 4.94 (s, 1H), 4.84 (d, J = 3.6 Hz, 1H), 4.31 (d, J = 3.6 Hz, 1 H), 4.05 (q, J = 5.2 Hz, 1H), 3.23 (d, J = 11.1 Hz, 1H), 3.17 (d, J = 5.1 Hz, 3H), 2.40 (m, 1H), 1.93 (m, 1H), 1.82 (s, 1H), 0.62 (m, 2H), 0.47- | B | B | 1.094 [236.1] |
| | | 0.35 (m, 2H). | | | |
| 30 | 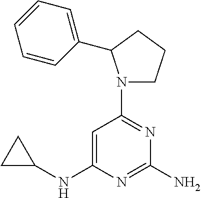 [SMILES: Nc1nc(NC2CC2)cc(N2CCCC2c2ccccc2)n1] N4-Cyclopropyl-6-(2-phenyl- pyrrolidin-1-yl)-pyrimidine-2,4- diamine | 1H NMR (400 MHz, DMSO-d6) δ 7.29 (t, J = 7.5 Hz, 2H), 7.22-7.14 (m, 3H), 6.14 (s, 1H), 5.28 (s, 2H), 4.92 (s, 1H), 4.82 (s, 1H), 3.67 (s, 1H), 3.57 (d, J = 8.7 Hz, 1H), 2.33 (s, 1H), 2.20 (s, 1H), 1.93-1.72 (m, 3H), 0.53 (s, 1H), 0.45-0.27 (m, 2H). | B | B | 1.655 [296.2] |
| 31 | 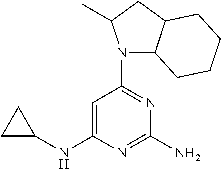 [SMILES: CC1CC2CCCCC2N1c1cc(NC2CC2)nc(N)n1] N4-Cyclopropyl-6-(2-methyl- octahydro-indol-1-yl)- pyrimidine-2,4-diamine | | B | B | 1.895 [288.2] |
| 32 | 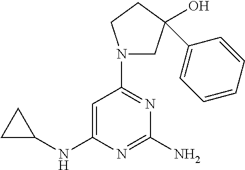 [SMILES: Nc1nc(NC2CC2)cc(N2CCC(O)(c3ccccc3)C2)n1] 1-(2-Amino-6- cyclopropylamino-pyrimidin-4- yl)-3-phenyl-pyrrolidin-3-ol | 1H NMR (500 MHz, DMSO-d6) δ 7.57-7.47 (m, 2H), 7.36 (dd, J = 8.4, 7.0 Hz, 2H), 7.32-7.22 (m, 1H), 6.31 (s, 1H), 5.46 (s, 2H), 5.36 (s, 1H), 4.98 (s, 1H), 3.52 (m, 2H), 2.42 (m, 1H), 2.28 (dt, J = 12.3, 9.3 Hz, 1H), 2.17- 2.07 (m, 1H), 0.64 (m, 2H), 0.40 (m, | B | B | 1.530 [312.2] |
| | | 2H). | | | |
| 33 | 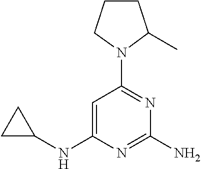 [SMILES: CC1CCCN1c1cc(NC2CC2)nc(N)n1] N4-Cyclopropyl-6-(2-methyl- pyrrolidin-1-yl)-pyrimidine-2,4- diamine | 1H NMR (500 MHz, DMSO-d6) δ 6.20 (d, J = 2.5 Hz, 1H), 5.31 (s, 2H), 4.96 (s, 1H), 4.04 (s, 1H), 3.35 (m, 1H), 3.26-3.18 (m, 1H), 2.40 (m, 1H), 2.02-1.89 (m, 2H), 1.89- 1.80 (m, 1H), 1.59 (m, 1H), 1.12 (d, J = 6.3 Hz, 3H), 0.68- 0.54 (m, 2H), 0.46-0.33 (m, | B | B | 1.447 [234.2] |
| | | 2H). | | | |
| 34 | 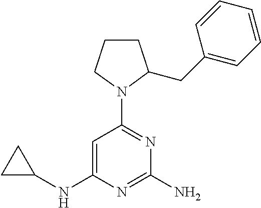 [SMILES: Nc1nc(NC2CC2)cc(N2CCCC2Cc2ccccc2)n1] 6-(2-Benzyl-pyrrolidin-1-yl)-N4- cyclopropyl-pyrimidine-2,4- diamine | 1H NMR (500 MHz, DMSO-d6) δ 7.35-7.24 (m, 4H), 7.20 (m, 1H), 6.27 (s, 1H), 5.39 (s, 2H), 5.08 (s, 1H), 3.34 (s, 1H), 3.23 (m, 1H), 3.13- 3.04 (m, 1H), 2.53 (m, 1H), 2.45 (m, 1H), 1.89- 1.77 (m, 2H), 1.71 (m, 2H), 0.64 (m, 2H), 0.42 (m, 2H). | A | B | 1.806 [310.2] |
| 35 | 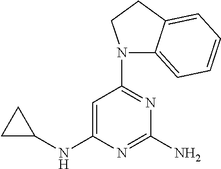 [SMILES: Nc1nc(NC2CC2)cc(N2CCc3ccccc32)n1] N4-Cyclopropyl-6-(2,3-dihydro- indol-1-yl)-pyrimidine-2,4- diamine | 1H NMR (400 MHz, DMSO-d6) δ 8.41 (d, J = 7.9 Hz, 1H), 7.14 (dd, J = 7.3, 1.2 Hz, 1H), 7.10-7.02 (m, 1H), 6.80 (td, J = 7.3, 1.1 Hz, 1H), 6.56 (d, J = 2.6 Hz, 1H), 5.71 (s, 2H), 5.27 (s, 1H), 3.90 (t, J = 8.7 Hz, 2H), 3.12 (t, J = 8.6 Hz, 2H), 0.67 (td, J = 6.8, 4.5 Hz, 2H), 0.48-0.39 (m, 2H). | B | B | 1.687 [268.1] |
| 36 | 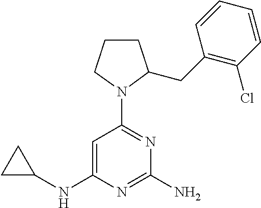 [SMILES: Nc1nc(NC2CC2)cc(N2CCCC2Cc2ccccc2Cl)n1] 6-[2-(2-Chloro-benzyl)- pyrrolidin-1-yl]-N4-cyclopropyl- pyrimidine-2,4-diamine | 1H NMR (400 MHz, DMSO-d6) δ 7.43 (m, 2H), 7.27 (m, 2H), 6.24 (s, 1H), 5.35 (s, 2H), 5.14 (s, 1H), 3.41 (t, J = 9.1 Hz, 1H), 3.21 (dd, J = 13.1, 4.0 Hz, 1H), 2.72 (dd, J = 13.1, 9.8 Hz, 1H), 2.40 (dt, J = 6.9, 3.5 Hz, 1H), 1.70 (m, 2H), 0.71- 0.58 (m, 2H), 0.47-0.38 (m, 2H). | A | | 1.931 [344.1] |
| 37 | 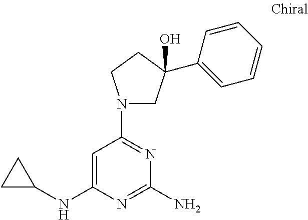 [SMILES: Nc1nc(NC2CC2)cc(N2CC[C@](O)(c3ccccc3)C2)n1] (S)-1-(2-Amino-6- cyclopropylamino-pyrimidin-4- yl)-3-phenyl-pyrrolidin-3-ol | | B | | 1.512 [312.2] |
| 38 | 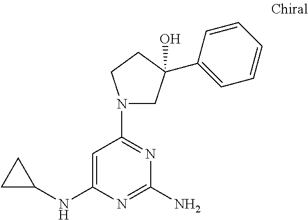 [SMILES: Nc1nc(NC2CC2)cc(N2CC[C@@](O)(c3ccccc3)C2)n1] (R)-1-(2-Amino-6- cyclopropylamino-pyrimidin-4- yl)-3-phenyl-pyrrolidin-3-ol | | B | | |
| 39 | 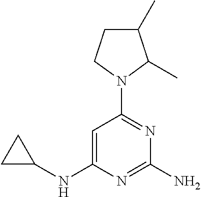 [SMILES: CC1CCN(c2cc(NC3CC3)nc(N)n2)C1C] N4-Cyclopropyl-6-(2,3- dimethyl-pyrrolidin-1-yl)- pyrimidine-2,4-diamine | 1H NMR (400 MHz, DMSO-d6) δ 6.20 (s, 1H), 5.30 (d, J = 5.8 Hz, 2H), 4.96 (d, J = 4.8 Hz, 1H), 3.47-3.35 (m, 1H), 2.41 (m, 1H), 2.08 (m, 1H), 1.94 (s, 1H), 1.49 (m, 1H), 1.17 (m, 2H), 0.99 (m, 4H), 0.63 (m, 2H), 0.40 (m, 2H). | B | | 1.584 [248.4] |
| 40 | 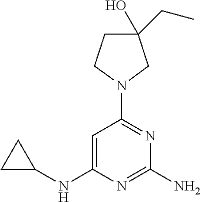 [SMILES: CCC1(O)CCN(c2cc(NC3CC3)nc(N)n2)C1] 1-(2-Amino-6- cyclopropylamino-pyrimidin-4- yl)-3-ethyl-pyrrolidin-3-ol | 1H NMR (400 MHz, DMSO-d6) δ 6.19 (d, J = 2.5 Hz, 1H), 5.33 (s, 2H), 4.94 (s, 1H), 4.49 (s, 1H), 3.53-3.34 (m, 2H), 3.16 (d, J = 10.8 Hz, 1H), 2.41 (dq, J = 6.9, 3.2 Hz, 1H), 1.88- 1.71 (m, 2H), 1.60 (qd, J = 7.2, 2.6 Hz, 2H), 0.94 (t, J = 7.4 Hz, 3H), 0.64 (m, 2H), 0.42- 0.37 (m, 2H). | B | | 1.281 [264.3] |
| 41 | 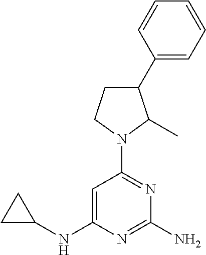 [SMILES: CC1C(c2ccccc2)CCN1c1cc(NC2CC2)nc(N)n1] N4-Cyclopropyl-6-(2-methyl-3- phenyl-pyrrolidin-1-yl)- pyrimidine-2,4-diamine | | B | | 1.809 1.827 [310.2] |
| 42 | 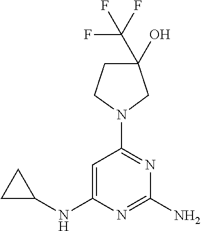 [SMILES: Nc1nc(NC2CC2)cc(N2CCC(O)(C(F)(F)F)C2)n1] 1-(2-Amino-6- cyclopropylamino-pyrimidin-4- yl)-3-trifluoromethyl-pyrrolidin- 3-ol | 1H NMR (400 MHz, DMSO-d6) δ 6.37 (s, 1H), 6.31 (d, J = 2.6 Hz, 1H), 5.44 (s, 2H), 4.98 (s, 1H), 3.55 (s, 3H), 3.42 (td, J = 9.8, 6.9 Hz, 1H), 2.42 (m, 1H), 2.26- 2.13 (m, 1H), 2.10-1.99 (m, 1H), 0.63 (, 2H), 0.40 (dt, J = 6.5, 4.0 Hz, 2H). | B | | 1.3804 [304.2] |
| 43 | 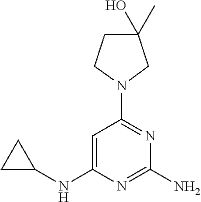 [SMILES: CC1(O)CCN(c2cc(NC3CC3)nc(N)n2)C1] 1-(2-Amino-6- cyclopropylamino-pyrimidin-4- yl)-3-methyl-pyrrolidin-3-ol | 1H NMR (400 MHz, DMSO-d6) δ 6.18 (d, J = 2.5 Hz, 1H), 5.31 (s, 2H), 4.91 (s, 1H), 4.70 (s, 1H), 3.39 (s, 3H), 3.19-3.11 (m, 2H), 2.39 (m, 1H), 1.90-1.73 (m, 2H), 1.30 (s, 3H), 0.62 (m, 2H), 0.43-0.33 (m, 2H). | B | B | 1.182 [250.1] |
| 44 | 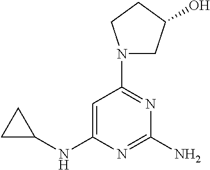 [SMILES: Nc1nc(NC2CC2)cc(N2CC[C@H](O)C2)n1] (S)-1-(2-Amino-6- cyclopropylamino-pyrimidin-4- yl)-pyrrolidin-3-ol | 1H NMR (400 MHz, DMSO-d6) δ 11.03 (s, 1H), 7.83 (s, 1H), 7.28 (s, 2H), 5.09 (s, 2H), 4.38 (s, 1H), 3.79- 3.38 (m, 4H), 1.99 (s, 2H), 0.80 (m, 2H), 0.53 (m, 2H). | B | B | 1.076 [236.1] |
| 45 | 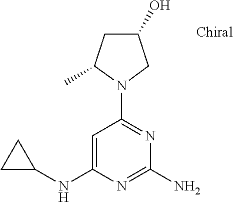 [SMILES: C[C@@H]1C[C@H](O)CN1c1cc(NC2CC2)nc(N)n1] (3S,5R)-1-(2-Amino-6- cyclopropylamino-pyrimidin-4- yl)-5-methyl-pyrrolidin-3-ol | | B | B | 1.195 [250.2] |
| 46 | 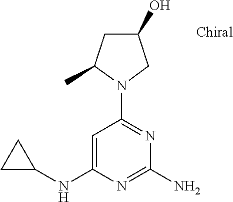 [SMILES: C[C@H]1C[C@@H](O)CN1c1cc(NC2CC2)nc(N)n1] (3R,5S)-1-(2-Amino-6- cyclopropylamino-pyrimidin-4- yl)-5-methyl-pyrrolidin-3-ol | | A | A | |
| 47 | 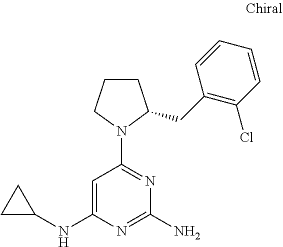 [SMILES: Nc1nc(NC2CC2)cc(N2CCC[C@@H]2Cc2ccccc2Cl)n1] 6-[(R)-2-(2-Chloro-benzyl)- pyrrolidin-1-yl]-N4-cyclopropyl- pyrimidine-2,4-diamine | | A | A | 1.827 [344.1] |
| 48 | 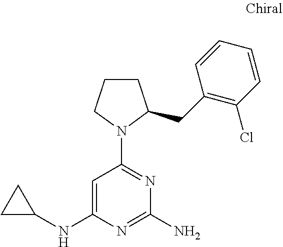 [SMILES: Nc1nc(NC2CC2)cc(N2CCC[C@H]2Cc2ccccc2Cl)n1] 6-[(S)-2-(2-Chloro-benzyl)- pyrrolidin-1-yl]-N4-cyclopropyl- pyrimidine-2,4-diamine | | B | B | 1.851 [344.2] |
| 49 | 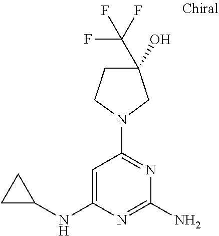 [SMILES: Nc1nc(NC2CC2)cc(N2CC[C@@](O)(C(F)(F)F)C2)n1] (S)-1-(2-Amino-6- cyclopropylamino-pyrimidin-4- yl)-3-trifluoromethyl-pyrrolidin- 3-ol | | A | B | 1.377 [304.1] |
| 50 | 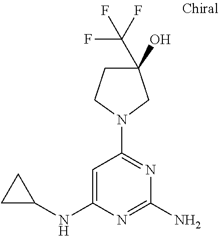 [SMILES: Nc1nc(NC2CC2)cc(N2CC[C@](O)(C(F)(F)F)C2)n1] (R)-1-(2-Amino-6- cyclopropylamino-pyrimidin-4- yl)-3-trifluoromethyl-pyrrolidin- 3-ol | | B | B | 1.381 [304.2] |
| 51 | 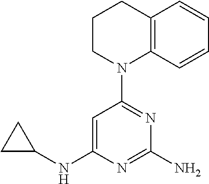 [SMILES: Nc1nc(NC2CC2)cc(N2CCCc3ccccc32)n1] N4-Cyclopropyl-6-(3,4-dihydro- 2H-quinolin-1-yl)-pyrimidine- 2,4-diamine | 1H NMR (400 MHz, DMSO-d6) δ 7.36 (d, J = 8.0 Hz, 1H), 7.16-7.12 (m, 1H), 7.12- 7.07 (m, 1H), 6.93 (td, J = 7.4, 1.2 Hz, 1H), 6.50 (d, J = 2.5 Hz, 1H), 5.61 (d, J = 6.8 Hz, 3H), 3.86-3.78 (m, 2H), 2.73 (t, J = 6.7 Hz, 2H), 1.82 (p, J = 6.6 Hz, 2H), 0.56 (td, J = 6.8, 4.5 Hz, 2H), 0.43-0.33 | A | B | 1.697 [282.1] |
| | | (m, 2H). | | | |
| 52 | 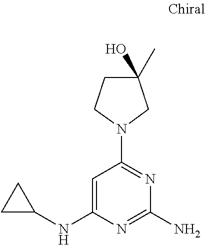 [SMILES: C[C@@]1(O)CCN(c2cc(NC3CC3)nc(N)n2)C1] (R)-1-(2-Amino-6- cyclopropylamino-pyrimidin-4- yl)-3-methyl-pyrrolidin-3-ol | | B | B | 1.186 [250.1] |
| 53 | 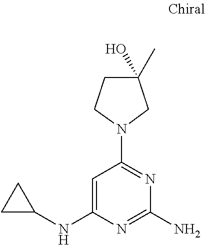 [SMILES: C[C@]1(O)CCN(c2cc(NC3CC3)nc(N)n2)C1] (S)-1-(2-Amino-6- cyclopropylamino-pyrimidin-4- yl)-3-methyl-pyrrolidin-3-ol | | B | B | 1.185 [250.1] |
| 54 | 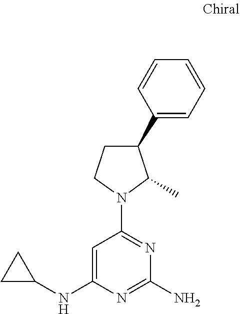 [SMILES: C[C@H]1[C@H](c2ccccc2)CCN1c1cc(NC2CC2)nc(N)n1] N4-Cyclopropyl-6-((2S,3R)-2- methyl-3-phenyl-pyrrolidin-1- yl)-pyrimidine-2,4-diamine | | B | B | 1.795 [310.2] |
| 55 | 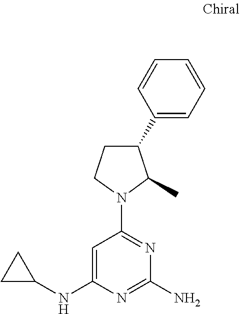 [SMILES: C[C@@H]1[C@@H](c2ccccc2)CCN1c1cc(NC2CC2)nc(N)n1] N4-Cyclopropyl-6-((2S,3S)-2- methyl-3-phenyl-pyrrolidin-1- yl)-pyrimidine-2,4-diamine | | B | B | 1.813 [310.2] |
| 56 | 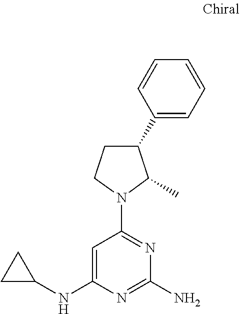 [SMILES: C[C@H]1[C@@H](c2ccccc2)CCN1c1cc(NC2CC2)nc(N)n1] N4-Cyclopropyl-6-((2R,3S)-2- methyl-3-phenyl-pyrrolidin-1- yl)-pyrimidine-2,4-diamine | | B | B | 1.840 [310.2] |
| 57 | 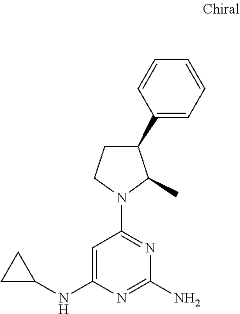 [SMILES: C[C@@H]1[C@H](c2ccccc2)CCN1c1cc(NC2CC2)nc(N)n1] N4-Cyclopropyl-6-((2R,3R)-2- methyl-3-phenyl-pyrrolidin-1- yl)-pyrimidine-2,4-diamine | | B | B | 1.844 [310.2] |
| 58 | 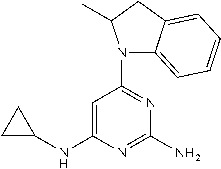 [SMILES: CC1Cc2ccccc2N1c1cc(NC2CC2)nc(N)n1] N4-Cyclopropyl-6-(2-methyl- 2,3-dihydro-indol-1-yl)- pyrimidine-2,4-diamine | 1H NMR (400 MHz, DMSO-d6) δ 8.25 (d, J = 8.0 Hz, 1H), 7.21-7.13 (m, 1H), 7.13- 7.03 (m, 1H), 6.82 (td, J = 7.4, 1.0 Hz, 1H), 6.53 (d, J = 2.6 Hz, 1H), 5.67 (s, 2H), 5.41 (s, 1H), 4.54 (ddt, J = 9.0, 6.4, 3.3 Hz, 1H), 3.42-3.33 (m, 1H), 2.62 (dd, J = 15.9, 2.0 Hz, | B | B | 1.757 [282.1]} |
| | | 1H), 1.22 (d, J = | | | |
| | | 6.2 Hz, 3H), 0.76- | | | |
| | | 0.58 (m, 2H), 0.51- | | | |
| | | 0.37 (m, 2H). | | | |
| 59 | 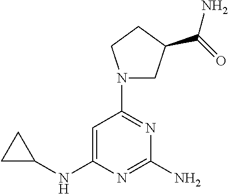 [SMILES: NC(=O)[C@@H]1CCN(c2cc(NC3CC3)nc(N)n2)C1] (R)-1-(2-Amino-6- cyclopropylamino-pyrimidin-4- yl)-pyrrolidine-3-carboxylic acid amide | 1H NMR (400 MHz, DMSO-d6) δ 7.44 (s, 1H), 6.89 (s, 1H), 6.22 (d, J = 2.5 Hz, 1H), 5.34 (s, 2H), 4.95 (s, 1H), 3.52 (m, 2H), 3.33-3.16 (m, 4H), 2.95 (m, 1H), 2.54 (s, 1H), 2.45- 2.36 (m, 1H), 2.15- 1.90 (m, 2H), 1.62 (s, 5H), 0.62 (m, 2H), 0.46- 0.34 (m, 2H). | B | | 1.087 [263.0] |
| 60 | 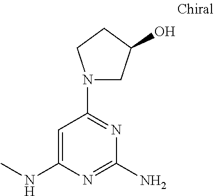 (R)-1-(2-Amino-6- methylamino-pyrimidin-4-yl)- pyrrolidin-3-ol | 1H NMR (300 MHz, DMSO-d6) δ 5.94 (d, J = 5.1 Hz, 1H), 5.35 (s, 2H), 4.86 (d, J = 3.6 Hz, 1H), 4.69 (s, 1H), 4.30 (s, 1H), 3.20 (d, J = 11.2 Hz, 2H), 2.66 (d, J = 4.9 Hz, 3H), 1.92 (m, 1H), 1.82 (m, 1H). | B | | 0.889 [210.1] |
| 61 | 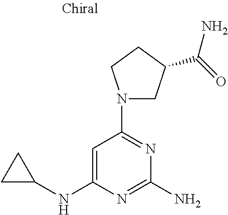 [SMILES: NC(=O)[C@H]1CCN(c2cc(NC3CC3)nc(N)n2)C1] (S)-1-(2-Amino-6- cyclopropylamino-pyrimidin-4- yl)-pyrrolidine-3-carboxylic acid amide | 1H NMR (400 MHz, DMSO-d6) δ 7.44 (s, 1H), 6.90 (s, 1H), 6.23 (d, J = 2.5 Hz, 1H), 5.34 (s, 2H), 4.96 (s, 1H), 3.54 (t, J = 9.2 Hz, 1H), 3.45 (m, 2H), 3.29-3.21 (m, 1H), 2.96 (p, J = 7.7 Hz, 1H), 2.41 (m, 1H), 2.14- 1.92 (m, 2H), 1.62 (s, 3H), 0.64 (m, 2H), 0.45-0.36 (m, 2H) | B | | 1.082 [263.2] |
| 62 | 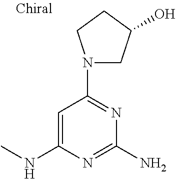 (S)-1-(2-Amino-6-methylamino- pyrimidin-4-yl)-pyrrolidin-3-ol | 1H NMR (400 MHz, DMSO-d6) δ 5.96 (d, J = 5.2 Hz, 1H), 5.38 (s, 2H), 4.85 (d, J = 3.6 Hz, 1H), 4.69 (s, 1H), 4.30 (m, 1H), 3.41- 3.34 (m, 3H), 3.20 (m, 1H), 2.67 (d, J = 4.9 Hz, 3H), 1.92 (m, 1H), 1.82 (m, 1H). | B | B | 0.896 [210.1] |
| 63 | 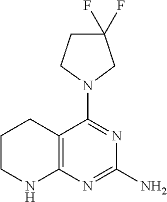 [SMILES: Nc1nc2c(c(N3CCC(F)(F)C3)n1)CCCN2] 4-(3,3-Difluoro-pyrrolidin-1-yl)- 5,6,7,8-tetrahydro-pyrido[2,3- d]pyrimidin-2-ylamine | 1H NMR (400 MHz, DMSO-d6) d 7.23 (s, 1H), 6.78 (s, 2H), 3.98 (t, J = 13.3 Hz, 2H), 3.79 (t, J = 7.3 Hz, 2H), 3.25-3.17 (m, 2H), 2.59 (t, J = 6.2 Hz, 2H), 2.47- 2.34 (m, 2H), 1.74- 1.66 (m, 2H). | A | B | 1.055 [256.3] |
| 64 | 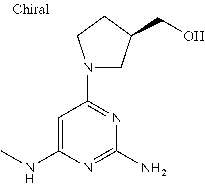 [SMILES: CNc1cc(N2CC[C@@H](CO)C2)nc(N)n1] [(R)-1-(2-Amino-6- methylamino-pyrimidin-4-yl)- pyrrolidin-3-yl]-methanol | 1H NMR (300 MHz, DMSO-d6) δ 6.00 (d, J = 5.7 Hz, 1H), 5.37 (s, 2H), 4.69 (s, 2H), 3.23 (m, 3H), 3.04 (dd, J = 10.4, 6.5 Hz, 1H), 2.66(d, J = 4.8 Hz, 3H), 2.31 (m, 1H), 2.00- 1.85 (m, 1H), 1.65 (m, 3H) | A | | 1.040 [224.1] |
| 65 | 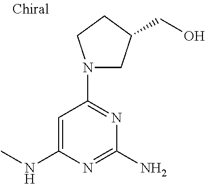 [SMILES: CNc1cc(N2CC[C@H](CO)C2)nc(N)n1] [(S)-1-(2-Amino-6- methylamino-pyrimidin-4-yl)- pyrrolidin-3-yl]-methanol | 1H NMR (300 MHz, DMSO-d6) δ 5.97 (d, J = 5.0 Hz, 1H), 5.37 (s, 2H), 4.68 (d, J = 2.7 Hz, 2H), 3.38 (s, 3H), 3.29-3.17 (m, 1H), 3.04 (dd, J = 10.4, 6.6 Hz, 1H), 2.66 (d, J = 4.8 Hz, 3H), 2.31 (m, 1H), 1.93 (m, 1H), 1.73- 1.56 (m, 1H). | A | | 1.030 [224.2] |
| 66 | 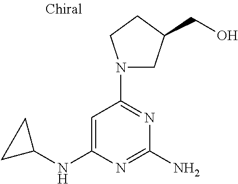 [SMILES: Nc1nc(NC2CC2)cc(N2CC[C@@H](CO)C2)n1] [(R)-1-(2-Amino-6- cyclopropylamino-pyrimidin-4- yl)-pyrrolidin-3-yl]-methanol | 1H NMR (400 MHz, DMSO-d6) δ 6.19 (d, J = 2.4 Hz, 1H), 5.30 (s, 2H), 4.94 (s, 1H), 4.65 (t, J = 5.2 Hz, 1H), 3.49-3.34 (m, 4H), 3.06 (dd, J = 10.4, 6.7 Hz, 1H), 2.39 (m, 1H), 2.32 (m, 1H), 1.99- 1.86(m, 1H), 1.65 (m, 1H), 0.62 (m, 2H), 0.42-0.33 (m, 2H). | A | | 1.144 [250.2] |
| 67 | 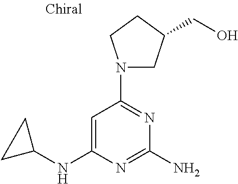 [SMILES: Nc1nc(NC2CC2)cc(N2CC[C@H](CO)C2)n1] [(S)-1-(2-Amino-6- cyclopropylamino-pyrimidin-4- yl)-pyrrolidin-3-yl]-methanol | 1H NMR (400 MHz, DMSO-d6) δ 6.20 (d, J = 2.4 Hz, 1H), 5.31 (s, 2H), 4.94 (s, 1H), 4.66 (t, J = 5.2 Hz, 1H), 3.39 (td, J = 11.7, 11.0, 5.8 Hz, 4H), 3.25 (m, 1H), 3.06 (dd, J = 10.3, 6.6 Hz, 1H), 2.39 (m, 1H), 2.32 (m, 1H), 2.01-1.85 (m, 1H), 1.74-1.58 (m, 1H), 0.70- 0.58 (m, 2H), 0.41- | B | | 1.151 [250.1] |
| | | 0.36 (m, 2H). | | | |
| 68 | 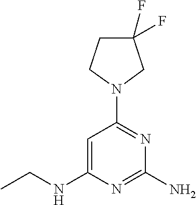 [SMILES: CCNc1cc(N2CCC(F)(F)C2)nc(N)n1] 6-(3,3-Difluoro-pyrrolidin-1-yl)- N4-ethyl-pyrimidine-2,4- diamine | 1H NMR (300 MHz, DMSO-d6) δ 6.17 (t, J = 5.6 Hz, 1H), 5.53 (s, 2H), 4.79 (s, 1H), 3.70 (t, J = 13.5 Hz, 3H), 3.49 (t, J = 7.3 Hz, 2H), 3.16 (qd, J = 7.2, 5.5 Hz, 2H), 2.43 (m, 1H), 1.07 (t, J = 7.2 Hz, 3H). | B | | 1.319 [244.1] |
| 69 | 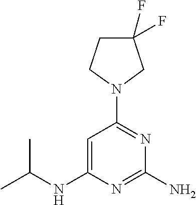 [SMILES: CC(C)Nc1cc(N2CCC(F)(F)C2)nc(N)n1] 6-(3,3-Difluoro-pyrrolidin-1-yl)- N4-isopropyl-pyrimidine-2,4- diamine | 1H NMR (300 MHz, DMSO-d6) δ 6.08 (d, J = 8.2 Hz, 1H), 5.56 (s, 2H), 4.80 (s, 1H), 3.70 (t, J = 13.4 Hz, 3H), 3.48 (t, J = 7.3 Hz, 3H), 2.43 (m, 1H), 1.08 (d, J = 6.4 Hz, 6H). | B | | 1.417 [258.1] |
| 70 | 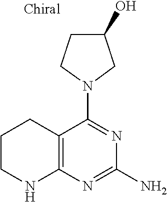 [SMILES: Nc1nc2c(c(N3CC[C@@H](O)C3)n1)CCCN2] (R)-1-(2-Amino-5,6,7,8- tetrahydro-pyrido[2,3- d]pyrimidin-4-yl)-pyrrolidin-3-ol | | A | | 0.880 [236.2] |
| 71 | 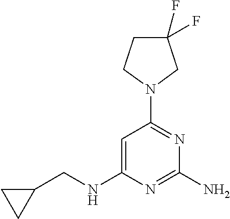 [SMILES: Nc1nc(NCC2CC2)cc(N2CCC(F)(F)C2)n1] N4-Cyclopropylmethyl-6-(3,3- difluoro-pyrrolidin-1-yl)- pyrimidine-2,4-diamine | | B | | 1.453 [270.2] |
| 72 | 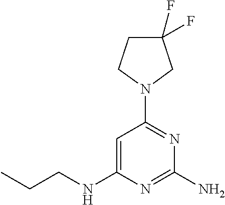 [SMILES: CCCNc1cc(N2CCC(F)(F)C2)nc(N)n1] 6-(3,3-Difluoro-pyrrolidin-1-yl)- N4-propyl-pyrimidine-2,4- diamine | 1H NMR (300 MHz, DMSO-d6) δ 8.15 (s, 1H), 6.24 (t, J = 5.7 Hz, 1H), 4.80 (s, 1H), 3.70 (t, J = 13.5 Hz, 2H), 3.48 (t, J = 7.3 Hz, 2H), 3.09 (q, J = 6.6 Hz, 2H), 2.42 (dt, J = 14.4, 7.2 Hz, 2H), 1.48 (h, J = 7.3 Hz, 2H), 0.87 (t, J = 7.4 Hz, 3H). | B | | 1.460 [258.1] |
| 73 | 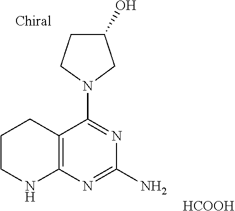 [SMILES: Nc1nc2c(c(N3CC[C@H](O)C3)n1)CCCN2 ; "HCOOH"] (S)-1-(2-Amino-5,6,7,8- tetrahydro-pyrido[2,3- d]pyrimidin-4-yl)-pyrrolidin-3-ol- formiate | 1H NMR (400 MHz, DMSO-d6) ⊐ 7.11-6.35 (m, 1H), 6.03-5.59 (m, 2H), 5.08- 4.63 (m, 1H), 4.27- 4.21 (m, 1H), 3.66-3.04 (m, 6H), 2.58-2.44 (m, 2H), 1.89- 1.78 (m, 1H), 1.78- 1.69 (m, 2H), 1.63-1.51 (m, 1H). | B | | 0.878 [236.2] |
| 74 | 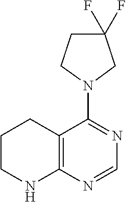 [SMILES: FC1(F)CCN(c2ncnc3c2CCCN3)C1] 4-(3,3-Difluoro-pyrrolidin-1-yl)- 5,6,7,8-tetrahydro-pyrido[2,3- d]pyrimidine | 1H NMR (300 MHz, DMSO-d6) ⊐ 8.26-8.09 (m, 2H), 4.12 (t, J = 13.1 Hz, 2H), 3.91 (t, J = 7.4 Hz, 2H), 3.33-3.24 (m, 2H), 2.77 (t, J = 6.2 Hz, 2H), 2.54- 2.37 (m, 2H), 1.81- 1.69 (m, 2H). | C | | 0.991 [241.1] |
| 75 | 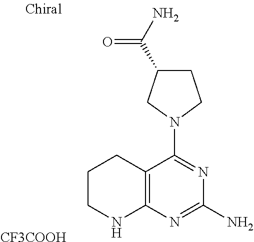 [SMILES: "CF3COOH" ; NC(=O)[C@@H]1CCN(c2nc(N)nc3c2CCCN3)C1] (R)-1-(2-Amino-5,6,7,8- tetrahydro-pyrido[2,3- d]pyrimidin-4-yl)-pyrrolidine-3- carboxylic acid amide- trifluoroacetate | 1H NMR (400 MHz, DMSO-d6) ⊐ 11.59-10.04 (m, 1H), 7.56-6.89 (m, 4H), 3.78- 3.58 (m, 4H), 3.31- 3.22 (m, 1H), 3.22-3.13 (m, 1H), 2.99-2.87 (m, 1H), 2.71- 2.58 (m, 2H), 2.15- 2.02 (m, 1H), 2.00-1.88 (m, 1H), 1.83-1.72 (m, 1H), 1.71- 1.57 (m, 1H). | C | | 0.865 [263.1] |
| 76 | 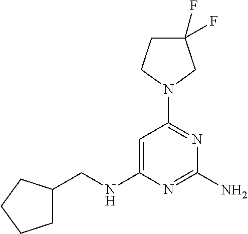 [SMILES: Nc1nc(NCC2CCCC2)cc(N2CCC(F)(F)C2)n1] N4-Cyclopentylmethyl-6-(3,3- difluoro-pyrrolidin-1-yl)- pyrimidine-2,4-diamine | 1H NMR (300 MHz, DMSO-d6) δ 6.26 (t, J = 5.6 Hz, 1H), 5.56 (s, 2H), 4.83 (s, 1H), 3.71 (t, J = 13.4 Hz, 2H), 3.49 (t, J = 7.3 Hz, 2H), 3.07 (dd, J = 7.3, 5.6 Hz, 3H), 2.43 (dt, J = 14.4, 7.2 Hz, 2H), 2.06 (h, J = 7.4 Hz, 1H), 1.79-1.63 (m, 2H), 1.63-1.42 (m, 3H), 1.31- 1.14 (m, 2H). | B | B | 1.733 [298.2] |
| 77 | 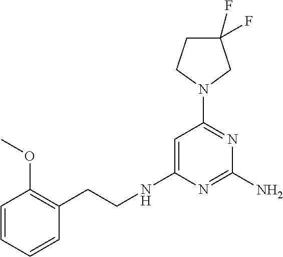 [SMILES: COc1ccccc1CCNc1cc(N2CCC(F)(F)C2)nc(N)n1] 6-(3,3-Difluoro-pyrrolidin-1-yl)- N4-[2-(2-methoxy-phenyl)- ethyl]-pyrimidine-2,4-diamine | 1H NMR (300 MHz, DMSO-d6) δ 7.24-7.14 (m, 2H), 6.96 (dd, J = 8.2, 1.1 Hz, 1H), 6.87 (td, J = 7.4, 1.1 Hz, 1H), 6.21 (s, 1H), 5.53 (s, 2H), 4.82 (s, 1H), 3.79 (s, 3H), 3.68 (m, 2H), 3.48 (m, 3H), 3.29 (m, 3H), 2.77 (dd, J = 8.7, 6.5 Hz, 2H), 2.43 (m, 2H). | B | B | 1.789 [350.1] |
| 78 | 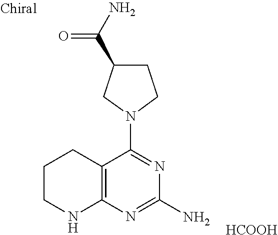 [SMILES: NC(=O)[C@H]1CCN(c2nc(N)nc3c2CCCN3)C1 ; "HCOOH"] (S)-1-(2-Amino-5,6,7,8- tetrahydro-pyrido[2,3- d]pyrimidin-4-yl)-pyrrolidine-3- carboxylic acid amide- formiate | 1H NMR (300 MHz, DMSO-d6) ⊐ 7.42 (s, 1H), 6.91 (s, 1H), 6.40-6.34 (m, 1H), 5.59 (s, 2H), 3.64-3.47 (m, 4H), 3.23- 3.04 (m, 2H), 2.91- 2.78 (m, 1H), 2.58-2.49 (m, 2H), 2.05-1.81 (m, 2H), 1.79- 1.66 (m, 1H), 1.65- 1.48 (m, 1H). | A | B | 0.873 [263.2] |
| 79 | 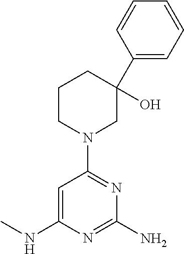 [SMILES: CNc1cc(N2CCCC(O)(c3ccccc3)C2)nc(N)n1] 1-(2-Amino-6-methylamino- pyrimidin-4-yl)-3-phenyl- piperidin-3-ol | 1H NMR (400 MHz, DMSO-d6) δ 7.64-7.49 (m, 2H), 7.33 (dd, J = 8.4, 6.9 Hz, 2H), 7.28-7.18 (m, 1H), 6.00 (d, J = 5.3 Hz, 1H), 5.43 (s, 2H), 5.00 (s, 1H), 4.88 (s, 1H), 4.00 (m, 1H), 3.81 (d, J = 13.1 Hz, 1H), 3.23 (d, J = 13.1 Hz, 1H), 2.97 (ddd, J = 13.1, 10.8, 3.1 Hz, 1H), 2.67 (d, J = 4.8 Hz, 3H), 2.06-1.93 (m, 1H), 1.95- 1.79 (m, 1H), 1.72 | B | | 1.427 [300.2] |
| | | (m, 1H), 1.45 (m, | | | |
| | | 1H). | | | |
| 80 | 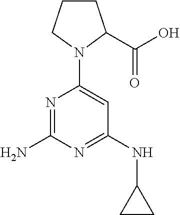 [SMILES: Nc1nc(NC2CC2)cc(N2CCCC2C(=O)O)n1] 1-(2-Amino-6- cyclopropylamino-pyrimidin-4- yl)-pyrrolidine-2-carboxylic acid | 1H NMR (400 MHz, DMSO-d6) ⊐ 11.28-10.86(m, 1H), 7.99 (s, 1H), 7.53-7.15 (m, 2H), 5.23-4.94 (m, 1H), 4.62- 4.27 (m, 1H), 2.61- 2.50 (m, 1H), 2.34-2.16 (m, 1H), 2.12-1.84 (m, 3H), 1.70- 1.49 (m, 1H), 0.87- 0.73 (m, 2H), 0.60-0.44 (m, 2H). | C | | 0.972 [264.3] |
| 81 | 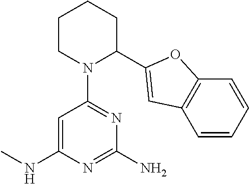 [SMILES: CNc1cc(N2CCCCC2c2cc3ccccc3o2)nc(N)n1] 6-(2-Benzofuran-2-yl-piperidin- 1-yl)-N4-methyl-pyrimidine-2,4- diamine | 1H NMR (400 MHz, DMSO-d6) δ 7.60-7.45 (m, 2H), 7.20 (m, 2H), 6.49 (s, 1H), 6.11 (d, J = 5.1 Hz, 1H), 5.97 (s, 1H), 5.48 (s, 2H), 5.08 (s, 1H), 4.01 (m, 1H), 2.92 (t, J = 12.4 Hz, 1H), 2.68 (d, J = 4.9 Hz, 3H), 2.28 (d, J = 13.6 Hz, 1H), 1.80 (m, 1H), 1.73-1.60 (m, 2H), 1.44 (m, 2H). | A | B | 1.830 [324.2] |
| 82 | 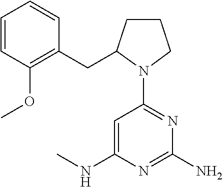 [SMILES: CNc1cc(N2CCCC2Cc2ccccc2OC)nc(N)n1] 6-[2-(2-Methoxy-benzyl)- pyrrolidin-1-yl]-N4-methyl- pyrimidine-2,4-diamine | 1H NMR (300 MHz, DMSO-d6) δ 7.20 (m, 2H), 6.97 (dd, J = 8.6, 1.1 Hz, 1H), 6.89 (td, J = 7.4, 1.1 Hz, 1H), 5.96 (d, J = 5.1 Hz, 1H), 5.38 (s, 2H), 4.95 (s, 1H), 4.06 (s, 1H), 3.82 (s, 3H), 3.29 (m, 2H), 3.04 (dd, J = 12.7, 3.4 Hz, 1H), 2.71 (d, J = 4.9 Hz, 3H), 1.86 (m, 2H), 1.66 (m, 2H). | A | | 1.705 [314.2] |
| 83 | 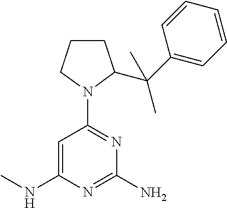 [SMILES: CNc1cc(N2CCCC2C(C)(C)c2ccccc2)nc(N)n1] N4-Methyl-6-[2-(1-methyl-1- phenylethyl)-pyrrolidin-1-yl]- pyrimidine-2,4-diamine | 1H NMR (400 MHz, DMSO-d6) δ 7.48-7.40 (m, 2H), 7.34-7.26 (m, 2H), 7.24- 7.14 (m, 1H), 6.01 (d, J = 5.1 Hz, 1H), 5.41 (s, 2H), 4.92 (s, 1H), 4.60 (s, 1H), 3.12 (m, 1H), 2.69 (d, J = 4.9 Hz, 3H), 1.53 (m, 3H), 1.31 (s, 3H), 1.27 (s, 3H), 1.07 (M, 1H). | A | | 1.817 [312.2][ |
| 84 | 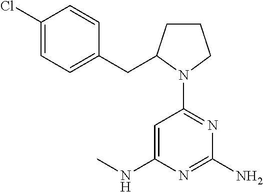 [SMILES: CNc1cc(N2CCCC2Cc2ccc(Cl)cc2)nc(N)n1] 6-[2-(4-Chloro-benzyl)- pyrrolidin-1-yl]-N4-methyl- pyrimidine-2,4-diamine | 1H NMR (300 MHz, DMSO-d6) δ 7.43-7.20 (m, 4H), 6.82 (s, 1H), 6.47 (s, 2H), 4.90 (s, 1H), 4.18 (s, 1H), 3.30 (m, 2H), 3.01 (dd, J = 13.1, 3.3 Hz, 1H), 2.76 (d, J = 4.8 Hz, 3H), 2.61 (dd, J = 13.0, 9.3 Hz, 1H), 1.94- 1.64 (m, 4H). | A | B | 1.808 [318.1] |
| 85 | 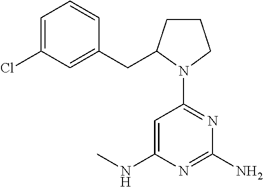 [SMILES: CNc1cc(N2CCCC2Cc2cccc(Cl)c2)nc(N)n1] 6-[2-(3-Chloro-benzyl)- pyrrolidin-1-yl]-N4-methyl- pyrimidine-2,4-diamine | 1H NMR (400 MHz, DMSO-d6) δ 7.40-7.18 (m, 4H), 6.27 (s, 1H), 5.74 (s, 2H), 4.83 (s, 1H), 4.14 (s, 1H), 3.57 (s, 2H), 3.11-3.01 (m, 1H), 2.71 (d, J = 4.9 Hz, 3H), 2.58 (dd, J = 12.9, 9.4 Hz, 1H), 1.88- 1.60 (m, 4H). | A | A | 1.766 [318.1] |
| 86 | 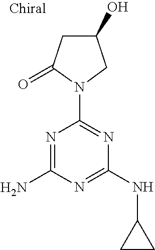 [SMILES: Nc1nc(NC2CC2)nc(N2C[C@H](O)CC2=O)n1] (R)-1-(2-Amino-6- cyclopropylamino-pyrimidin-4- yl)-4-hydroxy-pyrrolidin-2-one | 1H NMR (300 MHz, DMSO-d6) d 8.90-8.11 (m, 1H), 8.11-6.38 (m, 2H), 5.76- 4.98 (m, 1H), 4.42- 4.33 (m, 1H), 3.94 (dd, J = 11.4, 4.8 Hz, 1H), 3.86- 3.74 (m, 1H), 2.95 (dd, J = 17.4, 5.8 Hz, 1H), 2.76- 2.59 (m, 1H), 2.38 (d, J = 17.5 Hz, 1H), 0.85-0.77 (m, 2H), 0.62- 0.51 (m, 2H). | C | C | 0.817 [250.3] |
| 87 | 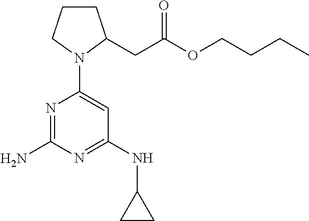 [SMILES: CCCCOC(=O)CC1CCCN1c1cc(NC2CC2)nc(N)n1] [1-(2-Amino-6- cyclopropylamino-pyrimidin-4- yl)-pyrrolidin-2-yl]-acetic acid butyl ester | 1H NMR (500 MHz, DMSO-d6) δ 10.89 (s, 1H), 7.90 (s, 1H), 7.31 (s, 2H), 5.12 (s, 1H), 4.48 (s, 1H), 4.04 (t, J = 6.7 Hz, 2H), 3.43 (s, 2H), 2.86 (s, 1H), 1.99 (d, J = 43.2 Hz, 3H), 1.81 (s, 1H), 1.56 (dq, J = 8.5, 6.7 Hz, 2H), 1.42-1.26 (m, 2H), 0.90 (t, J = 7.4 Hz, 3H), 0.82 (s, 2H), 0.65-0.42 (m, 2H) | B | B | 1.363 [334.3] |
| 88 | 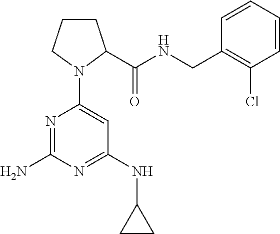 [SMILES: Nc1nc(NC2CC2)cc(N2CCCC2C(=O)NCc2ccccc2Cl)n1] 1-(2-Amino-6- cyclopropylamino-pyrimidin-4- yl)-pyrrolidine-2-carboxylic acid 2-chloro-benzylamide | 1H NMR (400 MHz, DMSO-d6) d 11.01 (s, 1H), 8.80- 8.27 (m, 1H), 8.09-7.83 (m, 1H), 7.55-7.20 (m, 6H), 5.26- 4.91 (m, 1H), 4.71- 4.14 (m, 3H), 3.77-3.47 (m, 2H), 2.59-2.11 (m, 1H), 2.07- 1.76 (m, 3H), 0.90- 0.66 (m, 2H), 0.61-0.39 (m, 2H). | C | C | 1.209 [387.2] |
| 89 | 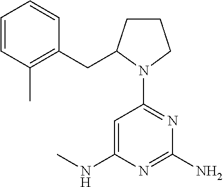 [SMILES: CNc1cc(N2CCCC2Cc2ccccc2C)nc(N)n1] N4-Methyl-6-[2-(2-methyl- benzyl)-pyrrolidin-1-yl]- pyrimidine-2,4-diamine | 1H NMR (400 MHz, DMSO-d6) δ 7.22 (dd, J = 6.9, 2.0 Hz, 1H), 7.18- 7.07 (m, 3H), 6.03 (d, J = 5.2 Hz, 1H), 5.30 (s, 2H), 4.79 (s, 1H), 4.21 (s, 1H), 3.37 (m, 1H), 3.21 (m, 1H), 3.13 (dd, J = 13.2, 3.5 Hz, 1H), 2.68 (d, J = 4.9 Hz, 3H), 2.48- 2.43 (m, 1H), 2.42 (s, 3H), 1.97 (m, 1H), 1.86 (m, 1H), 1.66 (m, 2H). | A | | 1.811 [298.2] |
| 90 | 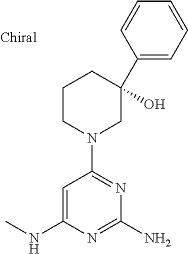 [SMILES: CNc1cc(N2CCC[C@@](O)(c3ccccc3)C2)nc(N)n1] (R)-1-(2-Amino-6- methylamino-pyrimidin-4-yl)-3- phenyl-piperidin-3-ol | — | B | | 1.497 [300.1] |
| 91 | 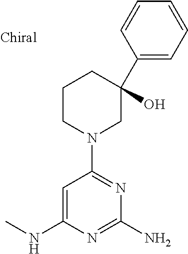 [SMILES: CNc1cc(N2CCC[C@](O)(c3ccccc3)C2)nc(N)n1] (S)-1-(2-Amino-6-methylamino- pyrimidin-4-yl)-3-phenyl- piperidin-3-ol | — | B | B | 1.504 [300.2] |
| 92 | 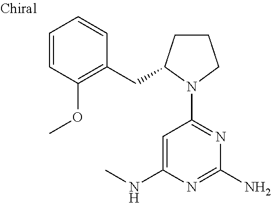 [SMILES: CNc1cc(N2CCC[C@H]2Cc2ccccc2OC)nc(N)n1] 6-[(S)-2-(2-Methoxy-benzyl)- pyrrolidin-1-yl]-N4-methyl- pyrimidine-2,4-diamine | 1H NMR (300 MHz, DMSO-d6) δ 7.21 (m, 2H), 7.02- 6.94 (m, 1H), 6.89 (m, 1H), 5.94 (d, J = 5.0 Hz, 1H), 5.36 (s, 2H), 4.95 (s, 1H), 4.06 (br. s, 1H), 3.83 (s, 3H), 3.37 (m, 1H), 3.05 (dd, J = 12.9, 3.5 Hz, 1H), 2.72 (d, J = 4.9 Hz, 3H), 1.87 (m, 2H), 1.73- 1.59 (m, 2H). | A | | 1.336 [314.2] |
| 93 | 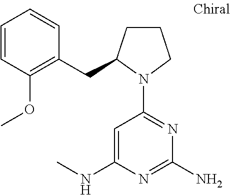 [SMILES: CNc1cc(N2CCC[C@@H]2Cc2ccccc2OC)nc(N)n1] 6-[(R)-2-(2-Methoxy-benzyl)- pyrrolidin-1-yl]-N4-methyl- pyrimidine-2,4-diamine | 1H NMR (300 MHz, DMSO-d6) δ 7.21 (m, 2H), 7.03- 6.95 (m, 1H), 6.90 (m, 1H), 5.94 (d, J = 5.0 Hz, 1H), 5.36 (s, 2H), 4.95 (s, 1H), 4.07 (br. S, 1H), 3.83 (s, 3H), 3.38 (m, 1H), 3.05 (dd, J = 12.9, 3.5 Hz, 1H), 2.72 (d, J = 4.8 Hz, 3H), 1.98- 1.74 (m, 2H), 1.65 (m, 2H). | A | | 1.335 [314.2] |
| 94 | 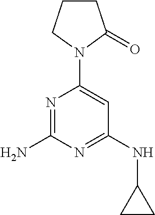 [SMILES: Nc1nc(NC2CC2)cc(N2CCCC2=O)n1] 1-(2-Amino-6- cyclopropylamino-pyrimidin-4- yl)-pyrrolidin-2-one | 1H NMR (400 MHz, DMSO-d6) d 11.28-10.86 (m, 1H), 7.99 (s, 1H), 7.53-7.15 (m, 2H), 5.23-4.94 (m, 1H), 4.62- 4.27 (m, 1H), 2.61- 2.50 (m, 1H), 2.34-2.16 (m, 1H), 2.12-1.84 (m, 3H), 1.70- 1.49 (m, 1H), 0.87- 0.73 (m, 2H), 0.60-0.44 (m, 2H). | C | C | 0.972 [264.3] |
| 95 | 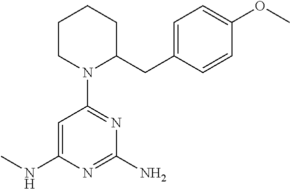 6-[2-(4-Methoxy-benzyl)- piperidin-1-yl]-N4-methyl- pyrimidine-2,4-diamine | 1H NMR (300 MHz, DMSO-d6) δ 7.19 (d, J = 8.6 Hz, 2H), 6.90-6.80 (m, 2H), 6.03 (d, J = 5.2 Hz, 1H), 5.42 (s, 2H), 4.99 (s, 1H), 4.44 (br. s, 1H), 4.10 (br. s, 1H), 3.72 (s, 3H), 2.83 (dd, J = 13.3, 10.4 Hz, 2H), 2.68 (d, J = 4.9 Hz, 3H), 2.59 (dd, J = 13.1, 5.0 Hz, 1H), 1.78- 1.61 (m, 3H), 1.60- | A | | 1.766 [328.2] |
| | | 1.12 (m, 6H). | | | |
| 96 | 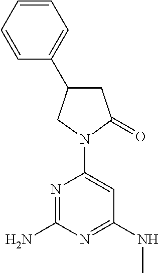 [SMILES: CNc1cc(N2CC(c3ccccc3)CC2=O)nc(N)n1] 1-(2-Amino-6-methylamino- pyrimidin-4-yl)-4-phenyl- pyrrolidin-2-one | 1H NMR (300 MHz, DMSO-d6) d 8.55-7.54 (m, 2H), 7.43-7.22 (m, 7H), 4.34- 4.17 (m, 1H), 3.82- 3.58 (m, 2H), 3.06-2.94 (m, 1H), 2.92-2.81 (m, 4H). | C | | 1.130 [284.1] |
| 97 | 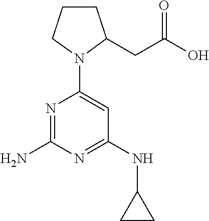 [SMILES: Nc1nc(NC2CC2)cc(N2CCCC2CC(=O)O)n1] [1-(2-Amino-6- cyclopropylamino-pyrimidin-4- yl)-pyrrolidin-2-yl]acetic acid | 1H NMR (400 MHz, DMSO-d6) d 11.51-11.05 (m, 1H), 7.97 (s, 1H), 7.36 (s, 2H), 5.12 (s, 1H), 4.67-4.20 (m, 1H), 3.57- 3.21 (m, 2H), 2.95- 2.60 (m, 1H), 2.59-2.52 (m, 1H), 2.35 (dd, J = 16.0, 10.3 Hz, 1H), 2.11-1.76 (m, 4H), 0.85-0.72 (m, 2H), 0.58- 0.48 (m, 2H). | B | C | 1.006 [278.2] |
| 98 | 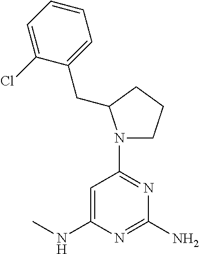 [SMILES: CNc1cc(N2CCCC2Cc2ccccc2Cl)nc(N)n1] 6-[2-(2-Chloro-benzyl)- pyrrolidin-1-yl]-N4-methyl- pyrimidine-2,4-diamine | 1H NMR (300 MHz, DMSO-d6) d 11.61-11.25 (m, 1H), 7.60-7.13 (m, 7H), 5.12- 4.88 (m, 1H), 4.60- 4.11 (m, 1H), 3.61-3.20 (m, 1H), 3.12 (dd, J = 13.6, 4.6 Hz, 1H), 2.90-2.68 (m, 4H), 2.12-1.65 (m, 4H). | A | B | 1.360 [318.2] |
| 99 | 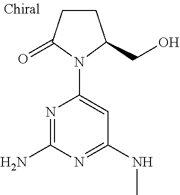 [SMILES: CNc1cc(N2C(=O)CC[C@H]2CO)nc(N)n1] (S)-1-(2-Amino-6-methylamino- pyrimidin-4-yl)-5- hydroxymethyl-pyrrolidin-2-one | 1H NMR (300 MHz, DMSO-d6) d 8.51-7.98 (m, 1H), 7.87-7.14 (m, 2H), 6.58- 5.90 (m, 1H), 4.51- 4.33 (m, 1H), 3.77-3.67 (m, 1H), 3.56-3.47 (m, 1H), 2.90- 2.81 (m, 3H), 2.80- 2.67 (m, 1H), 2.47-2.37 (m, 1H), 2.29-1.94 (m, 2H). | C | | 0.799 [238.1] |
| 100 | 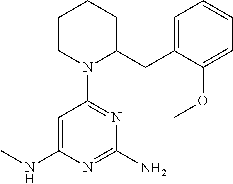 [SMILES: CNc1cc(N2CCCCC2Cc2ccccc2OC)nc(N)n1] 6-[2-(2-Methoxy-benzyl)- piperidin-1-yl]-N4-methyl- pyrimidine-2,4-diamine | 1H NMR (500 MHz, DMSO-d6) δ 7.21 (dd, J = 7.4, 1.7 Hz, 1H), 7.17 (td, J = 7.8, 1.8 Hz, 1H), 6.94 (dd, J = 8.3, 1.0 Hz, 1H), 6.86 (td, J = 7.4, 1.1 Hz, 1H), 5.95 (q, J = 4.9 Hz, 1H), 5.35 (s, 2H), 4.99 (s, 1H), 4.37 (br. m, 2H), 3.81 (s, 3H), 3.00 (dd, J = 12.9, 10.0 Hz, 1H), 2.84 (td, J = 13.1, 2.9 | A | B | 1.860 [328.3] |
| | | Hz, 1H), 2.68 (d, J = | | | |
| | | 4.9 Hz, 3H), 2.62 | | | |
| | | (dd, J = 12.9, 5.5 | | | |
| | | Hz, 1H), 1.78 (qt, J = | | | |
| | | 12.9, 4.2 Hz, | | | |
| | | 1H), 1.70-1.62 | | | |
| | | (m, 1H), 1.60 (s, | | | |
| | | 1H), 1.52 (m, 1H), | | | |
| | | 1.41-1.21 (m, | | | |
| | | 4H). | | | |
| 101 | 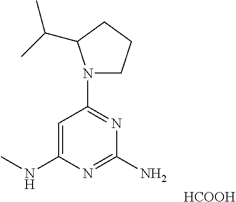 [SMILES: CNc1cc(N2CCCC2C(C)C)nc(N)n1 ; "HCOOH"] 6-(2-Isopropyl-pyrrolidin-1-yl)- N4-methyl-pyrimidine-2,4- diamine-formate | 1H NMR (400 MHz, DMSO-d6) d 11.22-10.88 (m, 1H), 7.08-6.97 (m, 1H), 6.79- 6.63 (m, 2H), 4.91 (s, 1H), 3.98-3.89 (m, 1H), 3.49- 3.32 (m, 2H), 2.76 (d, J = 4.8 Hz, 3H), 2.27-2.16 (m, 1H), 1.96-1.77 (m, 4H), 0.87 (d, J = 6.9 Hz, 3H), 0.76 (d, J = 6.8 Hz, 3H). | A | | 1.210 [236.2] |
| 102 | 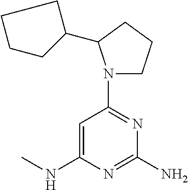 [SMILES: CNc1cc(N2CCCC2C2CCCC2)nc(N)n1] 6-(2-Cyclopentyl-pyrrolidin-1- yl)-N4-methyl-pyrimidine-2,4- diamine | 1H NMR (400 MHz, DMSO-d6) d 11.25-10.66 (m, 1H), 7.78-6.99 (m, 3H), 5.12- 4.85 (m, 1H), 4.48- 3.87 (m, 1H), 3.54-3.35 (m, 2H), 2.79 (d, J = 4.8 Hz, 3H), 2.25- 1.73 (m, 5H), 1.68- 1.12 (m, 8H). | A | | 1.313 [262.2] |
| 103 | 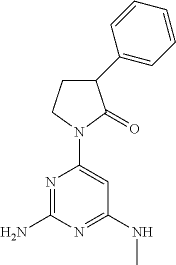 [SMILES: CNc1cc(N2CCC(c3ccccc3)C2=O)nc(N)n1] 1-(2-Amino-6-methylamino- pyrimidin-4-yl)-3-phenyl- pyrrolidin-2-one | 1H NMR (500 MHz, DMSO-d6) d 7.38-7.20 (m, 6H), 6.77-6.64 (m, 1H), 5.86 (s, 2H), 4.11-4.03 (m, 1H), 3.93 (t, J = 9.3 Hz, 1H), 3.83- 3.76 (m, 1H), 2.71 (d, J = 4.7 Hz, 3H), 2.49-2.41 (m, 1H), 2.14-2.02 (m, 1H). | B | | 1.123 [284.2] |
| 104 | 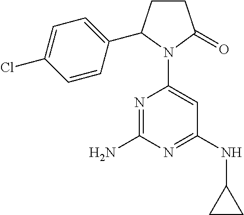 [SMILES: Nc1nc(NC2CC2)cc(N2C(=O)CCC2c2ccc(Cl)cc2)n1] 1-(2-Amino-6- cyclopropylamino-pyrimidin-4- yl)-5-(4-chloro-phenyl)- pyrrolidin-2-one | 1H NMR (300 MHz, DMSO-d6) d 7.38-7.32 (m, 2H), 7.26-7.20 (m, 2H), 6.95- 6.84 (m, 2H), 5.71- 5.61 (m, 3H), 2.75-2.40 (m, 4H), 1.86-1.77 (m, 1H), 0.67- 0.60 (m, 2H), 0.44- 0.37 (m, 2H). | C | | |
| 105 | 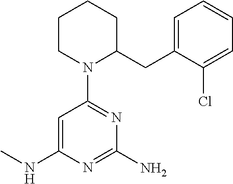 [SMILES: CNc1cc(N2CCCCC2Cc2ccccc2Cl)nc(N)n1] 6-[2-(2-Chloro-benzyl)- piperidin-1-yl]-N4-methyl- pyrimidine-2,4-diamine | 1H NMR (300 MHz, DMSO-d6) δ 7.42 (m, 2H), 7.32- 7.16 (m, 2H), 6.00 (br. d, J = 5.4 Hz, 1H), 5.38 (br. s, 2H), 4.97 (br. s, 1H), 4.60 (br. s, 1H), 4.23 (br. d, J = 13.1 Hz, 1H), 3.21- 3.03 (m, 3H), 2.99-2.78 (m, 2H), 2.65 (d, J = 4.8 Hz, 3H), 1.48- 1.14 (m, 4H). | A | B | 1.923 [332.1] |
| 106 | 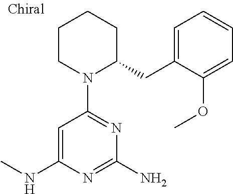 [SMILES: CNc1cc(N2CCCC[C@@H]2Cc2ccccc2OC)nc(N)n1] 6-[(R)-2-(2-Methoxy-benzyl)- piperidin-1-yl]-N4-methyl- pyrimidine-2,4-diamine | — | A | | 1.803 [328.2] |
| 107 | 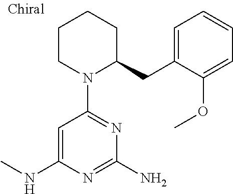 [SMILES: CNc1cc(N2CCCC[C@H]2Cc2ccccc2OC)nc(N)n1] 6-[(S)-2-(2-Methoxy-benzyl)- piperidin-1-yl]-N4-methyl- pyrimidine-2,4-diamine | — | B | B | 1.821 [328.2] |
| 108 | 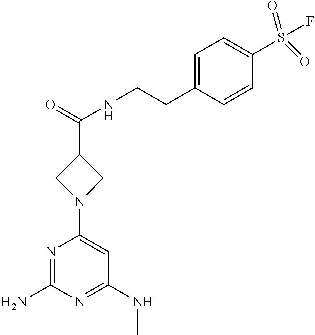 [SMILES: CNc1cc(N2CC(C(=O)NCCc3ccc(S(=O)(=O)F)cc3)C2)nc(N)n1] 4-(2-{[1-(2-Amino-6- methylamino-pyrimidin-4-yl)- azetidine-3-carbonyl]-amino}- ethyl)-benzenesulfonyl fluoride | 1H NMR (400 MHz, DMSO-d6) δ 8.18 (t, J = 5.8 Hz, 1H), 8.06 (d, J = 8.5 Hz, 2H), 7.63 (d, J = 8.2 Hz, 2H), 7.34 (m, 2H), 4.80 (s, 1H), 4.17-4.09 (m, 2H), 3.97 (s, 2H), 3.42 (m, 4H), 2.93 (m, 2H), 2.83- 2.73 (m, 3H). | A | | |
| 109 | 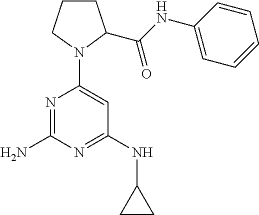 [SMILES: Nc1nc(NC2CC2)cc(N2CCCC2C(=O)Nc2ccccc2)n1] 1-(2-Amino-6- cyclopropylamino-pyrimidin-4- yl)-pyrrolidine-2-carboxylic acid phenylamide | 1H NMR (400 MHz, DMSO-d6) d 10.99-10.79 (m, 1H), 10.39-9.90 (m, 1H), 8.00- 7.84 (m, 1 H), 7.58 (d, J = 7.9 Hz, 2H), 7.50-7.00 (m, 5H), 5.29-4.95 (m, 1 H), 4.80- 4.37 (m, 1H), 3.81- 3.38 (m, 1H), 2.69-2.18 (m, 2H), 2.14-1.90 (m, 3H), 0.90- 0.23 (m, 4H). | | | |
| 110 | 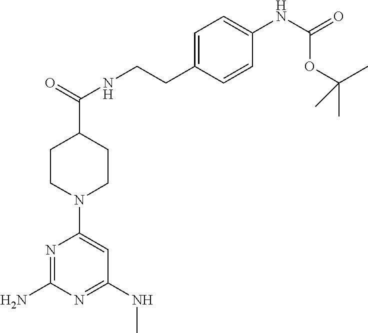 [SMILES: CNc1cc(N2CCC(C(=O)NCCc3ccc(NC(=O)OC(C)(C)C)cc3)CC2)nc(N)n1] [4-(2-{[1-(2-Amino-6- methylamino-pyrimidin-4-yl)- piperidine-4-carbonyl]-amino}- ethyl)-phenyl]-carbamic acid tert-butyl ester | | | | 1.726 [470.3] |
| 111 | 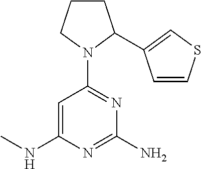 [SMILES: CNc1cc(N2CCCC2c2ccsc2)nc(N)n1] N4-Methyl-6-(2-thiophen-3-yl- pyrrolidin-1-yl)-pyrimidine-2,4- diamine | 1H NMR (400 MHz, DMSO-d6, 90° C.) d 7.46 (dd, J = 5.0, 3.0 Hz, 1H), 7.31-7.18 (m, 2H), 7.07-6.88 (m, 3H), 5.24- 5.14 (m, 1H), 4.89 (s, 1H), 3.77-3.68 (m, 1H), 3.64- 3.54 (m, 1H), 2.73 (s, 3H), 2.37-2.23 (m, 1H), 2.06- 1.91 (m, 3H). | A | | 1.182 [276.1] |
| 112 | 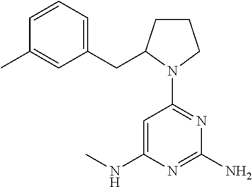 [SMILES: CNc1cc(N2CCCC2Cc2cccc(C)c2)nc(N)n1] N4-Methyl-6-[2-(3-methyl- benzyl)-pyrrolidin-1-yl]- pyrimidine-2,4-diamine | 1H NMR (300 MHz, DMSO-d6) d 11.35-11.11 (m, 1H), 7.52-7.25 (m, 3H), 7.24- 7.15 (m, 1H), 7.13- 7.01 (m, 3H), 5.09-4.87 (m, 1H), 4.45-3.99 (m, 1H), 3.69- 3.20 (m, 1H), 3.17- 2.84 (m, 1H), 2.81 (d, J = 4.7 Hz, 3H), 2.64-2.44 (m, 2H), 2.30 (s, 3H), 1.99-1.64 | A | B | 1.355 [298.2] |
| | | (m, 4H). | | | |
| 113 | 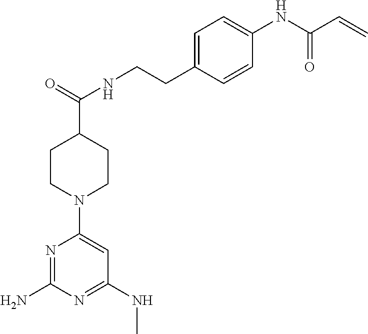 [SMILES: C=CC(=O)Nc1ccc(CCNC(=O)C2CCN(c3cc(NC)nc(N)n3)CC2)cc1] 1-(2-Amino-6-methylamino- pyrimidin-4-yl)-piperidine-4- carboxylic acid [2-(4- acryloylamino-phenyl)-ethyl]- amide | | B | B | 1.373 [424.2] |
| 114 | 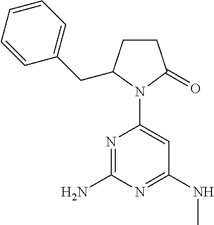 [SMILES: CNc1cc(N2C(=O)CCC2Cc2ccccc2)nc(N)n1] 1-(2-Amino-6-methylamino- pyrimidin-4-yl)-5-benzyl- pyrrolidin-2-one | 1H NMR (400 MHz, DMSO-d6) d 7.33-7.27 (m, 4H), 7.27-7.20 (m, 1H), 6.81- 6.72 (m, 2H), 5.95 (s, 2H), 4.80-4.73 (m, 1H), 3.08 (dd, J = 12.8, 2.9 Hz, 1H), 2.80-2.71 (m, 4H), 2.36- 2.16 (m, 2H), 1.99- 1.87 (m, 1H), 1.78-1.70 (m, 1H). | B | B | 1.144 [298.2] |
| 115 | 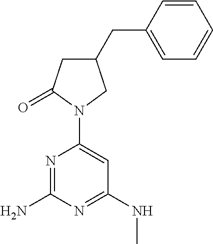 [SMILES: CNc1cc(N2CC(Cc3ccccc3)CC2=O)nc(N)n1] 1-(2-Amino-6-methylamino- pyrimidin-4-yl)-4-benzyl- pyrrolidin-2-one | 1H NMR (500 MHz, DMSO-d6) d 12.83-12.57 (m, 1H), 7.34-7.28 (m, 2H), 7.26- 7.19 (m, 3H), 6.86- 6.58 (m, 2H), 5.97-5.74 (m, 1H), 3.93-3.87 (m, 1H), 3.48 (dd, J = 11.0, 6.3 Hz, 1H), 2.82-2.75 (m, 1H), 2.74- 2.63 (m, 5H), 2.58 (dd, J = 16.6, 7.6 Hz, 1H), 2.34 (dd, J = 16.5, 7.1 Hz, 1H). | B | B | 1.158 [298.2] |
| 116 | 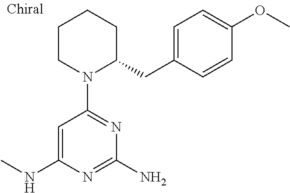 6-[(R)-2-(4-Methoxy-benzyl)- piperidin-1-yl]-N4-methyl- pyrimidine-2,4-diamine | | A | | 1.849 [328.2] |
| 117 | 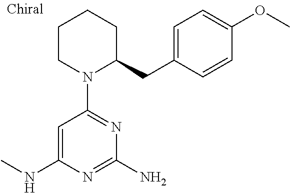 6-[(S)-2-(4-Methoxy-benzyl)- piperidin-1-yl]-N4-methyl- pyrimidine-2,4-diamine | | A | A | 1.848 [328.2] |
| 118 | 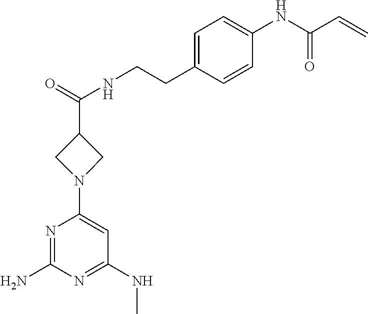 [SMILES: C=CC(=O)Nc1ccc(CCNC(=O)C2CN(c3cc(NC)nc(N)n3)C2)cc1] 1-(2-Amino-6-methylamino- pyrimidin-4-yl)-azetidine-3- carboxylic acid [2-(4- acryloylamino-phenyl)-ethyl]- amide | 1H NMR (400 MHz, DMSO-d6) δ 10.05 (s, 1H), 8.12 (t, J = 5.6 Hz, 1H), 7.66-7.53 (m, 2H), 7.34 (s, 2H), 7.20-7.09 (m, 2H), 6.42 (dd, J = 17.0, 10.1 Hz, 1H), 6.23 (dd, J = 17.0, 2.1 Hz, 1H), 5.73 (dd, J = 10.1, 2.1 Hz, 1H), 4.80 (s, 1H), 4.13 (t, J = 8.7 Hz, 2H), 3.99 (d, J = 8.4 Hz, 2H), 3.41 (s, 1H), 2.77 (d, J = 4.6 Hz, 3H), 2.69 (t, J = 7.3 Hz, 2H). | B | B | 1.287 [396.2] |
| 119 | 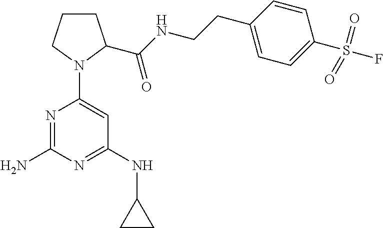 [SMILES: Nc1nc(NC2CC2)cc(N2CCCC2C(=O)NCCc2ccc(S(=O)(=O)F)cc2)n1] 4-(2-{[1-(2-Amino-6- cyclopropylamino-pyrimidin-4- yl)-pyrrolidine-2-carbonyl]- amino}-ethyl)-benzenesulfonyl fluoride | 1H NMR (400 MHz, DMSO-d6 90° C.) d 8.02-7.95 (m, 2H), 7.90- 7.73 (m, 2H), 7.63- 7.56 (m, 2H), 7.34-6.94 (m, 2H), 5.10 (s, 1H), 4.48-4.28 (m, 1H), 3.64-3.53 (m, 1H), 3.53- 3.42 (m, 2H), 3.42- 3.31 (m, 1H), 2.59-2.51 (m, 1H), 2.21-2.07 (m, 1H), 1.95- 1.84 (m, 3H), 1.60- 1.47 (m, 2H), 0.86-0.75 (m, 2H), 0.61-0.49 | B | | 1.248 [449.2] |
| | | (m, 2H). | | | |
| 120 | 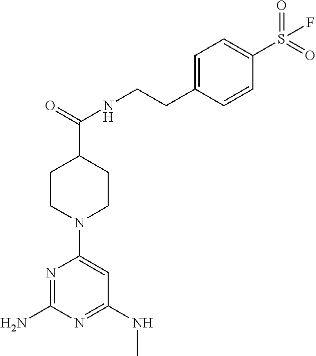 [SMILES: CNc1cc(N2CCC(C(=O)NCCc3ccc(S(=O)(=O)F)cc3)CC2)nc(N)n1] 4-(2-{[1-(2-Amino-6- methylamino-pyrimidin-4-yl)- piperidine-4-carbonyl]-amino}- ethyl)-benzenesulfonyl fluoride | 1H NMR (500 MHz, DMSO-d6) δ 11.04 (s, 1H), 8.04 J = 8.4 Hz, 2H), 7.91 (t, J = 5.7 Hz, 1H), 7.60 (d, J = 8.2 Hz, 2H), 7.25 (d, J = 45.9 Hz, 2H), 5.24 (s, 1H), 3.43-3.20 (m 9H), 2.97 (d, J = 13.4 Hz, 2H), 2.89 (t, J = 6.8 Hz, 2H), 2.79 (d, J = 4.8 Hz, 3H), 2.38 (tt, J = 11.3, 4.0 Hz, 1H), 1.68 (dd, J = 13.6, 3.7 Hz, 2H), 1.43 (d, J = 13.5 Hz, 2H). | A | | 1.568 [437.2] |
| 121 | 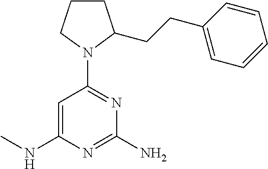 [SMILES: CNc1cc(N2CCCC2CCc2ccccc2)nc(N)n1] N4-methyl-6-[2-(2- phenylethyl)pyrrolidin-1- yl]pyrimidine-2,4-diamine | | | | |
| 122 | 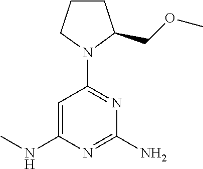 6-[(2S)-2- (methoxymethyl)pyrrolidin-1- yl]-N4-methyl-pyrimidine-2,4- diamine | | | | |
| 123 | 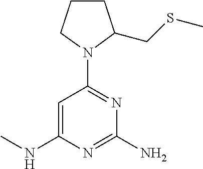 N4-methyl-6-[2- (methylsulfanylmethyl)pyrrolidin- 1-yl]pyrimidine-2,4-diamine | | | | |
| 124 | 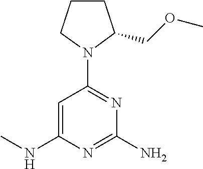 6-[(2R)-2- (methoxymethyl)pyrrolidin-1- yl]-N4-methyl-pyrimidine-2,4- diamine | | | | |
| 125 | 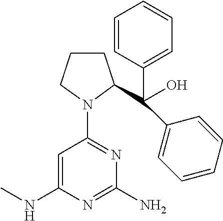 [SMILES: CNc1cc(N2CCC[C@H]2C(O)(c2ccccc2)c2ccccc2)nc(N)n1] [(2S)-1-[2-amino-6- (methylamino)pyrimidin-4- yl]pyrrolidin-2-yl]-diphenyl- methanol | | | | |
| 126 | 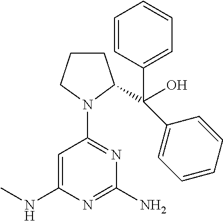 [SMILES: CNc1cc(N2CCC[C@@H]2C(O)(c2ccccc2)c2ccccc2)nc(N)n1] [(2R)-1-[2-amino-6- (methylamino)pyrimidin-4- yl]pyrrolidin-2-yl]-diphenyl- methanol | | | | |
| 127 | 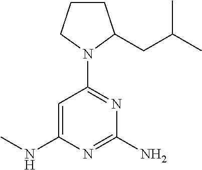 [SMILES: CNc1cc(N2CCCC2CC(C)C)nc(N)n1] 6-(2-isobutylpyrrolidin-1-yl)-N4- methyl-pyrimidine-2,4-diamine | | | | |
| 128 | 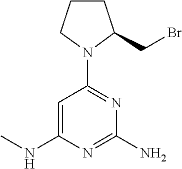 [SMILES: CNc1cc(N2CCC[C@H]2CBr)nc(N)n1] 6-[(2S)-2- (bromomethyl)pyrrolidin-1-yl]- N4-methyl-pyrimidine-2,4- diamine | | | | |
| 129 | 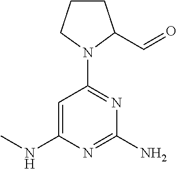 [SMILES: CNc1cc(N2CCCC2C=O)nc(N)n1] 1-[2-amino-6- (methylamino)pyrimidin-4- yl]pyrrolidine-2-carbaldehyde | | | | |
| 130 | 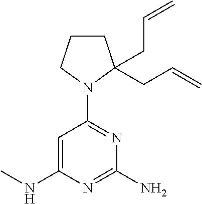 [SMILES: C=CCC1(CC=C)CCCN1c1cc(NC)nc(N)n1] 6-(2,2-diallylpyrrolidin-1-yl)-N4- methyl-pyrimidine-2,4-diamine | | | | |
| 131 | 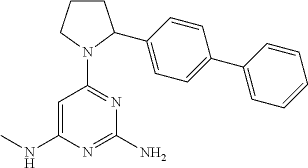 [SMILES: CNc1cc(N2CCCC2c2ccc(-c3ccccc3)cc2)nc(N)n1] N4-methyl-6-[2-(4- phenylphenyl)pyrrolidin-1- yl]pyrimidine-2,4-diamine | | | | |
| 132 | 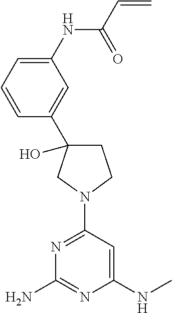 [SMILES: C=CC(=O)Nc1cccc(C2(O)CCN(c3cc(NC)nc(N)n3)C2)c1] N-[3-[1-[2-amino-6- (methylamino)pyrimidin-4-yl]-3- hydroxy-pyrrolidin-3- yl]phenyl]prop-2-enamide | | | | |
| 133 | 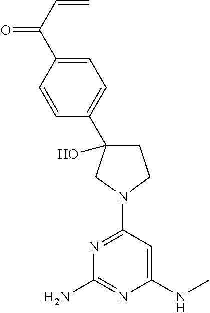 [SMILES: C=CC(=O)c1ccc(C2(O)CCN(c3cc(NC)nc(N)n3)C2)cc1] 1-[4-[1-[2-amino-6- (methylamino)pyrimidin-4-yl]-3- hydroxy-pyrrolidin-3- yl]phenyl]prop-2-en-1-one | | | | |
| 134 | 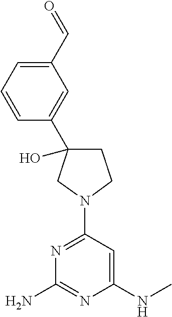 [SMILES: CNc1cc(N2CCC(O)(c3cccc(C=O)c3)C2)nc(N)n1] 3-[1-[2-amino-6- (methylamino)pyrimidin-4-yl]-3- hydroxy-pyrrolidin-3- yl]benzaldehyde | | | | |
| 135 | 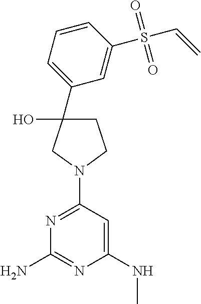 [SMILES: C=CS(=O)(=O)c1cccc(C2(O)CCN(c3cc(NC)nc(N)n3)C2)c1] 1-[2-amino-6- (methylamino)pyrimidin-4-yl]-3- (3- vinylsulfonylphenyl)pyrrolidin- 3-ol | | | | |
| 136 | 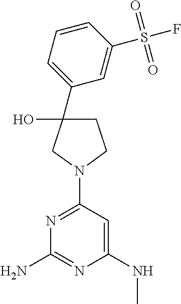 [SMILES: CNc1cc(N2CCC(O)(c3cccc(S(=O)(=O)F)c3)C2)nc(N)n1] 3-[1-[2-amino-6- (methylamino)pyrimidin-4-yl]-3- hydroxy-pyrrolidin-3- yl]benzenesulfonyl fluoride | | | | |
| 137 | 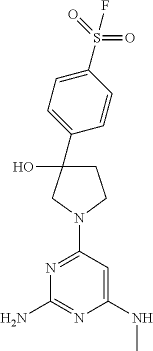 [SMILES: CNc1cc(N2CCC(O)(c3ccc(S(=O)(=O)F)cc3)C2)nc(N)n1] 4-[1-[2-amino-6- (methylamino)pyrimidin-4-yl]-3- hydroxy-pyrrolidin-3- yl]benzenesulfonyl fluoride | | | | |
| 138 | 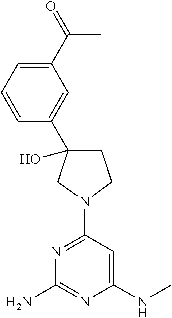 [SMILES: CNc1cc(N2CCC(O)(c3cccc(C(C)=O)c3)C2)nc(N)n1] 1-[3-[1-[2-amino-6- (methylamino)pyrimidin-4-yl]-3- hydroxy-pyrrolidin-3- yl]phenyl]ethanone | | | | |
| 139 | 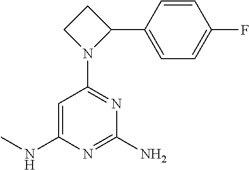 [SMILES: CNc1cc(N2CCC2c2ccc(F)cc2)nc(N)n1] 6-[2-(4-fluorophenyl)azetidin-1- yl]-N4-methyl-pyrimidine-2,4- diamine | | | | |
| 140 | 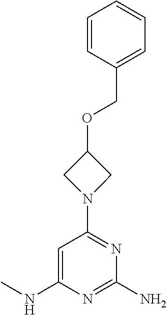 [SMILES: CNc1cc(N2CC(OCc3ccccc3)C2)nc(N)n1] 6-(3-benzyloxyazetidin-1-yl)- N4-methyl-pyrimidine-2,4- diamine | | | | |
| 141 | 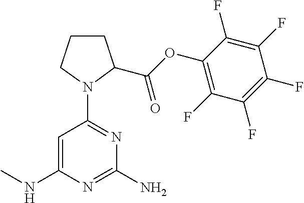 [SMILES: CNc1cc(N2CCCC2C(=O)Oc2c(F)c(F)c(F)c(F)c2F)nc(N)n1] (2,3,4,5,6-pentafluorophenyl) 1-[2-amino-6- (methylamino)pyrimidin-4- yl]pyrrolidine-2-carboxylate | | | | |
| 142 | 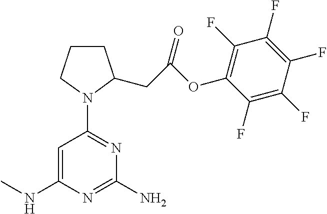 [SMILES: CNc1cc(N2CCCC2CC(=O)Oc2c(F)c(F)c(F)c(F)c2F)nc(N)n1] (2,3,4,5,6-pentafluorophenyl) 2-[1-[2-amino-6- (methylamino)pyrimidin-4- yl]pyrrolidin-2-yl]acetate | | | | |
| 143 | 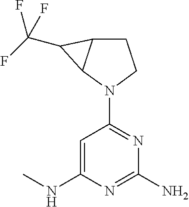 [SMILES: CNc1cc(N2CCC3C2C3C(F)(F)F)nc(N)n1] N4-methyl-6-[6- (trifluoromethyl)-2- azabicyclo[3.1.0]hexan-2- yl]pyrimidine-2,4-diamine | | | | |
| 144 | 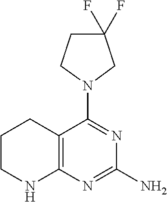 [SMILES: Nc1nc2c(c(N3CCC(F)(F)C3)n1)CCCN2] 4-(3,3-Difluoro-pyrrolidin-1-yl)- 5,6,7,8-tetrahydro-pyrido[2,3- d]pyrimidin-2-ylamine | 1H NMR (400 MHz, DMSO-d6) d 7.23 (s, 1H), 6.78 (s, 2H), 3.98 (t, J = 13.3 Hz, 2H), 3.79 (t, J = 7.3 Hz, 2H), 3.25-3.17 (m, 2H), 2.59 (t, J = 6.2 Hz, 2H), 2.47- 2.34 (m, 2H), 1.74- 1.66 (m, 2H). | A | B | 1.055 [256.3] |
| 145 | 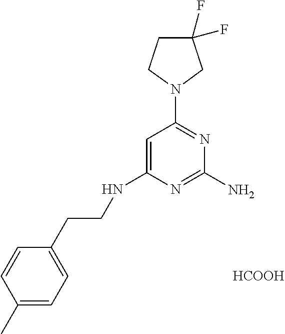 [SMILES: Cc1ccc(CCNc2cc(N3CCC(F)(F)C3)nc(N)n2)cc1 ; "HCOOH"] 6-(3,3-Difluoro-pyrrolidin-1-yl)- N4-(2-p-tolyl-ethyl)-pyrimidine- 2,4-diamine-formiate | ¹H NMR (400 MHz, DMSO-d6) δ 11.13 (s, 1H), 7.43 (s, 2H), 7.14 (q, J = 7.9 Hz, 4H), 5.07 (s, 1H), 3.88 (t, J = 12.9 Hz, 2H), 3.66 (s, 2H), 3.43 (d, J = 6.8 Hz, 2H), 2.81 (t, J = 7.2 Hz, 2H), 2.53 (m, 2H), 2.28 (s, 3H). | B | | 1.814 [334.2] |
| 146 | 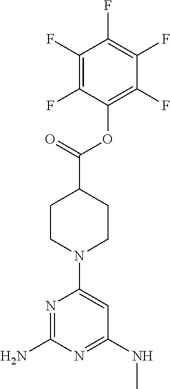 [SMILES: CNc1cc(N2CCC(C(=O)Oc3c(F)c(F)c(F)c(F)c3F)CC2)nc(N)n1] 1-(2-Amino-6-methylamino- pyrimidin-4-yl)-piperidine-4- carboxylic acid pentafluorophenyl ester | | | | |
| 147 | 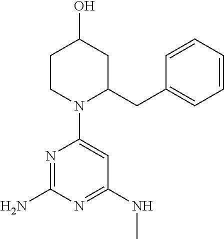 [SMILES: CNc1cc(N2CCC(O)CC2Cc2ccccc2)nc(N)n1] 1-(2-Amino-6-methylamino- pyrimidin-4-yl)-2-benzyl- piperidin-4-one | | | | |
| 148 | 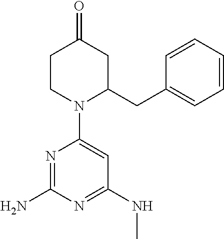 [SMILES: CNc1cc(N2CCC(=O)CC2Cc2ccccc2)nc(N)n1] | | | | |
| 149 | 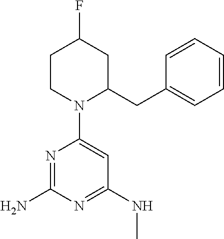 [SMILES: CNc1cc(N2CCC(F)CC2Cc2ccccc2)nc(N)n1] 6-(2-Benzyl-4 fluoro-piperidin- 1-yl)-N4-methyl-pyrimidine-2,4- diamine | | | | |
| 150 | 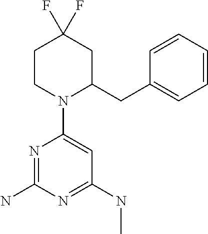 [SMILES: CNc1cc(N2CCC(F)(F)CC2Cc2ccccc2)nc(N)n1] 6-(2-Benzyl-4,4-difluoro- piperidin-1-yl)-N4-methyl- pyrimidine-2,4-diamine | | | | |

For the avoidance of doubt, should chemical name and chemical structure of the herein illustrated compounds or substituents not correspond by mistake, and the mistake not be obvious and should the identity of the compound not be deductible from the MS and/or NMR data, then the chemical structure shall be regarded as unambiguously defining the compound.

The compounds according to the present invention have, beyond their favourable MTH1 inhibitory activity, surprisingly favourable solubility and microsomal stability properties, as illustrated by the detain Table 2 below.

In particular, solubility and microsomal stability of the compounds are improved as compared to compound “TH287”, the compound having the best MTH1 inhibitory activity described in the earlier mentioned Nature publication by Gad, Helleday et al., Nature Vol. 508, 10 Apr. 2014, p. 215 onwards.

TABLE 2
Exemplary compounds in comparison with reference compound

| | | | Microsomal | |
|---|---|---|---|---|
| | | | stability | |
| | | | [μl/min/ | |
| | | | mg prot.] | |
| | IC₅₀ | Solubility | human | |
| Example | [μM] | [μg/ml] | mouse | |
| Reference example | <0.001 | 22 | 32 | 444 |
| 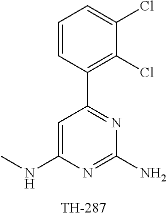 [SMILES: CNc1cc(-c2cccc(Cl)c2Cl)nc(N)n1] | | | | |
| 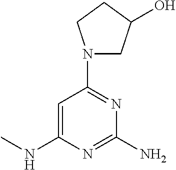 1-(2-Amino-6-methylamino- pyrimidin-4-yl)-pyrrolidin-3-ol- formate | 0.0013 | >1000 | <10 | <10 |
| 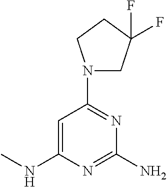 [SMILES: CNc1cc(N2CCC(F)(F)C2)nc(N)n1] 6-(3,3-Difluoro-pyrrolidin-1-yl)- N4-methyl-pyrimidine-2,4- diamine | <0.001 | >1000 | <10 | 43 |
| 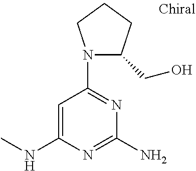 [SMILES: CNc1cc(N2CCC[C@@H]2CO)nc(N)n1] [(R)-1-(2-Amino-6-methylamino- pyrimidin-4-yl)-pyrrolidin-2-yl]- methanol formate | 0.002 | >1000 | <10 | <10 |
| 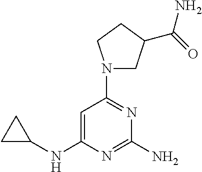 [SMILES: NC(=O)C1CCN(c2cc(NC3CC3)nc(N)n2)C1] 1-(2-Amino-6-cyclopropylamino- pyrimidin-4-yl)-pyrrolidine-3- carboxylic acid amide-formate | 0.007 | >1000 | <10 | 13 |

As demonstrated above, compounds according to the present invention do not only have excellent inhibitory properties (IC50 values), good solubility and microsomal stability. What's more, various examples showed very favourable (long) residence times on the target—at least by a factor 10 longer than the “TH287” and “TH588” compound disclosed as having the best MTH1 inhibitory activity in the earlier mentioned Nature publication by Gad, Helleday et al., Nature Vol. 508, 10 Apr. 2014, p. 215 onwards, as illustrated in Table 3 below. The residence time is calculated on the basis of the KD values indicated in Table 1.

TABLE 3
Overview of residence times in comparison to Reference example

| Example | Residence time |
|---|---|
| Reference example | <1 min (54 seconds) |
| TH-287 (Helleday, see above) | |
| Reference example | <1 min (18 seconds) |
| TH-588 (Helleday, see above) | |
| Compound 95 | ≥10 min |
| Compound 116 | ≥10 min |
| Compound 100 | ≥10 min |
| Compound 92 | ≥10 min |
| Compound 105 | ≥10 min |
| Compound 98 | ≥10 min |
| Compound 108 | ≥10 min |
| Compound 120 | ≥10 min |

1. A compound of Formula I or Formula II 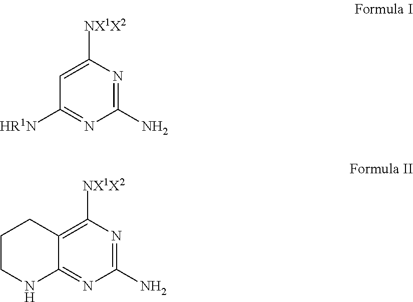
or a pharmaceutically acceptable salt, stereoisomer, tautomer or solvate thereof,
for use in the treatment of cancer wherein
R¹ represents ALK1 optionally substituted by one or more substituents E¹, ALK2 optionally substituted by one or more substituents E³, or ALK3 optionally substituted by one or more substituents E⁴;
E¹, E³, E⁴ each being independently selected from halogen, hydroxy, oxo (═O), nitro, —CN, —C(O)R^{E1}, —C(O)OR^{E2}, —C(O)NR^{E3}R^{E4}, —OR^{E5}, —OC(O)R^{E6}, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21}, and aryl optionally substituted by one or more substituents E¹¹;
E¹¹ being independently selected from ALK1 optionally substituted by one or more substituents E²¹, halogen, hydroxy, oxo (═O), nitro, —CN, —C(O)R^{E1}, —C(O)OR^{E2}, —C(O)NR^{E3}R^{E4}, —OR^{E5}, —OC(O)R, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21};
E²¹ being independently selected from halogen, hydroxy, oxo (═O), nitro, —CN, —C(O)R^{E1}, —C(O)OR^{E2}, —C(O)NR^{E3}R^{E4}, —OR^{E5}, —OC(O)R^{E6}, —NR^{E7}C(O)R^{E8}, —NR^{E9}C(O)OR^{E10}, —NR^{E11}C(O)NR^{E12}R^{E13}, —NR^{E16}S(O)₂R^{E17}, —OS(O)₂R^{E18}, —S(O)ₓR^{E19}, and —S(O)₂NR^{E20}R^{E21};
R^{E1}, R^{E2}, R^{E3}, R^{E4}, R^{E5}, R^{E6}, R^{E7}, R^{E8}, R^{E9}, R^{E10}, R^{E11}, R^{E12}, R^{E13}, R^{E16}, R^{E17}, R^{E18}, R^{E19}, R^{E20} and R^{E21} each being independently selected from H, ALK1, ALK2, ALK3, and aryl, each of which may be optionally substituted by one or more of halogen, hydroxy, oxo (═O), nitro, —CN, and C₁-C₁₂ alkoxy;
wherein R^{E19} may also be selected from F,
X¹ and X² together with the N to which they are attached form a heterocycle which is selected from: 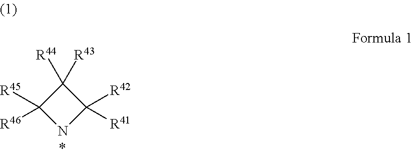
wherein R⁴¹, R⁴², R⁴³, R⁴, R⁴⁵, and R⁴⁶ are independently selected from H, hydroxy, nitro, —CN, halogen, ALK1 optionally substituted by one or more substituents M⁴¹, aryl optionally substituted by one or more substituents M⁴², heterocyclyl optionally substituted by one or more substituents M⁴³, ALK2 optionally substituted by one or more substituents M⁴⁴, ALK3 optionally substituted by one or more substituents M⁴⁵, —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹ and —S(O)₂NR⁴²⁰R⁴²¹,
or R⁴¹ with R⁴², R⁴³ with R⁴ or R⁴⁵ with R⁴⁶ together form ═O or ═S,
or a combination of R⁴³ and R⁴⁴, R⁴¹ and R⁴², or R⁴⁵ and R⁴⁶ together with the C atom to which they are attached form a 4- to 10-membered carbocyclic or heterocyclic ring system, which ring system is optionally substituted by one or more substituents M⁴⁶,
or a combination of R⁴¹ with R⁴³ or R⁴³ with R⁴⁵ together with the C atoms to which they are attached form a 3- or 4- to 10-membered carbocyclic or heterocyclic ring system, which ring system is optionally substituted by one or more substituents M⁴⁷, R⁴⁰¹, R⁴⁰², R⁴⁰³, R⁴⁰⁴, R⁴⁰⁵, R⁴⁰⁶, R⁴⁰⁷, R⁴⁰⁸, R⁴⁰⁹, R⁴¹⁰, R⁴¹¹, R⁴¹², R⁴¹³, R⁴¹⁶, R⁴¹⁷, R⁴¹⁸, R⁴¹⁹, R⁴²⁰, R⁴²¹, each being independently selected from H, ALK1 optionally substituted by one or more substituents M⁴⁸, aryl optionally substituted by one or more substituents M⁴⁹,
wherein R⁴¹⁹ in —S(O)₂R⁴¹⁹ may also be F or vinyl,
wherein R⁴⁰¹, R⁴⁰⁵, R⁴⁰⁸ may each independently also be vinyl,
M⁴¹, M⁴⁴, M⁴⁵ and M⁴⁸ each being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹8, —S(O)ₓR⁴¹⁹, —S(O)₂NR⁴²⁰R⁴²¹ and aryl optionally substituted by one or more substituents M^{49a},
M⁴² being independently selected from, halogen, nitro, hydroxy, —C(O)R⁴⁰¹, —C(O)OR⁴⁰², —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹, ALK1 optionally substituted by one or more substituents M^{48a} and aryl optionally substituted by one or more substituents M^{49a};
M⁴³, M⁴⁹ each being independently selected from, halogen, nitro, hydroxy, —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR^{412R413}, —NR⁴¹⁶S(O)₂R⁴¹⁷, —OS(O)₂R⁴¹⁸, —S(O)ₓR⁴¹⁹, —S(O)₂NR⁴²⁰R⁴²¹ and ALK1 optionally substituted by one or more substituents M^{48a};
M⁴⁶ and M⁴⁷ each being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹, ALK1 optionally substituted by one or more substituents M^{48a} and aryl optionally substituted by one or more substituents M^{49a};
M⁴⁸a being independently selected from halogen, —CN, nitro, hydroxy, oxo (═O), —C(O)R⁴⁰¹, —C(O)OR⁴⁰², —C(O)NR⁴⁰³R⁴⁰⁴, —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, and —S(O)₂NR⁴²⁰R⁴²¹;
M^{49a} being independently selected from halogen, nitro, hydroxy, oxo (═O), —C(O)R⁴⁰¹, —C(O)OR⁴⁰², —OR⁴⁰⁵, —OC(O)R⁴⁰⁶, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷, —OS(O)₂R⁴¹⁸, —S(O)ₓR⁴¹⁹, —S(O)₂NR⁴²⁰R⁴²¹ and ALK1, which is optionally substituted by one or more of halogen, —CN, nitro, hydroxy or C₁₋₁₂ alkoxy;
with the proviso that any N-atom, if present, in addition to the N-atom depicted in above Formula 1 is comprised in the form of a substituent selected from nitro, —CN, —C(O)NR⁴⁰³R⁴⁰⁴, —NR⁴⁰⁷C(O)R⁴⁰⁸, —NR⁴⁰⁹C(O)OR⁴¹⁰, —NR⁴¹¹C(O)NR⁴¹²R⁴¹³, —NR⁴¹⁶S(O)₂R⁴¹⁷ and —S(O)₂NR⁴²⁰R⁴²¹; 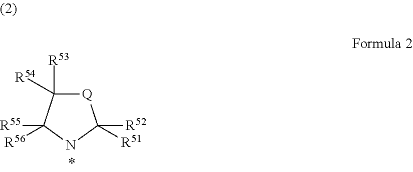
wherein Q is selected from O, S, and CR⁵⁷R⁵⁸,
wherein R⁵¹, R⁵², R⁵³, R⁵⁴, R⁵⁵, R⁵⁶, R⁵⁷, and R⁵⁸ are independently selected from H, hydroxy, nitro, —CN, halogen, ALK1 optionally substituted by one or more substituents M⁵¹, aryl optionally substituted by one or more substituents M⁵², heterocyclyl optionally substituted by one or more substituents M⁵³, ALK2 optionally substituted by one or more substituents M⁵⁴, ALK3 optionally substituted by one or more substituents M⁵⁵, —C(O)R⁵⁰¹, —C(O)OR⁵², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵⁰¹, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹,
or R⁵¹ with R⁵², R⁵³ with R⁵⁴, R⁵⁵ with R⁵⁶ or R⁵⁷ with R⁵⁸ together form ═O or ═S, or a combination of R⁵¹ and R⁵², R⁵³ and R⁵⁴, R⁵⁵ and R⁵⁶ or R⁵⁷ and R⁵⁸ together with the C atom to which they are attached form a 4- to 10-membered carbocyclic or heterocyclic ring system, which ring system is optionally substituted by one or more substituents M⁵⁶,
or a combination of R⁵¹ with R⁵⁷, R⁵³ with R⁵⁷, or R⁵³ with R⁵⁵ together with the C atoms to which they are attached form a 3-, 4-, 5-, 6-, 7-, 8-, 9-, or 10-membered carbocyclic or heterocyclic ring system, which ring system is optionally substituted by one or more substituents M⁵⁷,
R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁶, R⁵¹⁷, R⁵¹⁸, R⁵¹⁹, R⁵²⁰, and R⁵²¹ each being independently selected from H, ALK1 optionally substituted by one or more substituents M^{58a} and aryl optionally substituted by one or more substituents M⁵⁹;
wherein R⁵¹⁹ in —S(O)₂R⁴¹⁹ may also be F or vinyl,
wherein R⁵⁰¹, R⁵⁰⁵ and R⁵⁰⁸ may each independently also be vinyl,
M⁵¹, M⁵⁴, M⁵⁵ and M^{58a} each being independently selected from halogen, —CN, nitro, hydroxy, oxo (═O), —C(O)R⁵⁰¹, —C(O)OR⁵⁰², —C(O)NR⁵⁰³R⁵, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵C(O)NR⁵¹²R⁵¹³, —NR⁵¹⁶S(O)₂R⁵¹⁷, —OS(O)₂R⁵¹, —S(O)ₓR⁵¹⁹, —S(O)₂NR⁵²R⁵²¹ and aryl optionally substituted by one or more substituents M^{59a};
M⁵² being independently selected from halogen, nitro, hydroxy, —C(O)R⁵⁰¹, —C(O)OR⁵², —OR⁵⁰⁵, —OC(O)R⁵⁰⁶, —NR⁵⁰⁷C(O)R⁵⁰⁸, —NR⁵⁰⁹C(O)OR⁵¹, —NR⁵¹¹C(O)NR⁵¹²R⁵¹³, —NR⁵¹⁶S(O)₂R⁵¹⁷, —OS(O)₂R⁵¹⁸, —S(O)R⁵¹⁹, —S(O)₂NR⁵²⁰R⁵²¹, ALK1 optionally substituted by one or more substituents M^{58b}, and aryl optionally substituted by one or more substituents M^{59a};
M⁵³ and M⁵⁹ each being independently selected from halogen, nitro, hydroxy, —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵²¹, and ALK1 optionally substituted by one or more substituents M^{58b};
M⁵⁶ and M⁵⁷ each being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, —S(O)₂NR⁵²⁰R⁵², ALK1 optionally substituted by one or more substituents M^{58b} and aryl optionally substituted by one or more substituents M^{59a};
M^{58b} being independently selected from halogen, —CN, nitro, hydroxy, oxo (═O), —C(O)R⁵⁰¹, —C(O)OR⁵⁰², —C(O)NR⁵⁰³R⁵⁰⁴, —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)ₓR⁵¹⁹, and —S(O)₂NR⁵²⁰R⁵²¹;
M^{59a} being independently selected from halogen, nitro, hydroxy, oxo (═O), —C(O)R⁵⁰¹, —C(O)OR⁵⁰², —OR⁵⁰⁵, —OC(O)R⁵⁰⁶, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, —OS(O)₂R⁵¹⁸, —S(O)R⁵¹⁹, —S(O)₂NR⁵²⁰R⁵²¹ and ALK1, which is optionally substituted by one or more of halogen, —CN, nitro, hydroxy or C₁₋₁₂ alkoxy;
with the proviso that any N-atom, if present, in addition to the N-atom depicted in above formula 2 is comprised in the form of a substituent selected from nitro, —CN, —C(O)NR⁵⁰³R⁵⁰⁴, —NR⁵⁰⁷C(O)R⁵⁰⁸, —NR⁵⁰⁹C(O)OR⁵¹⁰, —NR⁵¹¹C(O)NR⁵¹²R⁵¹³, —NR⁵¹⁶S(O)₂R⁵¹⁷, and —S(O)₂NR⁵²⁰R⁵²¹; 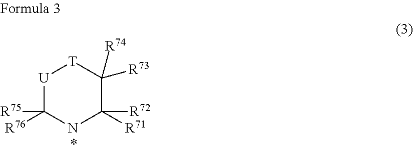
wherein
U is selected from CR⁷⁷R⁷⁸, O and S;
T is selected from CR⁸⁰R⁸¹, O, and S, with the proviso that only one of U and T may be selected from O and S; and
R⁷¹, R⁷², R⁷³, R⁷⁴, R⁷⁵, R⁷⁶, R⁷⁷, R⁷⁸, R⁸⁰ and R⁸¹ are independently selected from H, hydroxy, nitro, —CN, halogen, ALK1 optionally substituted by one or more substituents M⁷¹, aryl optionally substituted by one or more substituents M⁷², heterocyclyl optionally substituted by one or more substituents M⁷³, ALK2 optionally substituted by one or more substituents M⁷⁴, ALK3 optionally substituted by one or more substituents M⁷⁵, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, and —S(O)₂NR⁷²⁰R⁷²¹;
or a combination of R⁷¹ and R⁷², R⁷³ and R⁷⁴, R⁷⁵ and R⁷⁶, R⁷⁷ and R⁷⁸, or R⁸⁰ and R⁸¹ together form ═O or ═S,
or a combination of R⁷¹ and R⁷², R⁷³ and R⁷⁴, R⁷⁵ and R⁷⁶, R⁷⁷ and R⁷⁸, or R⁸⁰ and R⁸¹ together with the C atom to which they are attached form a 4- to 10-membered carbocyclic or heterocyclic ring system, which ring system is optionally substituted by one or more substituents M⁷⁶,
or a combination of R⁷² and R⁷⁴, R⁷⁴ and R⁸⁰, R⁸⁰ and R⁷⁸, or R⁷⁸ and R⁷⁶ together with the C atoms to which they are attached form a 3- or 4- to 10-membered carbocyclic or heterocyclic ring system, which ring system is optionally substituted by one or more substituents M⁷⁷,
R⁷⁰¹, R⁷⁰², R⁷⁰³, R⁷⁰⁴, R⁷⁰⁵, R⁷⁰⁶, R⁷⁰⁷, R⁷⁰⁸, R⁷⁰⁹, R⁷¹⁰, R⁷¹¹, R⁷¹², R⁷¹³, R⁷¹⁶, R⁷¹⁷, R⁷¹⁸, R⁷¹⁹, R⁷²⁰ and R⁷²¹ are independently selected from H, ALK1 optionally substituted by one or more substituents M^{78a} and aryl optionally substituted by one or more substituents M⁷⁹;
wherein R⁷¹⁹ in —S(O)₂R⁷¹⁹ may also be F or vinyl,
wherein R⁷⁰¹, R⁷⁰⁵ and R⁷⁰⁸ may each independently also be vinyl,
M⁷¹, M⁷⁴, M⁷⁵ and M^{78a} are each independently selected from hydroxy, oxo (═O), nitro, —CN, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and aryl optionally substituted by one or more substituents M^{79a};
M⁷² each independently selected from hydroxy, nitro, halogen, —C(O)R⁷⁰¹, —C(O)OR⁷⁰², —OR⁷⁰⁵, —OC(O)R⁷⁰⁶, —NR⁷⁰⁷C(O)R⁷⁰, —NR⁷⁰⁹C(O)OR⁷¹⁰, —NR⁷¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹, ALK1 optionally substituted by one or more substituents M^{78b} and aryl optionally substituted by one or more substituents M^{79a};
M⁷³ and M⁷⁹ each independently selected from hydroxy, nitro, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and ALK1 optionally substituted by one or more substituents M^{78b};
M⁷⁶ and M⁷⁷ each independently selected from hydroxy, oxo (═O), nitro, —CN, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR¹²R⁷¹, —NR⁷¹⁶S(O)₂R⁷¹⁷, —OS(O)₂R⁷¹, —S(O)ₓR⁷¹⁹, —S(O)₂NR⁷²OR⁷²¹, ALK1 optionally substituted by one or more substituents M^{78b} and aryl optionally substituted by one or more substituents M^{79a};
M^{78b} each independently selected from hydroxy, oxo (═O), nitro, —CN, halogen, —C(O)R⁷⁰¹, —C(O)OR⁷⁰², —C(O)NR⁷⁰³R⁷⁰⁴, —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, and —S(O)₂NR⁷²⁰R⁷²¹;
M^{79a} each independently selected from hydroxy, oxo (═O), nitro, halogen, —C(O)R⁷⁰¹, —C(O)OR⁷⁰², —OR⁷⁰⁵, —OC(O)R⁷⁰⁶, —NR⁷⁰⁷C(O)R⁷⁰⁸, —NR⁷⁰⁹C(O)OR⁷¹⁰, —NR⁷¹¹C(O)NR⁷¹²R⁷¹³, —NR⁷¹⁶S(O)₂R⁷¹⁷, —OS(O)₂R⁷¹⁸, —S(O)ₓR⁷¹⁹, —S(O)₂NR⁷²⁰R⁷²¹ and ALK1, which is optionally substituted by one or more of halogen, —CN, nitro, hydroxy or C₁₋₁₂ alkoxy;
with the proviso that any N-atom, if present, in addition to the N-atom depicted in above Formula 3 is comprised in the form of a substituent selected from nitro, —CN, —C(O)NR⁷⁰³R⁷⁰⁴, —NR⁷⁰⁷C(O)R⁷⁰⁸, —NR⁷⁰⁹C(O)OR⁷¹⁰, —NR⁷¹¹C(O)NR R⁷¹²R⁷¹³, —NR⁷¹⁶S(O)₂R⁷¹⁷ and —S(O)₂NR⁷²⁰R⁷²¹
and 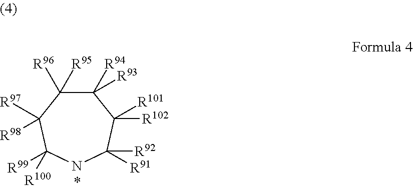
wherein
R⁹¹, R⁹², R⁹³, R⁹⁴, R⁹⁵, R⁹⁶, R⁹⁷, R⁹⁸, R⁹⁹, R¹⁰⁰, R¹⁰¹ and R¹⁰² are independently selected from H, hydroxy, nitro, —CN, halogen, ALK1 optionally substituted by one or more substituents M⁹¹, aryl optionally substituted by one or more substituents M⁹², heterocyclyl optionally substituted by one or more substituents M⁹³, ALK2 optionally substituted by one or more substituents M⁹⁴, ALK3 optionally substituted by one or more substituents M⁹⁵, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹;
or a combination of R⁹¹ and R⁹², R⁹³ and R⁹⁴, R⁹⁵ and R⁹⁶, R⁹⁷ and R⁹⁸, R⁹⁹ and R¹⁰⁰, or R¹⁰¹ and R¹⁰² together forms ═O or ═S,
or R¹⁰¹ and R⁹⁷ together form an oxygen bridge member (—O—),
or a combination of R⁹¹ and R⁹², R⁹³ and R⁹⁴, R⁹⁵ and R⁹⁶, R⁹⁷ and R⁹⁸, or R⁹⁹ and R¹⁰⁰ together with the C atom to which they are attached form a 4- to 10-membered carbocyclic or heterocyclic ring system, which ring system is optionally substituted by one or more substituents M⁹⁶,
or a combination of R⁹¹ and R¹⁰¹, R⁹³ and R¹⁰¹, R⁹³ and R⁹⁵, R⁹⁵ and R⁹⁷, R⁹⁷ and R⁹⁹ together with the C atoms to which they are attached form a 3- or 4- to 10-membered carbocyclic or heterocyclic ring system, which ring system is optionally substituted by one or more substituents M⁹⁷,
R⁹⁰¹, R⁹⁰², R⁹⁰³, R⁹⁰⁴, R⁹⁰⁵, R⁹⁰⁶, R⁹⁰⁷, R⁹⁰⁸, R⁹⁰⁹, R⁹¹⁰, R⁹¹¹, R⁹¹², R⁹¹³, R⁹¹⁶, R⁹¹⁷, R⁹¹⁸, R⁹¹⁹, R⁹²⁰ and R⁹²¹ are each independently selected from H, ALK1 optionally substituted by one or more substituents M^{98a} and aryl optionally substituted by one or more substituents M⁹⁹;
wherein R⁹¹⁹ in —S(O)₂R⁹¹⁹ may also be F or vinyl,
wherein R⁹⁰¹, R⁹⁰⁵ and R⁹⁰⁸ may each independently also be vinyl,
M⁹¹, M⁹⁴, M⁹⁵ and M^{98a} are each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and aryl optionally substituted by one or more substituents M^{99a};
M⁹² is each independently selected from hydroxy, nitro, halogen, —C(O)R⁹⁰¹, —C(O)OR⁹⁰², —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1 optionally substituted by one or more substituents M^{98b};
M⁹³ and M⁹⁹ are each independently selected from hydroxy, nitro, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1 optionally substituted by one or more substituents M^{98b};
M⁹⁶ and M⁹⁷ are each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹, ALK1 optionally substituted by one or more substituents M^{98b} and aryl optionally substituted by one or more substituents M^{99a};
M^{98b} each independently selected from hydroxy, oxo (═O), nitro, —CN, halogen, —C(O)R⁹⁰¹, —C(O)OR⁹⁰², —C(O)NR⁹⁰³R⁹⁰⁴, —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, and —S(O)₂NR⁹²⁰R⁹²¹,
M^{99a} each independently selected from hydroxy, oxo (═O), nitro, halogen, —C(O)R⁹⁰¹, —C(O)OR⁹⁰², —OR⁹⁰⁵, —OC(O)R⁹⁰⁶, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷, —OS(O)₂R⁹¹⁸, —S(O)ₓR⁹¹⁹, —S(O)₂NR⁹²⁰R⁹²¹ and ALK1, which is optionally substituted by one or more of halogen, —CN, nitro, hydroxy or C₁₋₁₂ alkoxy,
with the proviso that any N-atom, if present, in addition to the N-atom depicted in above Formula 4 is comprised in the form of a substituent selected from nitro, —CN, —C(O)NR⁹³R⁹⁰⁴, —OR⁹⁰⁵, —OC(O)R⁹⁰, —NR⁹⁰⁷C(O)R⁹⁰⁸, —NR⁹⁰⁹C(O)OR⁹¹⁰, —NR⁹¹¹C(O)NR⁹¹²R⁹¹³, —NR⁹¹⁶S(O)₂R⁹¹⁷ and —S(O)₂NR⁹²⁰R⁹²¹;
and wherein
ALK1 denotes branched or unbranched alkyl having 1, 2, 3, 4, 5, 6, 7, 8, 9, 10, 11 or 12 carbon atoms, cycloalkyl having 3, 4, 5, 6, 7, 8, 9, 10, 11 or 12 carbon atoms, or cycloalkyl substituted alkyl groups having 4, 5, 6, 7, 8, 9, 10, 11 or 12 carbon atoms in total,
ALK2 denotes olefinic groups having 2, 3, 4, 5, 6, 7, 8, 9, 10, 11 or 12 carbon atoms and having one or more double bonds, and includes acyclic branched and unbranched C₂-C₁₂ carbon chains with one or more double bonds, carbocycles having 5, 6, 7, 8, 9 or 10 carbon atoms and one or more double bonds with or without side chains, cycloalkyl substituted acyclic branched and unbranched carbon chains having 5, 6, 7, 8, 9, 10, 11 or 12 carbon atoms in total and cycloalkenyl substituted alkyl moieties having 6, 7, 8, 9, 10, 11 or 12 carbon atoms in total,
ALK3 denotes branched or unbranched alkynyl having 2, 3, 4, 5, 6, 7, 8, 9, 10, 11 or 12 carbon atoms or cycloalkyl substituted alkynyl having 5, 6, 7, 8, 9, 10, 11 or 12 carbon atoms in total, and
x is 0, 1 or 2.

2. Compound according to Formula I according to claim 1, wherein —NHR¹ is methylamino.

3. Compound according to Formula I according to claim 1, wherein —NHR¹ is cyclopropylamino.

4. Compound according to Formula I or II according to claim 1, wherein X¹ and X² together with the N to which they are attached form a heterocycle according to Formula 2, wherein Q is CR⁵⁷R⁵⁸.

5. Compound according to Formula I or II according to claim 1, wherein X¹ and X² together with the N to which they are attached form a heterocycle according to Formula 3, wherein U is CR⁷⁷R⁷⁸ and T is CR⁸⁰R⁸¹.

6. Compound according to Formula I or II according to claim 1, wherein X¹ and X² together with the N to which they are attached form a heterocycle according to Formula 1, wherein at least one of R⁴¹, R⁴², R⁴³, R⁴, R⁴⁵, and R⁴⁶ is selected from —O—CH₃, —O—CH₂—CH₃, —O—(C₁₋₆ alkyl), —O-ALK1, —CH₂—O—CH₃, —(CH₂)₂₋₄—O—(CH₂)₀₋₄CH₃, —CH₂—S—CH₃, —OH, —CH₂—OH, —(CH₂)₂₋₄—OH, —CF₃, —CH₂—Br, —(CH₂)₂₋₄—Br, —F, —Cl, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—S(O)₂F, —C(O)—NH—(CH₂)₂—(C₆H₄)—NH—C(O)—O—C(CH₃)₃, —C(O)—NH—(CH₂)₂—(C₆H₄)—NH—C(O)—CH═CH₂, —(C₆H₄)—NH—C(O)—CH═CH₂, —(C₆H₄)—C(O)—CH═CH₂, —(C₆H₄)—CH═O, —(C₆H₄)—S(O)₂—CH═CH₂, —(C₆H₄)—F, —(C₆H₄)—S(O)₂F, —O—(CH₂)₂—(C₆H₅); —C(O)—O—(C₆F₅), —CH₂—C(O)—O—(C₆F₅), —CH═O, and allyl.

7. Compound according to Formula I or II according to claim 1, wherein X¹ and X² together with the N to which they are attached form a heterocycle according to Formula 2, wherein at least one of R⁵¹, R⁵², R⁵³, R⁵⁴, R⁵⁵, R⁵⁶, R⁵⁷, and R⁵⁸ is selected from —O—CH₃, —O—CH₂—CH₃, —O—(C₁₋₆ alkyl), —O-ALK1, —CH₂—O—CH₃, —(CH₂)₂₋₄—O—(CH₂)₀₋₄CH₃, —CH₂—S—CH₃, —OH, —CH₂—OH, —(CH₂)₂₋₄—OH, —CF₃, —CH₂—Br, —(CH₂)₂₋₄—Br, —F, —Cl, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—S(O)₂F, —C(O)—NH—(CH₂)₂—(C₆H₄)—NH—C(O)—O—C(CH₃)₃, —C(O)—NH—(CH₂)₂—(C₆H₄)—NH—C(O)—CH═CH₂, —(C₆H₄)—NH—C(O)—CH═CH₂, —(C₆H₄)—C(O)—CH═CH₂, —(C₆H₄)—CH═O, —(C₆H₄)—S(O)₂—CH═CH₂, —(C₆H₄)—F, —(C₆H₄)—S(O)₂F, —O—(CH₂)₂—(C₆H₅); —C(O)—O—(C₆F₅), —CH₂—C(O)—O—(C₆F₅), —CH═O, and allyl.

8. Compound according to Formula I or II according to claim 1, wherein X¹ and X² together with the N to which they are attached form a heterocycle according to Formula 3, wherein at least one of R⁷¹, R⁷², R⁷³, R⁷⁴, R⁷⁵, R⁷⁶, R⁷⁷, R⁷⁸, R⁸⁰ and R⁸¹ is selected from —O—CH₃, —O—CH₂—CH₃, —O—(C₁₋₆ alkyl), —O-ALK1, —CH₂—O—CH₃, —(CH₂)₂₋₄—O—(CH₂)₀₋₄CH₃, —CH₂—S—CH₃, —OH, —CH₂—OH, —(CH₂)₂₋₄—OH, —CF₃, —CH₂—Br, —(CH₂)₂₋₄—Br, —F, —Cl, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—S(O)₂F, —C(O)—NH—(CH₂)₂—(C₆H₄)—NH—C(O)—O—C(CH₃)₃, —C(O)—NH—(CH₂)₂—(C₆H₄)—NH—C(O)—CH═CH₂, —(C₆H₄)—NH—C(O)—CH═CH₂, —(C₆H₄)—C(O)—CH═CH₂, —(C₆H₄)—CH═O, —(C₆H₄)—S(O)₂—CH═CH₂, —(C₆H₄)—F, —(C₆H₄)—S(O)₂F, —O—(CH₂)₂—(C₆H₅); —C(O)—O—(C₆F₅), —CH₂—C(O)—O—(C₆F₅), —CH═O, and allyl.

9. Compound according to Formula I or II according to claim 1, wherein X¹ and X² together with the N to which they are attached form a heterocycle according to Formula 4, wherein at least one of R⁹¹, R⁹², R⁹³, R⁹⁴, R⁹⁵, R⁹⁶, R⁹⁷, R⁹⁸, R⁹⁹, R¹⁰⁰, R¹⁰¹ and R¹⁰² is selected from —O—CH₃, —O—CH₂—CH₃, —O—(C₁₋₆ alkyl), —O-ALK1, —CH₂—O—CH₃, —(CH₂)₂₋₄—O—(CH₂)₀₋₄CH₃, —CH₂—S—CH₃, —OH, —CH₂—OH, —(CH₂)₂₋₄—OH, —CF₃, —CH₂—Br, —(CH₂)₂₋₄—Br, —F, —Cl, substituted or unsubstituted phenyl, substituted or unsubstituted benzyl, chloro-benzyl, 2-chlorobenzyl, 3-chlorobenzyl, 4-chlorobenzyl, methoxy-benzyl, 2-methoxy-benzyl, 4-methoxy-benzyl, methyl-benzyl, 2-methyl-benzyl, 3-methyl-benzyl, 1-methyl-1-phenyl-ethyl, phenethyl, diphenyl-hydroxy-methyl (—C(OH)(C₆H₅)₂), benzofuranyl, 2-benzofuranyl, thiophenyl, thiophen-3-yl, substituted or unsubstituted methyl, substituted or unsubstituted ethyl, substituted or unsubstituted isopropyl, substituted or unsubstituted isobutyl, substituted or unsubstituted cyclopentyl, —CH₂—C(O)—O—C₄H₉, —C(O)—NH₂, —C(O)—NH—(C₆H₅), —C(O)—NH—(CH₂)₂—(C₆H₄)—S(O)₂F, —C(O)—NH—(CH₂)₂—(C₆H₄)—NH—C(O)—O—C(CH₃)₃, —C(O)—NH—(CH₂)₂—(C₆H₄)—NH—C(O)—CH═CH₂, —(C₆H₄)—NH—C(O)—CH═CH₂, —(C₆H₄)—C(O)—CH═CH₂, —(C₆H₄)—CH═O, —(C₆H₄)—S(O)₂—CH═CH₂, —(C₆H₄)—F, —(C₆H₄)—S(O)₂F, —O—(CH₂)₂—(C₆H₅); —C(O)—O—(C₆F₅), —CH₂—C(O)—O—(C₆F₅), —CH═O, and allyl.

10. Compound according to Formula I or II according to claim 1, wherein X¹ and X² together with the N to which they are attached form a heterocycle which is selected from:
azetidin-1-yl, 3-fluoro-azetidin-1-yl, 3-oxo-azetidin-1-yl, 3-chloro-azetidin-1-yl, 3-hydroxy-azetidin-1-yl, 2-(4-fluoro-phenyl)-azetidin-1-yl, 2-(4-chloro-phenyl)-azetidin-1-yl, 1-oxa-5-azaspiro[3.3]heptyl, 3-(N-(2-(4-fluorosulfony-phenyl)-ethyl)-amino-carbonyl)-azetidin-1-yl, pyrrolidin-1-yl, 3-hydroxymethyl-pyrrolidin-1-yl, (S)-3-hydroxymethyl-pyrrolidin-1-yl, (R)-3-hydroxymethyl-pyrrolidin-1-yl, 3-hydroxyethyl-pyrrolidin-1-yl, 3,3-difluoro-pyrrolidin-1-yl, 3-methoxy-pyrrolidin-1-yl, 3-ethoxy-pyrrolidin-1-yl, 3-hydroxy-pyrrolidin-1-yl, (R)-2-hydroxymethyl-pyrrolidin-1-yl, (S)-2-hydroxymethyl-pyrrolidin-1-yl, 2-Isopropyl-pyrrolidin-1-yl, 2-isobutyl-pyrrolidin-1-yl, (2S)-2-(bromomethyl)pyrrolidin-1-yl, 2-phenyl-pyrrolidin-1-yl, 2-benzyl-pyrrolidin-1-yl, 2-methyl-3-phenyl-pyrrolidin-1-yl, 3-hydroxy-3-phenyl-pyrrolidin-1-yl, 2-((S)-diphenyl-hydroxy-methyl)-pyrrolidin-1-yl, 2-((R)-diphenyl-hydroxy-methyl)-pyrrolidin-1-yl, 2-(2-methoxy-benzyl)-pyrrolidin-1-yl, (S)-2-(2-methoxy-benzyl)-pyrrolidin-1-yl, (R)-2-(2-methoxy-benzyl)-pyrrolidin-1-yl, 2-(1-methyl-1-phenyl-ethyl)-pyrrolidin-1-yl, 2-(2-methyl-benzyl)-pyrrolidin-1-yl, 2-(3-methyl-benzyl)-pyrrolidin-1-yl 2-(2-chloro-benzyl)-pyrrolidin-1-yl, 2-(4-chloro-benzyl)-pyrrolidin-1-yl, 2-methyl-pyrrolidin-1-yl, 2,3-dimethyl-pyrrolidin-1-yl, 3-ethyl-3-hydroxy-pyrrolidin-1-yl, 3-hydroxy-3-methyl-pyrrolidin-1-yl, 3-hydroxy-5-methyl-pyrrolidin-1-yl, 3-hydroxy-3-trifluoromethyl-pyrrolidin-1-yl, 2-(3-chloro-benzyl)-pyrrolidin-1-yl, 3-trifluoromethyl-pyrrolidin-1-yl, 3-carbamoyl-pyrrolidin-1-yl, (S)-3-carbamoyl-pyrrolidin-1-yl, (R)-3-carbamoyl-pyrrolidin-1-yl, 2-methyl-octahydro-indol-1-yl, 2,3-dihydro-indol-1-yl, 2-(2-chloro-benzyl)-pyrrolidin-1-yl, 2-methyl-3-phenyl-pyrrolidin-1-yl, (2S,3R)-2-methyl-3-phenyl-pyrrolidin-1-yl, (2S,3S)-2-methyl-3-phenyl-pyrrolidin-1-yl, (2R,3S)-2-methyl-3-phenyl-pyrrolidin-1-yl, (2R,3R)-2-methyl-3-phenyl-pyrrolidin-1-yl, 1-pyrrolidin-2-yl-acetic acid butyl ester, 1-pyrrolidine-2-carboxylic acid 2-chloro-benzylamide, 1-pyrrolidin-2-yl-acetic acid, (S)-5-hydroxymethyl-2-oxo-pyrrolidin-1yl, 2-Cyclopentyl-pyrrolidin-1-yl, 3-phenyl-2-oxo-pyrrolidin-1-yl, 5-(4-chloro-phenyl)-2-oxo-pyrrolidin1-yl, 2-(N-phenylaminocarbonyl)-pyrrolidin1-yl, 2-thiophen-3-yl-pyrrolidin-1-yl, 5-benzyl-2-oxo-pyrrolidin-1-yl, 4-benzyl-2-oxo-pyrrolidin-1-yl, 2-(2-phenylethyl)pyrrolidin-1-yl, (2S)-2-(methoxymethyl)pyrrolidin-1-yl, (2R)-2-(methoxymethyl)pyrrolidin-1-yl, 2-(methylsulfanylmethyl)pyrrolidin-1-yl, 2-vinyl-pyrrolidin-1-yl, 2-(N-(2-(4-fluorosulfony-phenyl)-ethyl)-amino-carbonyl)-pyrrolidin-1-yl, 2,2-diallyl-pyrrolidin-1-yl, 2-(4-phenyl-phenyl)-pyrrolidin-1-yl, 3-(N-(3-acryloylamino-phenyl)-amino-)-3-hydroxy-pyrrolidin-1-yl, 3-(4-acryloyl-phenyl)-3-hydroxy-pyrrolidin-1-yl, 3-(3-acryloyl-phenyl)-3-hydroxy-pyrrolidin-1-yl, 3-hydroxy-3-(3-vinylsulfonylphenyl)pyrrolidin-1-yl, 3-(3-fluorosulfony-phenyl)-3-hydroxy-pyrrolidin-1-yl, 3-(4-fluorosulfony-phenyl)-3-hydroxy-pyrrolidin-1-yl, 2-(2,3,4,5,6-pentafluorophenyl)oxycarbonyl-pyrrolidin-1-yl, 2-(2,3,4,5,6-pentafluorophenyl)oxycarbonylmethyl-pyrrolidin-1-yl, morpholin-4-yl, piperidin-1-yl, 3-fluoro-piperidin-1-yl, 3,3-difluoro-piperidin-1-yl, 3-chloro-piperidin-1-yl, 3-hydroxy-piperidin-1-yl, 3-methoxy-piperidin-1-yl, 3-hydroxy-3-phenyl-piperidin-1-yl, (S)-3-hydroxy-3-phenyl-piperidin-1-yl, (R)-3-hydroxy-3-phenyl-piperidin-1-yl, 3-benzyl-3-hydroxy-piperidin-1-yl, (R)-5,5-difluoro-3-hydroxy-piperidin-1-yl, (S)-5,5-difluoro-3-hydroxy-piperidin-1-yl, 2-(4-methoxy-benzyl)-piperidin-1-yl, 2-(2-methoxy-benzyl)-piperidin-1-yl, (R)-2-(2-methoxy-benzyl)-piperidin-1-yl, (S)-2-(2-methoxy-benzyl)-piperidin-1-yl, 2-(2-chloro-benzyl)-piperidin-1-yl, 2-Benzofuran-2-yl-piperidin-1-yl, 3,4-dihydro-2H-quinolin-1-yl, 2-methyl-2,3-dihydro-indol-1-yl, 6-(N-(2-(4-acryloylamino-phenyl)-ethyl)-amino-carbonyl)-piperidin-1-yl, (R)-2-(4-methoxy-benzyl)-piperidin-1-yl, (S)-2-(4-methoxy-benzyl)-piperidin-1-yl, 2-(N-(2-(4-fluorosulfony-phenyl)-ethyl)-amino-carbonyl)-piperidin-1-yl, [4-(2-{[1-(2-Amino-6-methylamino-pyrimidin-4-yl)-piperidine-4-carbonyl]-amino}-ethyl)-phenyl]-carbamic acid tert-butyl ester, 8-oxa-3-azabicyclo[3.2.1]octan-3-yl, 6′-fluoro-4′-hydroxy-spiro[azetidine-3,2′-chromane]-1-yl, and 6-(trifluoromethyl)-2-azabicyclo[3.1.0]hexan-2-yl.

11. Compound according to Formula I or II according to claim 1, wherein X¹ and X² together with the N to which they are attached form a heterocycle according to Formula 1, with the proviso that the N atom depicted in Formula 1 is the only N atom comprised by Formula 1; or wherein X¹ and X² together with the N to which they are attached form a heterocycle according to Formula 2, with the proviso that the N atom depicted in Formula 2 is the only N atom comprised by Formula 2; or wherein X¹ and X² together with the N to which they are attached form a heterocycle according to Formula 4, with the proviso that the N atom depicted in Formula 4 is the only N atom comprised by Formula 3; or wherein X¹ and X² together with the N to which they are attached form a heterocycle according to Formula 4, with the proviso that the N atom depicted in Formula 5 is the only N atom comprised by Formula 4.

12. A compound selected from:
[1-(2-amino-6-cyclopropylamino-pyrimidin-4-yl)-pyrrolidin-3-yl]-methanol 6-(3,3-difluoro-pyrrolidin-1-yl)-N⁴-methyl-pyrimidine-2,4-diamine;
N⁴-cyclopropyl-6-(3,3-difluoro-pyrrolidin-1-yl)-pyrimidine-2,4-diamine,
1-(2-amino-6-cyclopropylamino-pyrimidin-4-yl)-pyrrolidine-3-carboxylic acid amide-formate,
1-(2-amino-6-cyclopropylamino-pyrimidin-4-yl)-pyrrolidin-3-ol,
1-(2-amino-6-methylamino-pyrimidin-4-yl)-pyrrolidin-3-ol-formate,
(R)-1-(2-amino-6-methylamino-pyrimidin-4-yl)-pyrrolidin-2-yl]-methanol-formate,
6-(3-methoxy-pyrrolidin-1-yl)-N⁴-methyl-pyrimidine-2,4-diamine-formate;
6-(3-methoxy-pyrrolidin-1-yl)-N⁴-methyl-pyrimidine-2,4-diamine
N⁴-cyclopropyl-6-(3-methoxy-pyrrolidin-1-yl)-pyrimidine-2,4-diamine-formate,
N⁴-cyclopropyl-6-(3-methoxy-pyrrolidin-1-yl)-pyrimidine-2,4-diamine
[(R)-1-(2-amino-6-cyclopropylamino-pyrimidin-4-yl)-pyrrolidin-2-yl]-methano 1-formate,
[(R)-1-(2-amino-6-cyclopropylamino-pyrimidin-4-yl)-pyrrolidin-2-yl]-methano 1,
[(S)-1-(2-amino-6-methylamino-pyrimidin-4-yl)-pyrrolidin-2-yl]-methanol
[(S)-1-(2-amino-6-cyclopropylamino-pyrimidin-4-yl)-pyrrolidin-2-yl]-methanol,
6-(3,3-difluoro-piperidin-1-yl)-N⁴-methyl-pyrimidine-2,4-diamine,
6-(3,3-difluoro-piperidin-1-yl)-N⁴-methyl-pyrimidine-2,4-diamine,
6-(3-methoxy-piperidin-1-yl)-N⁴-methyl-pyrimidine-2,4-diamine,
1-(2-amino-6-methylamino-pyrimidin-4-yl)-3-benzyl-piperidin-3-ol,
(R)-1-(2-amino-6-methylamino-pyrimidin-4-yl)-5,5-difluoro-piperidin-3-ol,
(S)-1-(2-amino-6-methylamino-pyrimidin-4-yl)-5,5-difluoro-piperidin-3-ol,
6-azetidin-1-yl-N⁴-methyl-pyrimidine-2,4-diamine,
6-(3,3-difluoro-azetidin-1-yl)-N⁴-methyl-pyrimidine-2,4-diamine,
1-(2-amino-6-methylamino-pyrimidin-4-yl)-azetidin-3-one,
N⁴-methyl-6-(2-oxa-6-aza-spiro[3.3]hept-6-yl)-pyrimidine-2,4-diamine,
6-[2-(4-fluoro-phenyl)-azetidin-1-yl]-N⁴-methyl-pyrimidine-2,4-diamine;
N⁴-methyl-6-(8-oxa-3-azabicyclo[3.2.1]octan-3-yl)pyrimidine-2,4-diamine,
1-[2-amino-6-(methylamino)pyrimidin-4-yl]-6′-fluoro-spiro[azetidine-3,2′-chromane]-4′-ol,
(R)-1-(2-Amino-6-cyclopropylamino-pyrimidin-4-yl)-pyrrolidin-3-ol,
N4-Cyclopropyl-6-(2-phenyl-pyrrolidin-1-yl)-pyrimidine-2,4-diamine,
N4-Cyclopropyl-6-(2-methyl-octahydro-indol-1-yl)-pyrimidine-2,4-diamine,
1-(2-Amino-6-cyclopropylamino-pyrimidin-4-yl)-3-phenyl-pyrrolidin-3-ol,
N4-Cyclopropyl-6-(2-methyl-pyrrolidin-1-yl)-pyrimidine-2,4-diamine,
6-(2-Benzyl-pyrrolidin-1-yl)-N4-cyclopropyl-pyrimidine-2,4-diamine;
N4-Cyclopropyl-6-(2,3-dihydro-indol-1-yl)-pyrimidine-2,4-diamine,
6-[2-(2-Chloro-benzyl)-pyrrolidin-1-yl]-N4-cyclopropyl-pyrimidine-2,4-diamine,
(S)-1-(2-Amino-6-cyclopropylamino-pyrimidin-4-yl)-3-phenyl-pyrrolidin-3-ol,
(R)-1-(2-Amino-6-cyclopropylamino-pyrimidin-4-yl)-3-phenyl-pyrrolidin-3-ol,
N4-Cyclopropyl-6-(2,3-dimethyl-pyrrolidin-1-yl)-pyrimidine-2,4-diamine;
1-(2-Amino-6-cyclopropylamino-pyrimidin-4-yl)-3-ethyl-pyrrolidin-3-ol,
N4-Cyclopropyl-6-(2-methyl-3-phenyl-pyrrolidin-1-yl)-pyrimidine-2,4-diamine,
1-(2-Amino-6-cyclopropylamino-pyrimidin-4-yl)-3-trifluoromethyl-pyrrolidin-3-ol,
1-(2-Amino-6-cyclopropylamino-pyrimidin-4-yl)-3-methyl-pyrrolidin-3-ol,
(S)-1-(2-Amino-6-cyclopropylamino-pyrimidin-4-yl)-pyrrolidin-3-ol,
(3S,5R)-1-(2-Amino-6-cyclopropylamino-pyrimidin-4-yl)-5-methyl-pyrrolidin-3-ol,
(3R,5S)-1-(2-Amino-6-cyclopropylamino-pyrimidin-4-yl)-5-methyl-pyrrolidin-3-ol,
6-[(R)-2-(2-Chloro-benzyl)-pyrrolidin-1-yl]-N4-cyclopropyl-pyrimidine-2,4-diamine,
6-[(S)-2-(2-Chloro-benzyl)-pyrrolidin-1-yl]-N4-cyclopropyl-pyrimidine-2,4-diamine;
(S)-1-(2-Amino-6-cyclopropylamino-pyrimidin-4-yl)-3-trifluoromethyl-pyrrolidin-3-ol,
(R)-1-(2-Amino-6-cyclopropylamino-pyrimidin-4-yl)-3-trifluoromethyl-pyrrolidin-3-ol,
N4-Cyclopropyl-6-(3,4-dihydro-2H-quinolin-1-yl)-pyrimidine-2,4-diamine,
(R)-1-(2-Amino-6-cyclopropylamino-pyrimidin-4-yl)-3-methyl-pyrrolidin-3-ol,
(S)-1-(2-Amino-6-cyclopropylamino-pyrimidin-4-yl)-3-methyl-pyrrolidin-3-ol,
N4-Cyclopropyl-6-((2S,3R)-2-methyl-3-phenyl-pyrrolidin-1-yl)-pyrimidine-2,4-diamine,
N4-Cyclopropyl-6-((2S,3S)-2-methyl-3-phenyl-pyrrolidin-1-yl)-pyrimidine-2,4-diamine,
N4-Cyclopropyl-6-((2R,3S)-2-methyl-3-phenyl-pyrrolidin-1-yl)-pyrimidine-2,4-diamine,
N4-Cyclopropyl-6-((2R,3R)-2-methyl-3-phenyl-pyrrolidin-1-yl)-pyrimidine-2,4-diamine,
N4-Cyclopropyl-6-(2-methyl-2,3-dihydro-indol-1-yl)-pyrimidine-2,4-diamine,
(R)-1-(2-Amino-6-cyclopropylamino-pyrimidin-4-yl)-pyrrolidine-3-carboxylic acid amide,
(R)-1-(2-Amino-6-methylamino-pyrimidin-4-yl)-pyrrolidin-3-ol,
(S)-1-(2-Amino-6-cyclopropylamino-pyrimidin-4-yl)-pyrrolidine-3-carboxylic acid amide,
(S)-1-(2-Amino-6-methylamino-pyrimidin-4-yl)-pyrrolidin-3-ol,
4-(3,3-Difluoro-pyrrolidin-1-yl)-5,6,7,8-tetrahydro-pyrido[2,3-d]pyrimidin-2-ylamine,
[(R)-1-(2-Amino-6-methylamino-pyrimidin-4-yl)-pyrrolidin-3-yl]-methanol,
[(S)-1-(2-Amino-6-methylamino-pyrimidin-4-yl)-pyrrolidin-3-yl]-methanol,
[(R)-1-(2-Amino-6-cyclopropylamino-pyrimidin-4-yl)-pyrrolidin-3-yl]-methanol,
[(S)-1-(2-Amino-6-cyclopropylamino-pyrimidin-4-yl)-pyrrolidin-3-yl]-methanol,
6-(3,3-Difluoro-pyrrolidin-1-yl)-N4-ethyl-pyrimidine-2,4-diamine,
6-(3,3-Difluoro-pyrrolidin-1-yl)-N4-isopropyl-pyrimidine-2,4-diamine,
(R)-1-(2-Amino-5,6,7,8-tetrahydro-pyrido[2,3-d]pyrimidin-4-yl)-pyrrolidin-3-ol,
N4-Cyclopropylmethyl-6-(3,3-difluoro-pyrrolidin-1-yl)-pyrimidine-2,4-diamine,
6-(3,3-Difluoro-pyrrolidin-1-yl)-N4-propyl-pyrimidine-2,4-diamine,
(S)-1-(2-Amino-5,6,7,8-tetrahydro-pyrido[2,3-d]pyrimidin-4-yl)-pyrrolidin-3-ol-formate,
(S)-1-(2-Amino-5,6,7,8-tetrahydro-pyrido[2,3-d]pyrimidin-4-yl)-pyrrolidin-3-ol,
4-(3,3-Difluoro-pyrrolidin-I-yl)-5,6,7,8-tetrahydro-pyrido[2,3-d]pyrimidine,
(R)-1-(2-Amino-5,6,7,8-tetrahydro-pyrido[2,3-d]pyrimidin-4-yl)-pyrrolidine-3-carboxylic acid amide-trifluoroacetate,
(R)-1-(2-Amino-5,6,7,8-tetrahydro-pyrido[2,3-d]pyrimidin-4-yl)-pyrrolidine-3-carboxylic acid amide,
N4-Cyclopentylmethyl-6-(3,3-difluoro-pyrrolidin-1-yl)-pyrimidine-2,4-diamine,
6-(3,3-Difluoro-pyrrolidin-1-yl)-N4-[2-(2-methoxy-phenyl)-ethyl]-pyrimidine-2,4-diamine,
(S)-1-(2-Amino-5,6,7,8-tetrahydro-pyrido[2,3-d]pyrimidin-4-yl)-pyrrolidine-3-carboxylic acid amide-formate,
(S)-1-(2-Amino-5,6,7,8-tetrahydro-pyrido[2,3-d]pyrimidin-4-yl)-pyrrolidine-3-carboxylic acid amide,
1-(2-Amino-6-methylamino-pyrimidin-4-yl)-3-phenyl-piperidin-3-ol,
1-(2-Amino-6-cyclopropylamino-pyrimidin-4-yl)-pyrrolidine-2-carboxylic acid,
6-(2-Benzofuran-2-yl-piperidin-1-yl)-N4-methyl-pyrimidine-2,4-diamine,
6-[2-(2-Methoxy-benzyl)-pyrrolidin-1-yl]-N4-methyl-pyrimidine-2,4-diamine,
N4-Methyl-6-[2-(1-methyl-1-phenyl-ethyl)-pyrrolidin-I-yl]-pyrimidine-2,4-diamine,
6-[2-(4-Chloro-benzyl)-pyrrolidin-1-yl]-N4-methyl-pyrimidine-2,4-diamine,
6-[2-(3-Chloro-benzyl)-pyrrolidin-1-yl]-N4-methyl-pyrimidine-2,4-diamine,
(R)-1-(2-Amino-6-cyclopropylamino-pyrimidin-4-yl)-4-hydroxy-pyrrolidin-2-one,
[1-(2-Amino-6-cyclopropylamino-pyrimidin-4-yl)-pyrrolidin-2-yl]-acetic acid butyl ester,
1-(2-Amino-6-cyclopropylamino-pyrimidin-4-yl)-pyrrolidine-2-carboxylic acid 2-chloro-benzylamide,
N4-Methyl-6-[2-(2-methyl-benzyl)-pyrrolidin-1-yl]-pyrimidine-2,4-diamine,
(R)-1-(2-Amino-6-methylamino-pyrimidin-4-yl)-3-phenyl-piperidin-3-ol,
(S)-1-(2-Amino-6-methylamino-pyrimidin-4-yl)-3-phenyl-piperidin-3-ol,
6-[(S)-2-(2-Methoxy-benzyl)-pyrrolidin-1-yl]-N4-methyl-pyrimidine-2,4-diamine,
6-[(R)-2-(2-Methoxy-benzyl)-pyrrolidin-1-yl]-N4-methyl-pyrimidine-2,4-diamine,
1-(2-Amino-6-cyclopropylamino-pyrimidin-4-yl)-pyrrolidin-2-one,
6-[2-(4-Methoxy-benzyl)-piperidin-1-yl]-N4-methyl-pyrimidine-2,4-diamine,
1-(2-Amino-6-methylamino-pyrimidin-4-yl)-4-phenyl-pyrrolidin-2-one,
[1-(2-Amino-6-cyclopropylamino-pyrimidin-4-yl)-pyrrolidin-2-yl]-acetic acid,
6-[2-(2-Chloro-benzyl)-pyrrolidin-1-yl]-N4-methyl-pyrimidine-2,4-diamine,
(S)-1-(2-Amino-6-methylamino-pyrimidin-4-yl)-5-hydroxymethyl-pyrrolidin-2-one,
6-[2-(2-Methoxy-benzyl)-piperidin-1-yl]-N4-methyl-pyrimidine-2,4-diamine,
6-(2-Isopropyl-pyrrolidin-1-yl)-N4-methyl-pyrimidine-2,4-diamine-formate,
6-(2-Isopropyl-pyrrolidin-1-yl)-N4-methyl-pyrimidine-2,4-diamine,
6-(2-Cyclopentyl-pyrrolidin-1-yl)-N4-methyl-pyrimidine-2,4-diamine,
1-(2-Amino-6-methylamino-pyrimidin-4-yl)-3-phenyl-pyrrolidin-2-one,
1-(2-Amino-6-cyclopropylamino-pyrimidin-4-yl)-5-(4-chloro-phenyl)-pyrrolidin-2-one,
6-[2-(2-Chloro-benzyl)-piperidin-1-yl]-N4-methyl-pyrimidine-2,4-diamine,
6-[(R)-2-(2-Methoxy-benzyl)-piperidin-1-yl]-N4-methyl-pyrimidine-2,4-diamine,
6-[(S)-2-(2-Methoxy-benzyl)-piperidin-1-yl]-N4-methyl-pyrimidine-2,4-diamine,
4-(2-{[1-(2-Amino-6-methylamino-pyrimidin-4-yl)-azetidine-3-carbonyl]-amino}-ethyl)-benzenesulfonyl fluoride,
1-(2-Amino-6-cyclopropylamino-pyrimidin-4-yl)-pyrrolidine-2-carboxylic acid phenylamide,
[4-(2-{[1-(2-Amino-6-methylamino-pyrimidin-4-yl)-piperidine-4-carbonyl]-amino}-ethyl)-phenyl]-carbamic acid tert-butyl ester,
N4-Methyl-6-(2-thiophen-3-yl-pyrrolidin-1-yl)-pyrimidine-2,4-diamine,
N4-Methyl-6-[2-(3-methyl-benzyl)-pyrrolidin-1-yl]-pyrimidine-2,4-diamine,
1-(2-Amino-6-methylamino-pyrimidin-4-yl)-piperidine-4-carboxylic acid [2-(4-acryloylamino-phenyl)-ethyl]-amide,
1-(2-Amino-6-methylamino-pyrimidin-4-yl)-5-benzyl-pyrrolidin-2-one,
1-(2-Amino-6-methylamino-pyrimidin-4-yl)-4-benzyl-pyrrolidin-2-one,
6-[(R)-2-(4-Methoxy-benzyl)-piperidin-1-yl]-N4-methyl-pyrimidine-2,4-diamine,
6-[(S)-2-(4-Methoxy-benzyl)-piperidin-1-yl]-N4-methyl-pyrimidine-2,4-diamine,
1-(2-Amino-6-methylamino-pyrimidin-4-yl)-azetidine-3-carboxylic acid [2-(4-acryloylamino-phenyl)-ethyl]-amide,
4-(2-{[1-(2-Amino-6-cyclopropylamino-pyrimidin-4-yl)-pyrrolidine-2-carbonyl]-amino}-ethyl)-benzenesulfonyl fluoride,
4-(2-{[1-(2-Amino-6-methylamino-pyrimidin-4-yl)-piperidine-4-carbonyl]-amino}-ethyl)-benzenesulfonyl fluoride,
N4-methyl-6-[2-(2-phenylethyl)pyrrolidin-1-yl]pyrimidine-2,4-diamine;
6-[(2S)-2-(methoxymethyl)pyrrolidin-1-yl]-N4-methyl-pyrimidine-2,4-diamine,
N4-methyl-6-[2-(methylsulfanylmethyl)pyrrolidin-1-yl]pyrimidine-2,4-diamine,
6-[(2R)-2-(methoxymethyl)pyrrolidin-1-yl]-N4-methyl-pyrimidine-2,4-diamine,
[(2S)-1-[2-amino-6-(methylamino)pyrimidin-4-yl]pyrrolidin-2-yl]-diphenyl-methanol,
[(2R)-1-[2-amino-6-(methylamino)pyrimidin-4-yl]pyrrolidin-2-yl]-diphenyl-methanol,
6-(2-isobutylpyrrolidin-1-yl)-N4-methyl-pyrimidine-2,4-diamine,
6-[(2S)-2-(bromomethyl)pyrrolidin-1-yl]-N4-methyl-pyrimidine-2,4-diamine,
1-[2-amino-6-(methylamino)pyrimidin-4-yl]pyrrolidine-2-carbaldehyde,
6-(2,2-diallylpyrrolidin-1-yl)-N4-methyl-pyrimidine-2,4-diamine,
N4-methyl-6-[2-(4-phenylphenyl)pyrrolidin-1-yl]pyrimidine-2,4-diamine,
N-[3-[1-[2-amino-6-(methylamino)pyrimidin-4-yl]-3-hydroxy-pyrrolidin-3-yl]phenyl]prop-2-enamide,
1-[4-[1-[2-amino-6-(methylamino)pyrimidin-4-yl]-3-hydroxy-pyrrolidin-3-yl]phenyl]prop-2-en-1-one,
3-[1-[2-amino-6-(methylamino)pyrimidin-4-yl]-3-hydroxy-pyrrolidin-3-yl]benzaldehyde,
1-[2-amino-6-(methylamino)pyrimidin-4-yl]-3-(3-vinylsulfonylphenyl)pyrrolidin-3-ol,
3-[1-[2-amino-6-(methylamino)pyrimidin-4-yl]-3-hydroxy-pyrrolidin-3-yl]benzenesulfonyl fluoride,
4-[1-[2-amino-6-(methylamino)pyrimidin-4-yl]-3-hydroxy-pyrrolidin-3-yl]benzenesulfonyl fluoride,
1-[3-[1-[2-amino-6-(methylamino)pyrimidin-4-yl]-3-hydroxy-pyrrolidin-3-yl]phenyl]ethanone,
6-[2-(4-fluorophenyl)azetidin-1-yl]-N4-methyl-pyrimidine-2,4-diamine,
6-(3-benzyloxyazetidin-1-yl)-N4-methyl-pyrimidine-2,4-diamine,
(2,3,4,5,6-pentafluorophenyl) 1-[2-amino-6-(methylamino)pyrimidin-4-yl]pyrrolidine-2-carboxylate,
(2,3,4,5,6-pentafluorophenyl) 2-[1-[2-amino-6-(methylamino)pyrimidin-4-yl]pyrrolidin-2-yl]acetate,
N4-methyl-6-[6-(trifluoromethyl)-2-azabicyclo[3.1.0]hexan-2-yl]pyrimidine-2,4-diamine,
4-(3,3-Difluoro-pyrrolidin-1-yl)-5,6,7,8-tetrahydro-pyrido[2,3-d]pyrimidin-2-ylamine,
or pharmaceutically acceptable salt, solvate, tautomer, or stereoisomer thereof, preferably for use as a medicament, more preferably for use in the treatment of cancer.

13. A compound selected from
1-(2-amino-6-methylamino-pyrimidin-4-yl)-piperidin-3-ol, N⁴-methyl-6-(8-oxa-3-azabicyclo[3.2.1]octan-3-yl)pyrimidine-2,4-diamine,
N⁴-methyl-6-piperidin-1-yl-pyrimidine-2,4-diamine,
N⁴-methyl-6-pyrrolidin-1-yl-pyrimidine-2,4-diamine,
N⁴-methyl-6-morpholin-4-yl-pyrimidine-2,4-diamine,
1-(2-amino-6-methylamino-pyrimidin-4-yl)-pyrrolidin-3-ol,
[1-(2-amino-6-methylamino-pyrimidin-4-yl)-pyrrolidin-3-yl]-methanol, or pharmaceutically acceptable salt, stereoisomer, solvate thereof.

14. A compound according to Formula I or II as defined in claim 1 within a preparation.

15. A compound according to Formula I or II as defined in claim 1 suitable for use in the preparation of a medicament for use in the treatment of cancer, selected from lung cancer, breast cancer, colon cancer, pancreatic cancer, prostate cancer, ovarian cancer and bladder cancer, preferably lung cancer.

16. An inhibitor of the MTH1 protein, selected from a compound according to claim 1.

17. A pharmaceutical formulation comprising a therapeutically effective amount of a compound according to Formula I for use in the treatment of cancer as defined in claim 1.

18. A method of inhibiting MTH1 activity, comprising exposing the MTH1 protein to an effective amount of at least one of the compounds according to Formula I or II as defined in claim 1.

19. A method for preparing a medicament for treating cancer, comprising:
i. Determining a concentration at which a compound according to Formula I or II as defined in claim 1 effects 50% inhibition of MTH1 activity to be 100 nM or less, preferably 10 nM or less, more preferably 1 nM or less, and
ii. preparing a pharmaceutical composition comprising the compound for use in the treatment of cancer.
